(19) United States
(12) Patent Application Publication
(10) Pub. No.: US 2024/0269283 A1
(43) Pub. Date: Aug. 15, 2024

(54) ANTIGEN-BINDING POLYPEPTIDE TARGETING B7H3 AND APPLICATION THEREOF

(71) Applicant: NINGBO T-MAXIMUM BIOPHARMACEUTICALS CO., LTD., Ningbo (CN)

(72) Inventors: Xiaoyun SHANG, Ningbo (CN); Haijuan JIANG, Ningbo (CN); Dan WANG, Ningbo (CN); Jialu LI, Ningbo (CN); Shaowen MA, Ningbo (CN); Hui SHEN, Ningbo (CN); Li MA, Ningbo (CN); Weijie CHEN, Ningbo (CN)

(73) Assignee: NINGBO T-MAXIMUM BIOPHARMACEUTICALS CO., LTD., Ningbo (CN)

(21) Appl. No.: 18/575,650

(22) PCT Filed: Jun. 30, 2022
(86) PCT No.: PCT/CN2022/103070

§ 371 Date: Dec. 29, 2023

(30) Foreign Application Priority Data
Jul. 1, 2021 (CN) 202110749481.6

(51) Int. Cl. A61K 39/00; C07K 14/705; C07K 14/725; C07K 16/28; C12N 5/0783

(52) U.S. Cl. CPC A61K 39/4643; A61K 39/4611; A61K 39/4631; C07K 14/7051; C07K 14/70517; C07K 14/70578; C07K 16/2827; C12N 5/0636; C07K 2317/53; C07K 2319/02; C07K 2319/03; C12N 2510/00

(57) ABSTRACT

The present application relates to an antigen-binding polypeptide that specifically binds to B7H3, comprising at least one complementarity-determining region (CDR) of an antibody heavy chain variable region (VH), wherein the VH comprises an amino acid sequence set forth in SEQ ID NO: 25. The present application further relates to a chimeric antigen receptor comprising the antigen-binding polypeptide and a universal CAR-T cell comprising the chimeric antigen receptor. The CAR-T cell recognizes a surface antigen of a tumor cell and knocks out TCR and HLA-A genes expressed by the cell at the same time, so that the immune rejection caused by an allogeneic CAR-T therapy is reduced, the survival time of the cell is prolonged, and the anti-tumor effect is improved.

### TECHNICAL FIELD

The present application relates to the field of biomedicine, and in particular to an antigen-binding polypeptide targeting B7H3 and use thereof.

### BACKGROUND

Glioblastomas account for 15% of all brain tumors, and may originate from common brain cells or develop from low-grade astrocytomas. Generally, the survival time after diagnosis is 12 to 15 months, and only 3% to 7% of patients survive more than five years. Without treatment, the survival time is generally 3 months. About 3 out of every 100 thousand people are newly diagnosed with glioblastoma every year, which is the most common cancer of brain origin and the second most common brain tumor after meningioma.

With the development of tumor immunity theory and the progress of technology, the cell immunotherapy for tumors attracts more and more attention. CAR-T cell technology is a cell-based therapeutic approach that has produced excellent results in tumor immunotherapy, especially in the treatment of hematologic tumors. Genetically engineered T cells used in CAR-T immunotherapy can specifically recognize and kill tumor cells expressing specific antigens without being restricted by MHC. The CAR-T immunotherapy has achieved good effects in the treatment of various B-cell malignant tumors, for example, CD19-targeted CAR-T cells for the treatment of acute lymphocytic leukemia (ALL), chronic lymphocytic leukemia (CLL), and non-Hodgkin's lymphoma (NHL). At the same time, clinical use of CAR-T cells for multiple myeloma and relapsed/refractory multiple myeloma is in progress and shows encouraging results.

B7H3, also referred to as CD276, belongs to the immunomodulatory protein B7 family and is a type I membrane protein with an extracellular domain sequence similar to those of other B7 family members. The B7H3 gene is located on human chromosome 15 and consists of ten exons, of which exons 4 to 7 encode extracellular IgV-IgC domains. mRNA of B7H3 is expressed in various normal tissues and some tumor cell lines, and is not detectable in peripheral blood mononuclear cells (PBMCs). However, the expression of B7H3 can be induced on dendritic cells and monocytes by inflammatory cytokines (IFNγ) and compositions of PMA and ionomycin. Although B7H3 mRNA is widely expressed in normal tissues, the expression level of B7H3 protein is extremely low or absent in normal tissues, indicating that the expression of B7H3 protein is subjected to strict post-transcriptional regulation. In contrast, the B7H3 protein is overexpressed in various malignant tumors and is associated with poor prognosis, relatively high tumor grade and tumor metastasis, drug resistance, and low overall survival.

The differential expression of B7H3 between tumors and healthy tissues makes it very suitable as a therapeutic target, since targeting this antigen results in very limited side effects. The results of preclinical studies have shown that the inhibition or reduction of B7H3 protein expression in tumor cells can reduce cell proliferation and glycolysis, and increase drug sensitivity of tumor cells.

A CAR-T cell therapy targeting B7H3 has been studied. One preclinical study demonstrated that B7H3 CAR-T cells exhibit significant antitumor activity in vivo and can enable the established solid sarcomas in various xenograft models (including osteosarcoma, medulloblastoma, and Ewing's sarcoma) to be regressed.

However, autologous T cells of patients have difficulty expanding in vitro or reduced functions, resulting in an insufficient amount or poor quality of the CAR-T cell products prepared. Universal CAR-T cells are T cells isolated from healthy donors, and the prepared CAR-T cells not only have high amplification efficiency and strong activity, but also have an improved infection positive rate. However, the universal CAR-T also faces the problems of graft versus host disease (GVHD) and immune rejection. The CRISPR/Cas9 system is the most commonly used gene editing method, and can be used for producing T cells with TCR deficiency and HLA class I molecule deficiency, and for reducing immune rejection caused by allogeneic cell therapy.

### SUMMARY

The present invention aims to prepare a universal CAR-T cell targeting B7H3, which recognizes a surface antigen of a tumor cell and knocks out TCR and HLA-A genes expressed by the cell at the same time, so that the immune rejection caused by an allogeneic CAR-T therapy is reduced, the survival time of the cell is prolonged, and the anti-tumor effect is improved.

In one aspect, the present application provides an antigen-binding polypeptide that binds to B7H3 and comprises at least one complementarity-determining region (CDR) of an antibody heavy chain variable region (VH), wherein the VH comprises an amino acid sequence set forth in SEQ ID NO: 25.

In certain embodiments, the VH comprises an amino acid sequence set forth in SEQ ID NO: 26, SEQ ID NO: 27, SEQ ID NO: 28, or SEQ ID NO: 29.

In certain embodiments, the antigen-binding polypeptide comprises a VH, wherein the VH comprises a heavy chain complementarity-determining region 1 (HCDR1), a heavy chain complementarity-determining region 2 (HCDR2), and a heavy chain complementarity-determining region 3 (HCDR3), and the HCDR3 comprises an amino acid sequence set forth in SEQ ID NO: 7.

In certain embodiments, the HCDR3 comprises an amino acid sequence set forth in SEQ ID NO: 8 or SEQ ID NO: 9.

In certain embodiments, the HCDR2 comprises an amino acid sequence set forth in SEQ ID NO: 4.

In certain embodiments, the HCDR2 comprises an amino acid sequence set forth in SEQ ID NO: 5 or SEQ ID NO: 6.

In certain embodiments, the HCDR1 comprises an amino acid sequence set forth in SEQ ID NO: 1.

In certain embodiments, the HCDR1 comprises an amino acid sequence set forth in SEQ ID NO: 2 or SEQ ID NO: 3.

In certain embodiments, the VH comprises: the HCDR1 comprising the amino acid sequence set forth in SEQ ID NO: 1, the HCDR2 comprising the amino acid sequence set forth in SEQ ID NO: 4, and the HCDR3 comprising the amino acid sequence set forth in SEQ ID NO: 7.

In certain embodiments, the VH comprises:
i) the HCDR1 comprising the amino acid sequence set forth in SEQ ID NO: 2, the HCDR2 comprising the amino acid sequence set forth in SEQ ID NO: 5, and the HCDR3 comprising the amino acid sequence set forth in SEQ ID NO: 8; or
ii) the HCDR1 comprising the amino acid sequence set forth in SEQ ID NO: 3, the HCDR2 comprising the amino acid sequence set forth in SEQ ID NO: 6, and the HCDR3 comprising the amino acid sequence set forth in SEQ ID NO: 9.

In certain embodiments, the VH comprises a heavy chain framework region 1 (HFR1), a heavy chain framework region 2 (HFR2), a heavy chain framework region 3 (HFR3), and a heavy chain framework region 4 (HFR4), and the HFR1 comprises an amino acid sequence set forth in SEQ ID NO: 10.

In certain embodiments, the HFRI comprises an amino acid sequence set forth in SEQ ID NO: 11, SEQ ID NO: 12, SEQ ID NO: 13, or SEQ ID NO: 14.

In certain embodiments, the HFR2 comprises an amino acid sequence set forth in SEQ ID NO: 15.

In certain embodiments, the HFR2 comprises an amino acid sequence set forth in SEQ ID NO: 16 or SEQ ID NO: 17.

In certain embodiments, the HFR3 comprises an amino acid sequence set forth in SEQ ID NO: 18.

In certain embodiments, the HFR3 comprises an amino acid sequence set forth in SEQ ID NO: 19, SEQ ID NO: 20, or SEQ ID NO: 21.

In certain embodiments, the HFR4 comprises an amino acid sequence set forth in SEQ ID NO: 22.

In certain embodiments, the HFR4 comprises an amino acid sequence set forth in SEQ ID NO: 23 or SEQ ID NO: 24.

In certain embodiments, the VH comprises HFR1, HFR2, HFR3, and HFR4, and the HFR1, HFR2, HFR3, and HFR4 are selected from:
i) the HFR1 comprising the amino acid sequence set forth in SEQ ID NO: 11, the HFR2 comprising the amino acid sequence set forth in SEQ ID NO: 16, the HFR3 comprising the amino acid sequence set forth in SEQ ID NO: 19, and the HFR4 comprising the amino acid sequence set forth in SEQ ID NO: 23;
ii) the HFR1 comprising the amino acid sequence set forth in SEQ ID NO: 12, the HFR2 comprising the amino acid sequence set forth in SEQ ID NO: 16, the HFR3 comprising the amino acid sequence set forth in SEQ ID NO: 20, and the HFR4 comprising the amino acid sequence set forth in SEQ ID NO: 24;
iii) the HFR1 comprising the amino acid sequence set forth in SEQ ID NO: 13, the HFR2 comprising the amino acid sequence set forth in SEQ ID NO: 17, the HFR3 comprising the amino acid sequence set forth in SEQ ID NO: 21, and the HFR4 comprising the amino acid sequence set forth in SEQ ID NO: 23; and
vi) the HFR1 comprising the amino acid sequence set forth in SEQ ID NO: 14, the HFR2 comprising the amino acid sequence set forth in SEQ ID NO: 17, the HFR3 comprising the amino acid sequence set forth in SEQ ID NO: 20, and the HFR4 comprising the amino acid sequence set forth in SEQ ID NO: 24.

In certain embodiments, the VH comprises an amino acid sequence set forth in SEQ ID NO: 25.

In certain embodiments, the VH comprises an amino acid sequence set forth in SEQ ID NO: 26, SEQ ID NO: 27, SEQ ID NO: 28, or SEQ ID NO: 29.

In certain embodiments, the antigen-binding polypeptide includes an antibody or an antigen-binding fragment thereof.

In certain embodiments, the antibody includes a monoclonal antibody, a polyclonal antibody, a dimer, a polymer, a multispecific antibody, an intact antibody, an antibody fragment, a human antibody, a humanized antibody, or a chimeric antibody.

In certain embodiments, the antigen-binding fragment includes a Fab fragment, an Fv fragment, F(ab′)₂, a single-chain Fv (scFv), or a single-domain antibody (VHH). In another aspect, the present application provides a chimeric antigen receptor (CAR) comprising a targeting moiety, wherein the targeting moiety comprises the aforementioned antigen-binding polypeptide.

In certain embodiments, the targeting moiety includes a VHH.

In certain embodiments, the chimeric antigen receptor comprises a transmembrane domain, wherein the transmembrane domain comprises a transmembrane domain derived from one or more proteins selected from the group consisting of: CD8A, CD8B, CD28, CD3ε (CD3e), 4-1BB, CD4, CD27, CD7, PD-1, TRAC, TRBC, CD3ζ, CTLA-4, LAG-3, CD5, ICOS, OX40, NKG2D, 2B4 (CD244), FcεRIγ, BTLA, CD30, GITR, HVEM, DAP10, CD2, NKG2C, LIGHT, DAP12, CD40L (CD154), TIM1, CD226, DR3, CD45, CD80, CD86, CD9, CD16, CD22, CD33, CD37, CD64, and SLAM.

In certain embodiments, the transmembrane domain comprises a transmembrane domain derived from CD8A.

In certain embodiments, the transmembrane domain comprises an amino acid sequence set forth in any one of SEQ ID NO: 42 to SEQ ID NO: 90.

In certain embodiments, the chimeric antigen receptor comprises an intracellular co-stimulatory signaling domain, wherein the intracellular co-stimulatory signaling domain comprises an intracellular co-stimulatory signaling domain derived from one or more proteins selected from the group consisting of: CD28, CD137, CD27, CD2, CD7, CD8A, CD8B, OX40, CD226, DR3, SLAM, CDS, ICAM-1, NKG2D, NKG2C, B7H3, 2B4, FcεRIγ, BTLA, GITR, HVEM, DAP10, DAP12, CD30, CD40, CD40L, TIM1, PD-1, LFA-1, LIGHT, JAML, CD244, CD100, ICOS, CD40, and MyD88.

In certain embodiments, the intracellular co-stimulatory signaling domain is derived from a co-stimulatory signaling domain of 4-1BB.

In certain embodiments, the intracellular co-stimulatory signaling domain comprises an amino acid sequence set forth in any one of SEQ ID NO: 91 to SEQ ID NO: 123.

In certain embodiments, the chimeric antigen receptor comprises an intracellular signaling domain, wherein the intracellular signaling domain comprises an intracellular signaling domain derived from one or more proteins selected from the group consisting of: CD3ζ, CD3δ, CD3γ, CD3ε, CD79a, CD79b, FceRIγ, FceRIβ, FcγRIIa, bovine leukemia virus gp30, Epstein-Barr virus (EBV) LMP2A, simian immunodeficiency virus PBj14 Nef, DAP10, DAP-12, and a domain comprising at least one ITAM.

In certain embodiments, the intracellular signaling domain comprises a signaling domain derived from CD3ζ.

In certain embodiments, the intracellular signaling domain comprises an amino acid sequence set forth in any one of SEQ ID NO: 107, SEQ ID NO: 111, SEQ ID NO: 112, and SEQ ID NO: 124 to SEQ ID NO: 134.

In certain embodiments, the chimeric antigen receptor comprises a hinge region between the targeting moiety and the transmembrane domain, wherein the hinge region comprises a hinge region derived from one or more proteins selected from the group consisting of: CD28, IgG1, IgG4, IgD, 4-1BB, CD4, CD27, CD7, CD8A, PD-1, ICOS, OX40, NKG2D, NKG2C, FcεRIγ, BTLA, GITR, DAP10, TIM1, SLAM, CD30, and LIGHT.

In certain embodiments, the hinge region comprises a hinge region derived from CD8A.

In certain embodiments, the hinge region comprises an amino acid sequence set forth in any one of SEQ ID NO: 135 to SEQ ID NO: 156.

In certain embodiments, a non-targeting moiety of the chimeric antigen receptor comprises a transmembrane domain of CD8A molecule, a hinge region of CD8A, an intracellular co-stimulatory signaling domain of 4-1BB, and an intracellular signaling domain of CD3ζ.

In certain embodiments, the non-targeting moiety of the chimeric antigen receptor comprises an amino acid sequence set forth in SEQ ID NO: 30.

In certain embodiments, the chimeric antigen receptor further comprises a signal peptide fragment, wherein the C-terminus of the signal peptide fragment is linked to the N-terminus of the targeting moiety.

In certain embodiments, the signal peptide fragment includes a CD8A signal peptide fragment.

In certain embodiments, the signal peptide fragment comprises an amino acid sequence set forth in SEQ ID NO: 31.

In certain embodiments, the chimeric antigen receptor comprises an amino acid sequence set forth in any one of SEQ ID NO: 32, SEQ ID NO: 33, SEQ ID NO: 34, and SEQ ID NO: 35.

In another aspect, the present application provides one or more isolated nucleic acid molecules encoding the aforementioned antigen-binding polypeptide or the aforementioned chimeric antigen receptor.

In certain embodiments, the isolated nucleic acid molecule comprises a nucleotide sequence set forth in any one of SEQ ID NO: 36, SEQ ID NO: 37, SEQ ID NO: 38, and SEQ ID NO: 39.

In another aspect, the present application provides a vector comprising the aforementioned isolated nucleic acid molecule.

In certain embodiments, the vector is an expression vector.

In certain embodiments, the vector is selected from a DNA vector, an RNA vector, a plasmid, a lentiviral vector, an adenoviral vector, an adeno-associated viral vector, and a retroviral vector.

In another aspect, the present application provides a cell i) comprising the aforementioned isolated nucleic acid molecule or the aforementioned vector; and/or ii) expressing the aforementioned antigen-binding polypeptide or chimeric antigen receptor.

In another aspect, the present application provides an immune effector cell comprising the aforementioned nucleic acid molecule or the aforementioned vector, and/or expressing the aforementioned CAR.

In certain embodiments, the immune effector cell includes a human cell.

In certain embodiments, the immune effector cell includes a T cell, a B cell, a natural killer cell (NK cell), a macrophage, an NKT cell, a monocyte, a dendritic cell, a granulocyte, a lymphocyte, a leukocyte, and/or a peripheral blood mononuclear cell.

In certain embodiments, the immune effector cell includes an autologous or non-autologous immune effector cell.

In certain embodiments, the immune effector cell includes a modified immune effector cell.

In certain embodiments, the modified immune effector cell includes a cell that reduces immune rejection caused by allogeneic cell therapy.

In certain embodiments, the functions of a T cell antigen receptor (TCR) and major histocompatibility complexes (MHCI, MHCII) in the modified immune effector cell are inhibited in a T cell.

In certain embodiments, the modification comprises down-regulation of the expression and/or activity of one or more of immune rejection-related genes.

In certain embodiments, the immune rejection-related gene is selected from one or more of the following groups: TRAC, TRBC, HLA-A, HLA-B, B2M, and CIITA.

In certain embodiments, the expression and/or activity of the TRAC gene and the HLA-A gene in the modified immune effector cell is down-regulated as compared to a corresponding unmodified cell.

In certain embodiments, the expression and/or activity of the CIITA gene in the modified immune effector cell is not down-regulated as compared to the corresponding unmodified cell.

In certain embodiments, the expression and/or activity of the B2M gene in the modified immune effector cell is not down-regulated as compared to the corresponding unmodified cell.

In certain embodiments, the expression and/or activity of the TRAC gene and the HLA-A gene in the modified immune effector cell is down-regulated as compared to a corresponding wild-type cell.

In certain embodiments, the expression and/or activity of the B2M gene in the modified immune effector cell is not down-regulated as compared to the corresponding wild-type cell.

In certain embodiments, the expression and/or activity of the CIITA gene in the modified immune effector cell is not down-regulated as compared to the corresponding wild-type cell.

In certain embodiments, the down-regulation of the expression level and/or activity of the gene includes down-regulating the expression and/or activity of a nucleic acid molecule encoding the gene; and/or down-regulating the expression and/or activity of a protein product encoded by the gene.

In certain embodiments, the modification comprises: gene knockout, gene mutation, and/or gene silencing.

In certain embodiments, the modification comprises knocking out either of two TRAC alleles and knocking out either of two HLA-A alleles in the immune effector cell.

In certain embodiments, the modification comprises knocking out the two TRAC alleles and knocking out either of the two HLA-A alleles in the immune cell.

In certain embodiments, the modification comprises knocking out an exon of the TRAC gene and knocking out an exon of the HLA-A gene in the immune cell.

In certain embodiments, the modification comprises administering to the immune effector cell one or more substances selected from the group consisting of: antisense RNA, siRNA, shRNA, and a CRISPR/Cas9 system.

In certain embodiments, the modification comprises administering to the immune effector cell the CRISPR/Cas9 system.

In certain embodiments, the modification further comprises administering to the immune effector cell sgRNA targeting an exon portion of the TRAC gene.

In certain embodiments, the sgRNA targeting the exon portion of the TRAC gene comprises a nucleotide sequence set forth in any one of SEQ ID NO: 157 to SEQ ID NO: 171.

In certain embodiments, the modification comprises administering to the immune effector cell sgRNA targeting an exon portion of the HLA-A gene.

In certain embodiments, the sgRNA targeting the exon portion of the HLA-A gene comprises a nucleotide sequence set forth in any one of SEQ ID NO: 172 to SEQ ID NO: 212.

In certain embodiments, the modification further comprises administering to the cell a Cas enzyme.

In certain embodiments, the Cas enzyme includes a Cas9 protein.

In certain embodiments, the antisense RNA comprises a nucleotide sequence set forth in any one of SEQ ID NO: 213 to SEQ ID NO: 216.

In certain embodiments, the immune effector cell is an HLA-B homozygous cell. In certain embodiments, the HLA-B homozygote includes HLA-B*40 homozygote, HLA-B*15 homozygote, HLA-B*46 homozygote, HLA-B*13 homozygote, HLA-B*51 homozygote, HLA-B*58 homozygote, HLA-B*07 homozygote, HLA-B*35 homozygote, HLA-B*44 homozygote, HLA-B*52 homozygote, HLA-B*57 homozygote, HLA-B*54 homozygote, and HLA-B*55 homozygote.

In certain embodiments, the immune effector cell is an HLA-A homozygous or heterozygous cell.

In certain embodiments, the HLA-A homozygote or heterozygote includes HLA-A*02 homozygote, HLA-A*11 homozygote, HLA-A*02/A*11 heterozygote, or HLA-A*24 homozygote.

In another aspect, the present application provides a method for preparing an immune effector cell, which comprises introducing the aforementioned nucleic acid molecule or the aforementioned vector into the immune effector cell.

In certain embodiments, the method further comprises: modifying the immune effector cell before/after introducing the nucleic acid molecule according to any one of claims XX-XX or the vector according to any one of claims XX-XX into the immune effector cell, wherein the modification comprises down-regulation of the expression and/or activity of one or more of immune rejection-related genes.

In certain embodiments, the immune rejection-related gene is selected from one or more of the following groups: TRAC, TRBC, HLA-A, HLA-B, B2M, and CIITA.

In certain embodiments, the expression and/or activity of the TRAC gene and the HLA-A gene in the immune effector cell is down-regulated as compared to the expression and/or activity of a corresponding gene in a corresponding unmodified cell.

In certain embodiments, the expression and/or activity of the CIITA gene is not down-regulated as compared to the expression and/or activity of the corresponding gene in the corresponding unmodified cell.

In certain embodiments, the expression and/or activity of the B2M gene is not down-regulated as compared to the expression and/or activity of the corresponding gene in the corresponding unmodified cell.

In certain embodiments, the expression and/or activity of the TRAC gene and the HLA-A gene in the immune effector cell is down-regulated as compared to a corresponding wild-type cell.

In certain embodiments, the expression and/or activity of the CIITA gene is not down-regulated as compared to the corresponding wild-type cell.

In certain embodiments, the expression and/or activity of the B2M gene is not down-regulated as compared to the corresponding wild-type cell.

In certain embodiments, the down-regulation of the expression level and/or activity of the gene includes down-regulating the expression and/or activity of a nucleic acid molecule encoding the gene; and/or down-regulating the expression and/or activity of a protein product encoded by the gene.

In certain embodiments, the modification comprises: gene knockout, gene mutation, and/or gene silencing.

In certain embodiments, the modification comprises knocking out either of two TRAC alleles and knocking out either of two HLA-A alleles in the immune effector cell.

In certain embodiments, the modification comprises knocking out the two TRAC alleles and knocking out either of the two HLA-A alleles in the immune cell.

In certain embodiments, the modification comprises knocking out an exon of the TRAC gene and knocking out an exon of the HLA-A gene in the immune cell.

In certain embodiments, the modification comprises administering to the immune effector cell one or more substances selected from the group consisting of: antisense RNA, siRNA, shRNA, and a CRISPR/Cas9 system.

In certain embodiments, the modification comprises administering to the immune effector cell the CRISPR/Cas9 system.

In certain embodiments, the modification comprises administering to the immune effector cell sgRNA targeting an exon portion of the TRAC gene.

In certain embodiments, the sgRNA targeting the exon portion of the TRAC gene comprises a nucleotide sequence set forth in any one of SEQ ID NO: 157 to SEQ ID NO: 171.

In certain embodiments, the modification comprises administering to the immune effector cell sgRNA targeting an exon portion of the HLA-A gene.

In certain embodiments, the sgRNA targeting the exon portion of the HLA-A gene comprises a nucleotide sequence set forth in any one of SEQ ID NO: 172 to SEQ ID NO: 212.

In certain embodiments, the modification further comprises administering to the cell a Cas enzyme.

In certain embodiments, the Cas enzyme includes a Cas9 protein.

In certain embodiments, the antisense RNA comprises a nucleotide sequence set forth in any one of SEQ ID NO: 213 to SEQ ID NO: 216.

In certain embodiments, the immune effector cell includes a human cell.

In certain embodiments, the immune effector cell includes a T cell, a B cell, a natural killer cell (NK cell), a macrophage, an NKT cell, a monocyte, a dendritic cell, a granulocyte, a lymphocyte, a leukocyte, and/or a peripheral blood mononuclear cell.

In certain embodiments, the immune effector cell includes an autologous or non-autologous immune effector cell.

In certain embodiments, the cell is an HLA-B homozygous cell.

In certain embodiments, the HLA-B homozygote includes HLA-B*40 homozygote, HLA-B*15 homozygote, HLA-B*46 homozygote, HLA-B*13 homozygote, HLA-B*51 homozygote, HLA-B*58 homozygote, HLA-B*07 homozygote, HLA-B*35 homozygote, HLA-B*44 homozygote, HLA-B*52 homozygote, HLA-B*57 homozygote, HLA-B*54 homozygote, and HLA-B*55 homozygote.

In certain embodiments, the cell is an HLA-A homozygous or heterozygous cell.

In certain embodiments, the HLA-A homozygote or heterozygote includes HLA-A*02 homozygote, HLA-A*11 homozygote, HLA-A*02/A*11 heterozygote, or HLA-A*24 homozygote.

In another aspect, the present application provides use of the aforementioned chimeric antigen receptor, the aforementioned isolated nucleic acid molecule, the aforementioned vector, the aforementioned cell, or the aforementioned immune effector cell in the preparation of a CAR-T cell.

In another aspect, the present application provides a pharmaceutical composition comprising the aforementioned antigen-binding polypeptide, the aforementioned chimeric antigen receptor, the aforementioned isolated nucleic acid molecule, the aforementioned vector, the aforementioned cell, and/or the aforementioned immune effector cell, and optionally a pharmaceutically acceptable carrier.

In another aspect, the present application provides use of the aforementioned antigen-binding polypeptide, the aforementioned antigen chimeric receptor, the aforementioned isolated nucleic acid molecule, the aforementioned vector, the aforementioned cell, the aforementioned immune effector cell, and/or the aforementioned pharmaceutical composition in the treatment of a disease or disorder associated with the expression of B7H3.

In certain embodiments, the disease or disorder associated with the expression of B7H3 includes a disease or disorder associated with up-regulation of the expression of B7H3.

In certain embodiments, the disease or disorder associated with the expression of B7H3 includes cancer.

In certain embodiments, the cancer includes adrenocortical carcinoma, bladder cancer, breast cancer, cholangiocarcinoma, colorectal cancer, lymphoma, esophageal cancer, brain glioma, head and neck squamous cell carcinoma, kidney cancer, liver cancer, lung cancer, ovarian cancer, pancreatic cancer, prostate cancer, sarcoma, melanoma, gastric cancer, thymus cancer, or endometrial cancer.

In another aspect, the present application provides use of the aforementioned antigen-binding polypeptide, the aforementioned antigen chimeric receptor, the aforementioned isolated nucleic acid molecule, the aforementioned vector, the aforementioned cell, the aforementioned immune effector cell, and/or the aforementioned pharmaceutical composition in the preparation of a medicament for treating cancer.

In certain embodiments, the cancer includes a B7H3-positive cancer.

In certain embodiments, the cancer includes adrenocortical carcinoma, bladder cancer, breast cancer, cholangiocarcinoma, colorectal cancer, lymphoma, esophageal cancer, brain glioma, head and neck squamous cell carcinoma, kidney cancer, liver cancer, lung cancer, ovarian cancer, pancreatic cancer, prostate cancer, sarcoma, melanoma, gastric cancer, thymus cancer, or endometrial cancer.

In another aspect, the present application provides a method for preventing or treating a disease or disorder associated with the expression of B7H3, comprising administering to a subject in need thereof an effective amount of the aforementioned antigen-binding polypeptide, the aforementioned antigen chimeric receptor, the aforementioned isolated nucleic acid molecule, the aforementioned vector, the aforementioned cell, the aforementioned immune effector cell, and/or the aforementioned pharmaceutical composition.

In certain embodiments, the disease or disorder associated with the expression of B7H3 includes a disease or disorder associated with up-regulation of the expression of B7H3.

In certain embodiments, the disease or disorder associated with the expression of B7H3 includes cancer.

In certain embodiments, the cancer includes adrenocortical carcinoma, bladder cancer, breast cancer, cholangiocarcinoma, colorectal cancer, lymphoma, esophageal cancer, brain glioma, head and neck squamous cell carcinoma, kidney cancer, liver cancer, lung cancer, ovarian cancer, pancreatic cancer, prostate cancer, sarcoma, melanoma, gastric cancer, thymus cancer, or endometrial cancer.

Other aspects and advantages of the present application will be readily apparent to those skilled in the art from the following detailed description. Only exemplary embodiments of the present application have been shown and described in the following detailed description. As will be recognized by those skilled in the art, the content of the present application enables those skilled in the art to make changes to the specific embodiments disclosed without departing from the spirit and scope of the invention to which the present application pertains. Accordingly, descriptions in the drawings and specification are only illustrative rather than restrictive.

### BRIEF DESCRIPTION OF THE DRAWINGS

Specific features of the invention to which the present application pertains are set forth in appended claims. Features and advantages of the invention to which the present application pertains will be better understood by reference to the exemplary embodiments and drawings described in detail below. The drawings are briefly described as follows:

FIG. 1A shows an anti-B7H3 CAR gene lentiviral expression vector described in the present application;

FIG. 1B shows a strategy for constructing the anti-B7H3 UCAR-T cell of the present application;

FIG. 2 shows affinity curves of anti-B7H3 VHH antibodies described in the present application;

FIG. 3 shows ADCC function assay results of the anti-B7H3 VHH antibodies described in the present application;

FIGS. 4A-4C show cell phenotype assay results of anti-B7H3 UCAR-T cells described in the present application;

FIG. 5 shows a killing result of the anti-B7H3 UCAR-T cells described in the present application on target cells;

FIGS. 6A-6C show cytokine secretion assay results of the anti-B7H3 UCAR-T cells described in the present application co-cultured with the target cells;

FIG. 7 shows an in vivo anti-tumor effect of the anti-B7H3 UCAR-T cells described in the present application;

FIGS. 8A-8B show in vivo GVHD and rejection response results for targeting the anti-B7H3 UCAR-T cells described in the present application;

FIG. 9 shows off-target analysis of the anti-B7H3 UCAR-T cells described in the present application;

FIG. 10 shows chromosomal translocation analysis of the anti-B7H3 UCAR-T cells described in the present application;

FIG. 11 shows karyotype analysis of the anti-B7H3 UCAR-T cells described in the present application;

FIG. 12 shows residual Cas9 analysis of the anti-B7H3 UCAR-T cells described in the present application;

FIG. 13 shows Sanger sequencing results of TRAC gene after Sg9RNA editing in the present application;

FIG. 14 shows TA cloning assay results of the TRAC gene after Sg9RNA editing in the present application;

FIG. 15 shows flow cytometry assay results of the TRAC gene after Sg9RNA editing in the present application;

FIG. 16 shows the Sanger sequencing results of HLA-A02 gene after Sg2RNA editing in the present application;

FIG. 17 shows the Sanger sequencing results of HLA-A02 gene after Sg5RNA editing in the present application;

FIG. 18 shows the Sanger sequencing results of HLA-A11 gene after Sg10-3RNA editing in the present application;

FIG. 19 shows the Sanger sequencing results of HLA-A11 gene after Sg21RNA editing in the present application;

FIGS. 20A-20B show results of simultaneous knockout of HLA-A02 and TRAC in the modified immune effector cells of the present application;

FIGS. 21A-21B show protein levels of HLA-A02 and TRAC in the modified immune effector cells of the present application;

FIG. 22 shows mRNA levels of TRAC, HLA-A, B2M, and CIITA in the modified immune effector cells of the present application;

FIGS. 23A-23B show protein levels of B2M and CIITA in the modified immune effector cells of the present application;

FIGS. 24A-24D show protein levels of TRAC, HLA-A, B2M, and CIITA in the modified immune effector cells of the present application;

FIGS. 25A-25B show the knockout of TRAC and HLA-A at mRNA levels in the modified immune effector cells of the present application;

FIGS. 26A-26B show protein levels of CD69 and CD137 in the modified immune effector cells of the present application;

FIG. 27 shows the co-culture of the modified immune effector cells of the present application and NK cells;

FIG. 28 shows the expression level of IFN-γ in the modified immune effector cells of the present application;

FIGS. 29A-29D show protein levels of TRAC, HLA-A, B2M, and CIITA in the modified immune effector cells of the present application;

FIG. 30 shows the infection efficiency of the modified immune effector cells of the present application on CARs;

FIG. 31 shows amplification folds of the modified immune effector cells of the present application;

FIG. 32 shows a killing effect of the modified immune effector cells of the present application on CD19-positive target cells;

FIG. 33 shows a dosing regimen for administering the modified immune effector cells of the present application; and

FIG. 34 shows a killing effect of the modified immune effector cells of the present application on tumors in mice.

### DETAILED DESCRIPTION

The embodiments of the present invention are described below with reference to specific examples, and other advantages and effects of the present invention will be readily apparent to those skilled in the art from the disclosure of the present specification.

### Definitions of Terms

In the present application, the term “chimeric antigen receptor” or “CAR” generally refers to a group of polypeptides, generally two types in the simplest embodiment, which, when in an immune effector cell, provide the cell with specificity for a target cell (generally a cancer cell) and produce an intracellular signal. In some embodiments, the CAR comprises at least one extracellular antigen-binding domain (such as a VHH, scFv, or a portion thereof), a transmembrane domain, and a cytoplasmic signaling domain (also referred to herein as an “intracellular signaling domain”) that comprises a functional signaling domain derived from a stimulatory molecule and/or a co-stimulatory molecule as defined below. In some embodiments, the group of polypeptides are in the same polypeptide chain (e.g., comprise a chimeric fusion protein). In some embodiments, the group of polypeptides are not contiguous with each other, e.g., in different polypeptide chains. In some aspects, the group of polypeptides includes a dimerization switch that can couple the polypeptides to each other in the presence of a dimerization molecule, e.g., can couple an antigen-binding domain to an intracellular signaling domain. In one aspect, the stimulatory molecule of the CAR is a ζ chain associated with a T cell receptor complex. In one aspect, the cytoplasmic signaling domain comprises a primary signaling domain (e.g., a primary signaling domain of CD3-ζ). In one aspect, the cytoplasmic signaling domain further comprises one or more functional signaling domains derived from at least one co-stimulatory molecule as defined below. In one aspect, the co-stimulatory molecule may be selected from 4-1BB (i.e., CD137), CD27, ICOS, and/or CD28. In one aspect, the CAR comprises a chimeric fusion protein, which may comprise an extracellular antigen recognition domain, a transmembrane domain, and an intracellular signaling domain comprising a functional signaling domain derived from a stimulatory molecule. In one aspect, the CAR comprises a chimeric fusion protein, which may comprise an extracellular antigen recognition domain, a transmembrane domain, and an intracellular signaling domain comprising a functional signaling domain derived from a co-stimulatory molecule and a functional signaling domain derived from a stimulatory molecule. In one aspect, the CAR comprises a chimeric fusion protein, which may comprise an extracellular antigen recognition domain, a transmembrane domain, and an intracellular signaling domain comprising a functional signaling domain derived from one or more co-stimulatory molecules and a functional signaling domain derived from a stimulatory molecule. In one aspect, the CAR comprises a chimeric fusion protein, which may comprise an extracellular antigen recognition domain, a transmembrane domain, and an intracellular signaling domain comprising at least two functional signaling domains derived from one or more co-stimulatory molecules and a functional signaling domain derived from a stimulatory molecule. In one aspect, the CAR comprises an optional leader sequence at the amino-terminus (N-ter) of the CAR fusion protein. In one aspect, the CAR further comprises a leader sequence at the N-terminus of the extracellular antigen recognition domain, wherein the leader sequence is optionally cleaved from the antigen recognition domain (e.g., VHH) during cell processing and localizes the CAR to the cell membrane.

In the present application, the term “antibody” is generally meant to be used in the broadest sense and specifically encompasses monoclonal antibodies, polyclonal antibodies, dimers, polymers, multispecific antibodies (e.g., bispecific antibodies), and antibody fragments so long as they exhibit the desired biological activity (Miller et al., (2003) Jour. of Immunology 170: 4854-4861). The antibody may be a murine antibody, a human antibody, a humanized antibody, or a chimeric antibody, or derived from other species.

A full-length antibody typically refers to an antibody that consists of two “full-length antibody heavy chains” and two “full-length antibody light chains”. The “Full-length antibody heavy chain” generally refers to a polypeptide consisting of, from the N-terminus to the C-terminus, an antibody heavy chain variable domain (VH), an antibody constant heavy chain domain 1 (CH1), an antibody hinge region (HR), an antibody heavy chain constant domain 2 (CH2), and an antibody heavy chain constant domain 3 (CH3), abbreviated as VH-CH1-HR-CH2-CH3; and, in the case of antibodies of the IgE subclass, optionally further comprising an antibody heavy chain constant domain 4 (CH4). In some embodiments, the “full-length antibody heavy chain” is a polypeptide consisting of, from the N-terminus to the C-terminus, VH, CH1, HR, CH2, and CH3. The “full-length antibody light chain” is generally a polypeptide consisting of, from the N-terminus to the C-terminus, an antibody light chain variable domain (VL) and an antibody light chain constant domain (CL), abbreviated as VL-CL. The antibody light chain constant domain (CL) may be κ(kappa) or λ(lambda). The two full-length antibody chains are linked together by inter-polypeptide disulfide bonds between the CL domain and the CH1 domain and between the hinge regions of the full-length antibody heavy chains. Examples of typical full-length antibodies are natural antibodies such as IgG (e.g., IgG1 and IgG2), IgM, IgA, IgD, and IgE.

In the present application, the term “antigen-binding fragment” (also referred to herein as a “targeting moiety” or “antigen-binding moiety”) generally refers to a portion of an antibody molecule, which comprises amino acids responsible for specific binding between an antibody and an antigen. The portion of the antigen specifically recognized and bound by the antibody is referred to as an “epitope” described above. The antigen-binding domain may typically comprise an antibody light chain variable region (VL) and an antibody heavy chain variable region (VH); however, it does not necessarily comprise both. An Fd fragment, for example, has two VH regions and generally retains some of the antigen-binding functions of the intact antigen-binding domain. Examples of antigen-binding fragments of antibodies include: (1) a Fab fragment, a monovalent fragment having a VL, a VH, a constant light chain (CL) and a CH1 domain; (2) an F(ab′)₂ fragment, a bivalent fragment having two Fab fragments linked by a disulfide bridge at the hinge region; (3) an Fd fragment, having two VH and CH1 domains; (4) an Fv fragment, having VL and VH domains of a single arm of an antibody; (5) a dAb fragment (Ward et al., “Binding Activities of a Repertoire of Single Immunoglobulin Variable Domains Secreted From Escherichia coli”, Nature 341: 544-546 (1989), which is incorporated herein by reference in its entirety), having a VH domain; (6) an isolated complementarity-determining region (CDR); (7) a single-chain Fv (scFv), e.g., derived from an scFV-library. Although the two domains of the Fv fragment, VL and VH, are encoded by separate genes, they may be joined by a recombinant method using a synthetic linker that allows them to be prepared as a single protein chain in which the VL and VH regions pair to form monovalent molecules (referred to as single-chain Fv (scFv)) (see, e.g., Huston et al., “Protein Engineering of Antibody Binding Sites: Recovery of Specific Activity in an Anti-Digoxin Single-Chain Fv Analogue Produced in Escherichia coli”, Proc. Natl. Acad. Sci. USA 85: 5879-5883 (1988)); and (8) VHH, which relates to variable antigen-binding domains of heavy chain antibodies from Camelidae (camel, dromedary, llama, alpaca, etc.) (see Nguyen V. K. et al., 2000, The EMBO Journal, 19, 921-930; Muyldermans S., 2001, J Biotechnol., 74, 277-302 and a review of Vanl and schoot P. et al., 2011, Antiviral Research 92, 389-407). VHH may also be referred to as nanobody (Nb) and/or single-domain antibody. These antibody fragments are obtained using conventional techniques known to those skilled in the art, and assessed for the function in the same manner as for intact antibodies. An antigen-binding fragment targeting IL13Rα2 is also described in International Patent Application Publications WO2014072888A1 and WO2021041725A1, each of which is incorporated herein by reference in its entirety.

In the present application, the term “single-domain antibody” or “VHH” generally refers to a class of antibodies that lack an antibody light chain and have only a heavy chain variable region. In certain cases, the single-domain antibody may be derived from Bactrian camels, dromedaries, alpacas, llamas, nurse sharks, smooth dogfishes or rays (see, e.g., Kang Xiaozhen et al., Chinese Journal of Biotechnology, 2018, 34(12): 1974-1984). For example, the single-domain antibody may be derived from alpacas. The single-domain antibody may consist of a heavy chain variable region (VH). The term “heavy chain variable region” generally refers to the amino-terminal domain of the heavy chain of an antigen-binding fragment. The heavy chain variable region may be further divided into hypervariable regions termed complementarity-determining regions (CDRs), which are scattered over more conserved regions termed framework regions (FRs). Each heavy chain variable region may consist of three CDRs and four FRs arranged from the amino-terminus to the carboxyl-terminus in the following order: FR1, CDR1, FR2, CDR2, FR3, CDR3, and FR4. The heavy chain variable region comprises a binding domain that interacts with an antigen.

In the present application, the term “complementarity-determining region” (CDR) generally refers to a complementarity-determining region within a variable region of an antigen-binding fragment. In the present application, there are 3 CDRs present in the heavy chain variable region, and the CDRs are designated HCDR1, HCDR2 and HCDR3 for each variable region. The exact boundaries of those CDRs have been defined differently according to different systems. The system described by Kabat (Kabat et al., Sequences of Proteins of Immunological Interest, National Institutes of Health, Bethesda, Md. (1987) and (1991)) provides not only a clear residue numbering system applicable to any variable region of an antigen-binding fragment, but also precise residue boundaries defining 3 CDRs. Those CDRs may be referred to as Kabat CDRs. Chothia and colleagues (Chothia and Lesk, J. Mol. Biol., 196: 901-917 (1987) and Chothia et al., Nature 342: 877-883(1989)) found that although there is large diversity at the amino acid sequence level, certain sub-portions within Kabat CDRs take almost identical peptide backbone conformations. Those sub-portions were designated L1, L2 and L3 or H1, H2 and H3, wherein “L” and “H” refer to the light and heavy chain regions, respectively. Those regions may be referred to as Chothia CDRs, which have boundaries that overlap with Kabat CDRs. Other boundaries defining CDRs that overlap with Kabat CDRs have been described by Padlan (FASEB J. 9: 133-139 (1995)) and MacCallum (J Mol Biol 262 (5): 732-45 (1996)). In addition, other CDR boundary definitions may not strictly follow one of the above systems, but will nevertheless overlap with Kabat CDRs, although they may be shortened or lengthened according to predictions or experimental findings that a particular residue or a particular group of residues, or even the entire CDRs, do not significantly affect the antigen binding. In the present application, the IMGT numbering scheme is used.

In the present application, the term “FR” generally refers to the more highly conserved portions of antibody variable domains, which are referred to as framework regions. For example, the variable domains of natural heavy and light chains may each comprise four FR regions, namely four in VH (H-FR1, H-FR2, H-FR3, and H-FR4), and four in VL (L-FR1, L-FR2, L-FR3, and L-FR4). A “framework region” generally refers to a portion of the antibody variable region recognized in the art that is present between the more divergent (i.e., hypervariable) CDRs. Such framework regions are typically referred to as frameworks 1 to 4 (FR1, FR2, FR3, and FR4) and provide a backbone for presenting six CDRs (three from the heavy chain and three from the light chain) in the three-dimensional space to form an antigen-binding surface.

In the present application, the term “homology” may generally be equivalent to sequence “identity”. A homologous sequence may include an amino acid sequence that may be at least 80%, 85%, 90%, 99.1%, 99.2%, 99.3%, 99.4%, 99.5%, 99.6%, 99.7%, 99.8%, or 99.9% identical to a subject sequence. Generally, the homolog will comprise the same active site, etc. as the subject amino acid sequence. Homology may be considered in terms of similarity (i.e., amino acid residues having similar chemical properties/functions), or may be expressed in terms of sequence identity. In the present application, reference to a sequence having a percent identity of any one of the SEQ ID NOs of an amino acid sequence or a nucleotide sequence refers to a sequence having the percent identity over the entire length of the referenced SEQ ID NO. To determine sequence identity, sequence alignments can be performed by various means known to those skilled in the art, e.g., using BLAST, BLAST-2, ALIGN, NEEDLE, or Megalign (DNASTAR) software, etc. Those skilled in the art can determine appropriate parameters for alignment, including any algorithms required to achieve optimal alignment over the full length of the sequences being compared.

In the present application, the term “specific binding” when referring to the interaction of a binding molecule (e.g., an antibody) with its binding partner (e.g., an antigen) generally means that the interaction is dependent on the presence of a specific structure (e.g., an antigenic determinant or epitope) on the binding partner. In other words, the antibody will preferentially bind to or recognize a binding partner even when the binding partner is present in a mixture of other molecules or organisms. The binding may be mediated by covalent or non-covalent interactions or a combination of both. In other words, the term “specific binding” generally refers to immunospecific binding to an antigenic determinant or epitope and non-immunospecific binding to other antigenic determinants or epitopes. A binding molecule that immunospecifically binds to an antigen may bind to other peptides or polypeptides with relatively low affinity as determined by, for example, radioimmunoassay (RIA), enzyme-linked immunosorbent assay (ELISA), BIACORE, or other assays known in the art. The binding molecule or the fragment thereof that immunospecifically binds to an antigen may cross-react with a related antigen with the same epitope. In some cases, the binding molecule or the fragment thereof that immunospecifically binds to an antigen does not cross-react with other antigens.

In the present application, the term “KD” is used interchangeably with “KD” and generally refers to a dissociation equilibrium constant, in M (mol/L), of a particular antibody-antigen interaction. KD may be calculated from the concentration of substance AB and the concentration of substance A and substance B resulting from its dissociation: KD=c(A)×c(B)/c(AB). It can be seen from this equation that a larger KD indicates more dissociation and weaker affinity between substances A and B; conversely, a smaller KD indicates less dissociation and stronger affinity between substances A and B.

In the present application, the term “isolated nucleic acid molecule” generally refers to an isolated form of nucleotides, deoxyribonucleotides or ribonucleotides or analogs thereof of any length, isolated from their natural environment, or artificially synthesized.

In the present application, the term “vector” generally refers to a nucleic acid molecule capable of self-replicating in a suitable host, which transfers an inserted nucleic acid molecule into a host cell and/or between host cells. The vector may include vectors primarily for the insertion of DNA or RNA into a cell, vectors primarily for the replication of DNA or RNA, and vectors primarily for the expression of transcription and/or translation of DNA or RNA. The vector also includes vectors having a variety of the above-described functions. The vector may be a polynucleotide capable of being transcribed and translated into a polypeptide when introduced into a suitable host cell. Generally, the vector can produce the desired expression product by culturing an appropriate host cell containing the vector.

In the present application, the term “viral vector” is used broadly to refer to a nucleic acid molecule (e.g., transfer plasmid) or viral particle that mediates the transfer of nucleic acids. The nucleic acid molecule includes virus-derived nucleic acid elements that generally facilitate the transfer or integration of the nucleic acid molecules into the genome of a cell. The viral particle generally includes various viral components and sometimes further includes host cell components in addition to nucleic acids. The viral vector may refer to a virus or viral particle capable of transferring nucleic acids into a cell, or the transferred nucleic acid itself.

In the present application, the term “lentivirus” generally refers to a group (or genus) of complex retroviruses. Exemplary lentiviruses include, but are not limited to: human immunodeficiency virus (HIV; including HIV type 1 and HIV type 2); visna-maedivirus (VMV); caprine arthritis-encephalitis virus (CAEV); equine infectious anemia virus (EIAV); feline immunodeficiency virus (FIV); bovine immunodeficiency virus (BIV); and simian immunodeficiency virus (SIV). In one embodiment, an HIV-based vector backbone (i.e., HIV cis-acting sequence element) is preferred. In particular embodiments, the lentivirus is used to deliver a polynucleotide comprising the CAR to a cell.

In the present application, the term “host cell” or “cell” generally refers to an individual cell, cell line, or cell culture that may contain or has contained a vector comprising the isolated nucleic acid molecule described in the present application, or that is capable of expressing the isolated antigen-binding fragment described in the present application. The host cell may comprise progeny of a single host cell. Due to natural, accidental or deliberate mutations, progeny cells may not necessarily be identical in morphology or in genome to the original parent cell, but are capable of expressing the isolated antigen-binding fragment described herein. The host cell may be obtained by transfecting cells with the vector described herein in vitro. The host cell may be a prokaryotic cell (e.g., E. coli) or a eukaryotic cell (e.g., a yeast cell, a COS cell, a Chinese hamster ovary (CHO) cell, a HeLa cell, an HEK293 cell, a COS-1 cell, an NS0 cell, or a myeloma cell). For example, the host cell may be an E. coli cell. For example, the host cell may be a yeast cell. For example, the host cell may be a mammalian cell. For example, the mammalian cell may be a CHO-K1 cell.

In the present application, the term “T cell” or “T lymphocyte” may be any T cell, such as a cultured T cell, e.g., a primary T cell, or a T cell from the cultured T cell line, or a T cell obtained from a mammal (preferably a primate, species including monkey, dog, or human). If obtained from a mammal, the T cells may be obtained from a number of sources including, but not limited to, blood, bone marrow, lymph nodes, thymus, or other tissues or fluids. The T cell may also be enriched or purified. The T cell may be obtained by maturing a hematopoietic stem cell into a T cell in vitro or in vivo. In exemplary aspects, the T cell is a human T cell. In exemplary aspects, the T cell is a T cell isolated from a human. The T cell may be any type of T cell, including NKT cells, and may have any developmental stage, including but not limited to CD4+/CD8+ double positive T cells; CDA+ helper T cells; e.g., Th1 and Th2 cells, CD8⁺ T cells (e.g., cytotoxic T cells); peripheral blood mononuclear cells (PBMCs); peripheral blood leukocytes (PBLs); tumor infiltrating cells (TILs); memory T cells; untreated T cells, and the like. Preferably, the T cell is a CD8⁺ T cell or a CD4⁺ T cell. In some alternatives, the T cell is allogeneic (from different donors of the same species) to the recipient subject that receives the cell or cell to be received (e.g., the cells are in the form of a therapeutic composition); in some alternatives, the T cell is autologous (the donor and recipient are the same); in some alternatives, the T cell is syngeneic (the donor and recipient are different, but are homozygotic twins).

In the present application, the term “immune effector cell” generally refers to an immune cell involved in an immune response and performing an effector function. For example, the performing an effector function may include clearing foreign antigens, promoting an immune effector response, or the like. The immune effector cell may include plasma cells, T cells, B cells, natural killer (NK) cells, natural killer T (NKT) cells, mast cells, and myeloid-derived phagocytes.

The immune effector cell of the present application may be autologous/autogeneic (“self”) or non-autologous (“non-self”, e.g., allogeneic, syngeneic, or xenogeneic). In the present application, the term “autologous” generally refers to cells from the same subject. “Allogeneic” generally means that cells are of the same species as but genetically different from the cells to which they are compared. “Syngeneic” generally means that cells are from different subjects but genetically identical to the cells to which they are compared. “Xenogeneic” generally means that cells are of different species from the cell to which they are compared. In some embodiments, the cells of the present application are autologous or allogeneic.

In the present application, the term “modification” generally refers to changing the state or structure of the cell and/or a change in the state or structure of the cell. The change is generally compared to the state or structure of the corresponding unmodified cell. The change may include a change in endogenous gene expression level or function, for example, a down-regulation, up-regulation, or non-expression of the endogenous gene expression level of the cell by genetic engineering means, which may include homologous recombination, CRISPR/Cas9 system gene editing, or the like; the change may also include a change in cellular protein expression, structure, or function, for example, a change in the expression, structure, or function of the corresponding protein achieved by the change in the expression level or function of the endogenous gene, such as a change in the expression, structure or function of a protein achieved by the regulation of protein translation and post-translational modification; the change may also include the introduction of foreign genes, the expression of foreign proteins, and the like.

In the present application, the term “TRAC” generally refers to a T cell receptor a chain constant region (T cell receptor alpha constant). A T cell receptor (TCR) generally refers to a specific receptor located on the surface of the T cell, which is capable of recognizing antigens that bind to major histocompatibility complex (MHC) molecules. TCRs are generally composed of two different protein chains (i.e., heterodimers). In humans, TCRs in most T cells are composed of one a chain and one β chain (encoded by TRA and TRB, respectively), and this class of T cells is referred to as αβ T cells; and in a few T cells, TCRs are composed of γ chain and δ chain (encoded by TRG and TRD, respectively), and this class of T cells is referred to as γδ T cells. Generally, αβ T cells account for about 95% of the total T cells, γδ T cells account for about 5% of the total T cells, and the ratios vary during ontogenesis and in diseased states (e.g., leukemia), and also differ among species. Each chain constituting TCRs comprises a variable region and a constant region. In humans, the gene encoding α chain (TRA, e.g., information as shown by HGNC:12027) is located on chromosome 14 and consists of multiple gene fragments, including a variable fragment (V), a joining fragment (J), and a constant fragment (C). The TRAC gene generally refers to a gene sequence encoding the T cell receptor α chain constant region (C) (e.g., information as shown by HGNC:12029) and is located on chromosome 14 (14q11.2; 14:22,547,505-22,552,131). Generally, one of the genes of the variable fragments (V) encoding the N-fragment of the antigen recognition domain is rearranged with one of the joining fragments (J) to produce a functional V-region exon, which is transcribed and linked to the constant region (C) by splicing, thereby forming a coding sequence of the T cell receptor α chain.

In the present application, the term “major histocompatibility complex antigen” (“MHC”, also referred to as “human leukocyte antigen” (“HLA”) in humans) generally refers to a protein expressed on the surface of a cell that confers a unique antigenic identity to the cell. MHC/HLA antigens are target molecules that are recognized by T cells and NK cells as being derived from the same source of hematopoietic stem cells as immune effector cells (“self”) or as being derived from another source of hematopoietic repopulating cells (“non-self”). Two major classes of HLA antigens are recognized: HLA class I and HLA class II. HLA class I antigens (A, B, and C in humans) allow each cell to be recognized as “self”, while HLA class II antigens (DR, DP, and DQ in humans) are involved in reactions between lymphocytes and antigen-presenting cells. Both have been implicated in the rejection of transplanted organs. An important aspect of the HLA gene system is its polymorphism. Each gene for MHC class I (A, B, and C) and MHC class II (DP, DQ, and DR) exists in different alleles. HLA alleles are designated by numbers and subscripts. For example, two unrelated individuals may carry class I HLA-B genes B5 and Bw41, respectively. Allelic products differ in one or more amino acids of the α and/or β domains. A number of specific antibodies or nucleic acid reagents are used to type HLA haplotypes of individuals using leukocytes that express class I and class II molecules. Genes commonly used for HLA typing are six MHC class I and II proteins, i.e., two alleles for each of HLA-A, HLA-B, and HLA-DR. The HLA genes are clustered in a “super locus” present on chromosome position 6p21, wherein the “super locus” encodes 6 classical transplantation HLA genes and at least 132 protein-coding genes that play important roles in the regulation of the immune system as well as some other fundamental molecular and cellular processes. The complete locus measures roughly 3.6 Mb with at least 224 loci. One effect of such clustering is that a “haplotype”, i.e., a group of alleles present on a single chromosome, is inherited from one parent and tends to be inherited as a group. The group of alleles inherited from each parent form a haplotype, in which some alleles tend to be associated together. Identifying haplotypes of a patient may help predict the probability of finding a matching donor and help formulate a search strategy, because some alleles and haplotypes are more common than others and they are distributed at different frequencies in different racial and ethnic groups.

In the present application, “HLA-A” generally refers to a class of human leukocyte antigen polypeptide chains encoded by an HLA-A gene located on human chromosome 6p21.3 (e.g., information as shown by HGNC:4931). HLA-A is one of the three major polypeptide types that constitute MHC class I molecules on the surface of human cells, and others further include HLA-B and HLA-C. A heterodimer composed of an a chain encoded by the HLA-A gene and a β chain encoded by a B2M gene (β2-microglobulin) is an HLA-A class MHC I molecule. The α chain encoded by the HLA-A gene may comprise an α1 domain, an α2 domain, an α3 domain, a transmembrane region, and a cytoplasmic region, wherein the α1 domain and the α2 domain may bind to a peptide fragment so as to present the peptide fragment to an immune cell by the MHC I molecule (e.g., HLA-A class). In humans, similar to most mammals, the α chain of the MHC I molecule is polymorphic, and there are many variations in the primary structure thereof. As of December 2013, there are 2432 known HLA-A alleles in total, which encode 1740 active proteins and 117 inactive proteins. In the present application, HLA-A alleles may include sequence information on different HLA-A alleles recorded in the IMGT/HLA database version 3.38.0 (https://www.ebi.ac.uk/ipd/imgt/hla/) and designated by the WHO HLA Factor Nomenclature Committee.

In the present application, the term “HLA-B” generally refers to a part of the gene family of human leukocyte antigen (HLA) complexes. HLA is a human version of the major histocompatibility complex (MHC), and MHC is a gene family present in many species. The complex genes are divided into three basic groups: class I, class II, and class III. In humans, the HLA-B gene and the two related genes HLA-A and HLA-C are the major genes of MHC class I. The HLA-B gene is located in the cell band 21.3 of the short (p) arm of chromosome 6 from base pairs 31,353,871 to 31,357,211. HLA-B is one of the three major HLAs that should be matched between the donor and recipient. They are HLA-A, HLA-B (both are MHC class I), and HLA-DR (MHC class II). If two tissues have the same genes encoding the three HLAs, the possibility and severity of rejection are minimized. Hundreds of versions (alleles) of HLA-B are known, each version having a specific number (e.g., HLA-B27). Closely related alleles are grouped together, for example, at least 28 very similar alleles are subtypes of HLA-B27. These subtypes are designated as HLA-B*2701 to HLA-B*2728.

In the present application, the term “HLA-matched” refers to a donor-recipient pair in which none of the HLA antigens are mismatched between the donor and recipient, such as a donor providing a hematopoietic stem cell graft to a recipient in need of hematopoietic stem cell transplantation therapy. HLA-matched (i.e., in which all 6 alleles are matched) donor-recipient pairs have a reduced risk of graft rejection, because endogenous T cells and NK cells are less likely to recognize the incoming graft as foreign, and are thus less likely to generate an immune response against the graft.

In the present application, the term “HLA-mismatched” refers to a donor-recipient pair in which at least one HLA antigen (particularly with respect to HLA-A, HLA-B, and HLA-DR) is mismatched between the donor and recipient, such as a donor providing a hematopoietic stem cell graft to a recipient in need of hematopoietic stem cell transplantation therapy. In some embodiments, one haplotype is matched, and the other is mismatched. HLA-mismatched donor-recipient pairs may have an increased risk of graft rejection relative to HLA-matched donor-recipient pairs, because endogenous T cells and NK cells are more likely to recognize the incoming graft as foreign in the case of HLA-mismatched donor-recipient pairs, and such T cells and NK cells are thus more likely to generate an immune response against the graft.

In the present application, the term “B2M” generally refers to β2-microglobulin, which is one of the components of MHC class I molecules. β2 microglobulin (also referred to as β chain) may form an MHC class I molecule with an a chain encoded by HLA. B2M is generally expressed in all nucleated cells. In humans, β2 microglobulin is encoded by the B2M gene located at 15q21.1 (e.g., information as shown by HGNC:914).

In the present application, the term “CIITA” generally refers to a transactivator of a class II major histocompatibility complex (MHC II). The transactivator may be a protein having an acidic transcriptional activation domain, 4 LRRs (leucine-rich repeats), and a GTP binding domain. The protein may be located in the cell nucleus, act as a positive regulator of the gene transcription of the class II major histocompatibility complex (MHC II), and be referred to as a “master control factor” for the expression of these genes. The protein may also bind to GTP and utilize the binding to GTP to transport itself into the cell nucleus, where it generally functions by acetyltransferase (AT) activity in a coactivator-like manner. In humans, the protein is encoded by a gene located at 16p13.13 (e.g., information as shown by HGNC:7067), and several transcript variants encoding different isoforms can be produced.

In the present application, the term “wild-type cell” generally refers to a cell that naturally occurs or is of natural origin.

In the present application, the term “nucleic acid” or “polynucleotide” or “nucleic acid molecule” generally refers to deoxyribonucleic acid (DNA) or ribonucleic acid (RNA) and polymers thereof in either single-stranded or double-stranded form. Unless specifically limited, the term may include nucleic acids comprising analogs of natural nucleotides that have similar binding properties as the reference nucleic acid (e.g., with sequence information shown) and are metabolized in a manner similar to naturally occurring nucleotides. Unless otherwise indicated, the sequence of a nucleic acid may include conservatively modified variants thereof (e.g., degenerate codon substitutions), alleles, orthologs, SNPs, and complementary sequences, as well as the sequences explicitly indicated.

In the present application, the term “expression” generally refers to the transcription and/or translation of a particular nucleotide sequence.

In the present application, the term “gene mutation” generally refers to a change in the composition or arrangement order of base pairs occurring in the structure of genes, such as a point mutation caused by a single base change, or deletion, duplication, insertion, and the like of a plurality of bases.

In the present application, the term “gene silencing” generally refers to the prevention of the expression of certain genes by regulatory mechanisms. The gene silencing may primarily include two types: one is transcriptional gene silencing (TGS) caused by factors such as DNA methylation, heterochromatization, and position effect at the transcriptional level, and the other is post-transcriptional gene silencing (PTGS), which is the effect on gene expression at the post-transcriptional level by specific intervention on the target RNA. Generally, when a gene is silenced, the expression of the corresponding gene is down-regulated/reduced. When a gene is knocked out, it is generally not expressed. For example, the expression of a specific gene in a cell disappears when all alleles of the specific gene are knocked out. Gene silencing is generally considered to be a gene knockdown mechanism, and methods commonly used to silence genes may be, for example, RNAi and the like.

In the present application, the term “endogenous” refers to any substance derived from or produced within an organism, a cell, a tissue, or a system.

In the present application, the term “exogenous” refers to any substance introduced from or produced outside of an organism, a cell, a tissue, or a system.

In the present application, the term “antisense RNA” generally refers to a single-stranded RNA complementary to a transcript mRNA (messenger RNA). The antisense RNA may inhibit the expression of genes by binding to mRNA. For example, the binding of the antisense RNA to the target mRNA results in an increased sensitivity of the double-stranded RNA molecule to RNA enzyme III, and causes the degradation of the double-stranded RNA molecule. For example, the antisense RNA binds to an upstream non-coding region of mRNA, thereby directly inhibiting the translation of the target mRNA.

In the present application, the term “siRNA” generally refers to the abbreviation of small interfering RNA or short interfering RNA. siRNA is a class of double-stranded, non-coding RNA molecules that are about 18-28 base pairs in length and may cause the degradation of mRNA by the complementary binding to mRNA, thereby interfering with the expression of a specific gene. In certain embodiments, siRNA may be a product obtained by treating a long double-stranded RNA or shRNA with Dicer enzyme. In certain embodiments, siRNA enters a cell to form an RNA-induced silencing complex (RISC) with other proteins, the sense strand is degraded, and the antisense strand may bind to a complementary targeting sequence, thereby achieving gene silencing.

In the present application, the term “shRNA” generally refers to the abbreviation of short hairpin RNA, i.e., “short hairpin RNA”. shRNA generally comprises two short inverted repeats separated by a stem-loop sequence to form a hairpin structure. Generally, shRNA may further comprise 5-6 T bases as transcription terminators for RNA polymerase III. In certain embodiments, shRNA may enter a cell via a viral vector or plasmid, and be transcribed under the action of polymerase Il or polymerase III. The transcripts are exported from the cell nucleus (generally via Exportin 5), and then transported to RISC after Dicer treatment. The sense strand is degraded, and the antisense strand may bind to a complementary targeting sequence, thereby achieving gene silencing.

In the present application, the term “CRISPR/Cas system” generally refers to a group of molecules comprising an RNA-guided nuclease or other effector molecules and a gRNA molecule, and the molecules are capable of directing and realizing modification of a nucleic acid by the RNA-guided nuclease or other effector molecules at a target sequence, e.g., causing degradation of the target sequence. In certain embodiments, the CRISPR system comprises gRNA and a Cas protein, e.g., Cas9 protein. A system comprising Cas9 or a functional mutant thereof is referred to herein as “Cas9 system” or “CRISPR/Cas9 system”. In certain embodiments, the gRNA molecule and Cas molecule may be complexed to form a ribonucleoprotein (RNP) complex.

In the present application, the terms “gRNA molecule”, “guide RNA”, “instruction RNA”, “direct RNA”, “guide RNA molecule”, and “gRNA” can be used interchangeably and generally refer to a nucleic acid molecule capable of facilitating the specific guidance of the RNA-guided nuclease or other effector molecules (generally complexed with a gRNA molecule) onto the target sequence. In certain embodiments, the guidance is achieved by the hybridization of a portion of gRNA with DNA (e.g., via a gRNA guide domain) and by the binding of a portion of the gRNA molecule to the RNA-guided nuclease or other effector molecules (e.g., at least via gRNAtracr). In certain embodiments, the gRNA molecule consists of a single, contiguous polynucleotide molecule, referred to herein as a “single guide RNA”, “sgRNA”, or the like. In other embodiments, the gRNA molecule consists of multiple (e.g., two) polynucleotide molecules that are themselves capable of association (typically by hybridization), referred to herein as a “dual guide RNA”, “dgRNA”, or the like.

In the present application, the term “Cas protein” generally refers to an enzyme responsible for cleaving DNA in the CRISPR/Cas system. The enzyme may include enzymes from types I, II, and III CRISPR/Cas systems, e.g., Cas3, Cas9, and Cas10.

In the present application, the term “Cas9 protein” generally refers to an enzyme responsible for cleaving DNA, which is from the bacterial type II CRISPR/Cas system. Cas9 may include wild-type proteins and functional mutants thereof.

In the present application, “allele” generally refers to a form of a gene sequence at a locus that may have different variations. The locus is also referred to as a gene site or site and refers to a fixed position on a chromosome, e.g., where a gene is located. The arrangement of a locus in the genome is referred to as a genetic map. In the present application, the term “homozygote” generally refers to a genotype individual in which two alleles of homologous chromosomes are identical at the same locus. A pair of opposing genes may have individuals with two genotypes, AA and aa.

In the present application, the term “heterozygote” generally refers to a genotype individual in which two alleles at the same site on homologous chromosomes in a diploid are not identical, such as Aa. Heterozygous genotypes are generally more adaptive than homozygous dominant or homozygous recessive genotypes, and such phenomenon is referred to as heterozygote advantage.

In the present application, the terms “tumor” and “cancer” are used interchangeably and generally refer to a disease characterized by rapid and uncontrolled growth of abnormal cells. Cancer cells can spread to other parts of the body locally or through the bloodstream and lymphatic system. Examples of various cancers are described herein and include, but are not limited to, breast cancer, prostate cancer, ovarian cancer, cervical cancer, skin cancer, pancreatic cancer, colorectal cancer, kidney cancer, liver cancer, brain cancer, lymphoma, leukemia, lung cancer, and the like. The term “cancer” or “tumor” includes premalignant and malignant cancers and tumors, and also encompasses solid tumors and non-solid tumors.

In the present application, the term “pharmaceutically acceptable” generally refers to those compounds, materials, compositions, and/or dosage forms which are, commensurate with a reasonable benefit/risk ratio, and suitable, within the scope of sound medical judgment, for use in contact with the tissues of human beings and animals without excessive toxicity, irritation, allergic response, or other problems or complications.

In the present application, the term “pharmaceutically acceptable carrier” generally refers to any of those carriers conventionally used and is limited only by physical or chemical factors (such as solubility and lack of reactivity with active binding agents) and by the route of administration. The pharmaceutically acceptable carrier, such as a vehicle, an adjuvant, an excipient, and a diluent, described herein is well known to those skilled in the art and readily available to the public. In one aspect, the pharmaceutically acceptable carrier is one that is chemically inert to an active ingredient of a pharmaceutical composition and one that does not have adverse side effects or toxicity under the conditions of use. In some embodiments, the carrier does not produce an adverse, allergic, or other untoward reaction when administered to an animal or human. In some aspects, the pharmaceutical composition does not comprise pyrogens and other impurities that may be harmful to humans or animals. The pharmaceutically acceptable carrier includes any and all solvents, dispersion media, coatings, antibacterial and antifungal agents, isotonic and absorption delaying agents, and the like; the use of the pharmaceutically acceptable carrier is well known in the art.

The acceptable carriers, excipients, or stabilizers are non-toxic to recipients and are preferably inert at the doses and concentrations employed, and include buffers, such as phosphate, citrate, or other organic acids; antioxidants, such as ascorbic acid; low molecular weight polypeptides; proteins, such as serum albumin, gelatin, or immunoglobulin; hydrophilic polymers, such as polyvinylpyrrolidone; amino acids, such as glycine, glutamine, asparagine, arginine, or lysine; monosaccharides, disaccharides, and other carbohydrates including glucose, mannose, or dextrin; chelating agents, such as EDTA; sugar alcohols, such as mannitol or sorbitol; salt-forming counter-ions, such as sodium; and/or non-ionic surfactants, such as Tween, Pluronics, or polyethylene glycol (PEG).

In the present application, the term “effective amount” or “effective dose” generally refers to an amount sufficient to achieve, or at least partially achieve, a desired effect. “Therapeutically effective amount” or “therapeutically effective dose” of a drug or therapeutic agent is generally any amount of drug that promotes the regression of a disease (as evidenced by a decrease in the severity of symptoms of the disease, an increase in the frequency and duration of the asymptomatic phase of the disease, or the prevention of damage or disability due to the development of the disease) when used alone or in combination with another therapeutic agent.

“Therapeutically effective amount” or “effective amount” of an anti-B7H3 CAR-T cell is also an amount or dose that has a therapeutically beneficial effect over any toxic or deleterious effects, such as CRS, of the anti-B7H3 CAR-T cell. The term “therapeutically effective amount” includes an amount effective to “treat” a subject (e.g., a patient). In one embodiment, the therapeutically effective dose is the minimum effective dose (MED) of the anti-B7H3 CAR-T cell for treating multiple myeloma in the subject. In one embodiment, the therapeutically effective dose is the maximum tolerated dose (MTD) of the anti-B7H3 CAR-T cell that does not cause the subject to have unresolved CRS.

In the present application, the term “comprise” or “comprising” generally means including, summarizing, containing or encompassing. In certain cases, the term also means “being” or “consisting of . . . ”.

In the present application, the term “about” generally means varying by 0.5%-10% above or below the stated value, for example, varying by 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% above or below the stated value.

In the present application, the term “subject” generally refers to a human or non-human animal, including but not limited to cats, dogs, horses, pigs, cows, sheep, rabbits, mice, rats, monkeys, and the like.

### DETAILED DESCRIPTION OF THE INVENTION

### Antigen-Binding Polypeptide

In one aspect, the present application provides an antigen-binding polypeptide comprising at least one complementarity-determining region (CDR) of an antibody heavy chain variable region (VH), wherein the VH comprises an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 25.

In certain embodiments, the VH comprises an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 26, SEQ ID NO: 27, SEQ ID NO: 28, or SEQ ID NO: 29.

In certain embodiments, the antigen-binding polypeptide comprises a VH, wherein the VH comprises a heavy chain complementarity-determining region 1 (HCDR1), a heavy chain complementarity-determining region 2 (HCDR2), and a heavy chain complementarity-determining region 3 (HCDR3), and the HCDR3 comprises an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 7. For example, the HCDR3 may comprise an amino acid sequence set forth in SEQ ID NO: 7.

In certain embodiments, the HCDR3 comprises an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 8 or SEQ ID NO: 9. For example, the HCDR3 may comprise an amino acid sequence set forth in SEQ ID NO: 8 or SEQ ID NO: 9.

In certain embodiments, the HCDR2 comprises an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 4. For example, the HCDR2 may comprise an amino acid sequence set forth in SEQ ID NO: 4.

In certain embodiments, the HCDR2 comprises an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 5 or SEQ ID NO: 6. For example, the HCDR2 may comprise an amino acid sequence set forth in SEQ ID NO: 5 or SEQ ID NO: 6.

In certain embodiments, the HCDRI comprises an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 1. For example, the HCDR1 may comprise an amino acid sequence set forth in SEQ ID NO: 1.

In certain embodiments, the HCDR1 comprises an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 2 or SEQ ID NO: 3. For example, the HCDR1 may comprise an amino acid sequence set forth in SEQ ID NO: 2 or SEQ ID NO: 3.

In certain embodiments, the VH comprises: HCDR1 comprising an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 1, HCDR2 comprising an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 4, and HCDR3 comprising an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 7.

For example, the VH may comprise: the HCDR1 comprising the amino acid sequence set forth in SEQ ID NO: 1, the HCDR2 comprising the amino acid sequence set forth in SEQ ID NO: 4, and the HCDR3 comprising the amino acid sequence set forth in SEQ ID NO: 7.

In certain embodiments, the VH comprises:
i) the HCDR1 comprising the amino acid sequence set forth in SEQ ID NO: 2, the HCDR2 comprising the amino acid sequence set forth in SEQ ID NO: 5, and the HCDR3 comprising the amino acid sequence set forth in SEQ ID NO: 8; or
ii) the HCDR1 comprising the amino acid sequence set forth in SEQ ID NO: 3, the HCDR2 comprising the amino acid sequence set forth in SEQ ID NO: 6, and the HCDR3 comprising the amino acid sequence set forth in SEQ ID NO: 9.

In certain embodiments, the VH comprises a heavy chain framework region 1 (HFR1), a heavy chain framework region 2 (HFR2), a heavy chain framework region 3 (HFR3), and a heavy chain framework region 4 (HFR4), wherein the HFR1 comprises an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 10. For example, the HFRI may comprise an amino acid sequence set forth in SEQ ID NO: 10.

In certain embodiments, the HFR1 comprises an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 11, SEQ ID NO: 12, SEQ ID NO: 13, or SEQ ID NO: 14. For example, the HFRI comprises an amino acid sequence set forth in SEQ ID NO: 11, SEQ ID NO: 12, SEQ ID NO: 13, or SEQ ID NO: 14.

In certain embodiments, the HFR2 comprises an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 15. For example, the HFR2 may comprise an amino acid sequence set forth in SEQ ID NO: 15.

In certain embodiments, the HFR2 comprises an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 16 or SEQ ID NO: 17. For example, the HFR2 may comprise an amino acid sequence set forth in SEQ ID NO: 16 or SEQ ID NO: 17.

In certain embodiments, the HFR3 comprises an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 18. For example, the HFR3 may comprise an amino acid sequence set forth in SEQ ID NO: 18.

In certain embodiments, the HFR3 comprises an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 19, SEQ ID NO: 20, or SEQ ID NO: 21. For example, the HFR3 may comprise an amino acid sequence set forth in SEQ ID NO: 19, SEQ ID NO: 20, or SEQ ID NO: 21.

In certain embodiments, the HFR4 comprises an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 22. For example, the HFR4 may comprise an amino acid sequence set forth in SEQ ID NO: 22.

In certain embodiments, the HFR4 comprises an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 23 or SEQ ID NO: 24. For example, the HFR4 may comprise an amino acid sequence set forth in SEQ ID NO: 23 or SEQ ID NO: 24.

In certain embodiments, the VH comprises HFR1, HFR2, HFR3, and HFR4, and the HFR1, HFR2, HFR3, and HFR4 are selected from:
i) the HFR1 comprising the amino acid sequence set forth in SEQ ID NO: 11, the HFR2 comprising the amino acid sequence set forth in SEQ ID NO: 16, the HFR3 comprising the amino acid sequence set forth in SEQ ID NO: 19, and the HFR4 comprising the amino acid sequence set forth in SEQ ID NO: 23;
ii) the HFR1 comprising the amino acid sequence set forth in SEQ ID NO: 12, the HFR2 comprising the amino acid sequence set forth in SEQ ID NO: 16, the HFR3 comprising the amino acid sequence set forth in SEQ ID NO: 20, and the HFR4 comprising the amino acid sequence set forth in SEQ ID NO: 24;
iii) the HFR1 comprising the amino acid sequence set forth in SEQ ID NO: 13, the HFR2 comprising the amino acid sequence set forth in SEQ ID NO: 17, the HFR3 comprising the amino acid sequence set forth in SEQ ID NO: 21, and the HFR4 comprising the amino acid sequence set forth in SEQ ID NO: 23; and
vi) the HFR1 comprising the amino acid sequence set forth in SEQ ID NO: 14, the HFR2 comprising the amino acid sequence set forth in SEQ ID NO: 17, the HFR3 comprising the amino acid sequence set forth in SEQ ID NO: 20, and the HFR4 comprising the amino acid sequence set forth in SEQ ID NO: 24.

In certain embodiments, the VH comprises an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 25. For example, the VH may comprise an amino acid sequence set forth in SEQ ID NO: 25.

In certain embodiments, the VH comprises an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 26, SEQ ID NO: 27, SEQ ID NO: 28, or SEQ ID NO: 29. For example, the VH may comprise an amino acid sequence set forth in SEQ ID NO: 26, SEQ ID NO: 27, SEQ ID NO: 28, or SEQ ID NO: 29.

In certain embodiments, the antigen-binding polypeptide includes an antibody or an antigen-binding fragment thereof.

In certain embodiments, the antibody includes a monoclonal antibody, a polyclonal antibody, a dimer, a polymer, a multispecific antibody, an intact antibody, an antibody fragment, a human antibody, a humanized antibody, or a chimeric antibody.

In certain embodiments, the antigen-binding fragment includes a Fab fragment, an Fv fragment, F(ab′)₂, a single-chain Fv (scFv), or a single-domain antibody (VHH).

### Chimeric Antigen Receptor

In one aspect, the present application provides a chimeric antigen receptor (CAR) targeting B7H3, which comprises a targeting moiety, wherein the targeting moiety comprises the aforementioned antigen-binding polypeptide.

In certain embodiments, the targeting moiety includes a VHH.

For example, the targeting moiety comprises a VHH, wherein the VHH may comprise: HCDR1 comprising an amino acid sequence set forth in SEQ ID NO: 1, HCDR2 comprising an amino acid sequence set forth in SEQ ID NO: 4, and HCDR3 comprising an amino acid sequence set forth in SEQ ID NO: 7.

As another example, the targeting moiety comprises a VHH, wherein the VHH may comprise an amino acid sequence set forth in SEQ ID NO: 25.

In certain embodiments, the chimeric antigen receptor comprises a transmembrane domain, wherein the transmembrane domain comprises a transmembrane domain derived from one or more proteins selected from the group consisting of: CD8A, CD8B, CD28, CD3ε (CD3e), 4-1BB, CD4, CD27, CD7, PD-1, TRAC, TRBC, CD3ζ, CTLA-4, LAG-3, CD5, ICOS, OX40, NKG2D, 2B4 (CD244), FcεRIγ, BTLA, CD30, GITR, HVEM, DAP10, CD2, NKG2C, LIGHT, DAP12, CD40L (CD154), TIM1, CD226, DR3, CD45, CD80, CD86, CD9, CD16, CD22, CD33, CD37, CD64, and SLAM.

In certain embodiments, the transmembrane domain comprises a transmembrane domain derived from CD8A.

In certain embodiments, the transmembrane domain comprises an amino acid sequence having at least about 90%, about 91%, about 92%, about 93%, about 94%, about 95%, about 96%, about 97%, about 98%, about 99%, or about 99.5% identity to the amino acid sequence set forth in any one of SEQ ID NO: 42 to SEQ ID NO: 90.

In certain embodiments, the chimeric antigen receptor comprises an intracellular co-stimulatory signaling domain, wherein the intracellular co-stimulatory signaling domain comprises an intracellular co-stimulatory signaling domain derived from one or more proteins selected from the group consisting of: CD28, CD137, CD27, CD2, CD7, CD8A, CD8B, OX40, CD226, DR3, SLAM, CDS, ICAM-1, NKG2D, NKG2C, B7H3, 2B4, FcεRIγ, BTLA, GITR, HVEM, DAP10, DAP12, CD30, CD40, CD40L, TIM1, PD-1, LFA-1, LIGHT, JAML, CD244, CD100, ICOS, CD40, and MyD88.

In certain embodiments, the intracellular co-stimulatory signaling domain is derived from a co-stimulatory signaling domain of 4-1BB.

In certain embodiments, the intracellular co-stimulatory signaling domain comprises an amino acid sequence having at least about 90%, about 91%, about 92%, about 93%, about 94%, about 95%, about 96%, about 97%, about 98%, about 99%, or about 99.5% identity to the amino acid sequence set forth in any one of SEQ ID NO: 91 to SEQ ID NO: 123.

In certain embodiments, the chimeric antigen receptor comprises an intracellular signaling domain, wherein the intracellular signaling domain comprises an intracellular signaling domain derived from one or more proteins selected from the group consisting of: CD3ζ, CD3δ, CD3γ, CD3ε, CD79a, CD79b, FceIγ, FceRIβ, FcγRIIa, bovine leukemia virus gp30, Epstein-Barr virus (EBV) LMP2A, simian immunodeficiency virus PBj14 Nef, DAP10, DAP-12, and a domain comprising at least one ITAM.

In certain embodiments, the intracellular signaling domain comprises a signaling domain derived from CD3ζ.

In certain embodiments, the intracellular signaling domain comprises an amino acid sequence having at least about 90%, about 91%, about 92%, about 93%, about 94%, about 95%, about 96%, about 97%, about 98%, about 99%, or about 99.5% identity to the amino acid sequence set forth in any one of SEQ ID NO: 107, SEQ ID NO: 111, SEQ ID NO: 112, and SEQ ID NO: 124 to SEQ ID NO: 134.

In certain embodiments, the chimeric antigen receptor comprises a hinge region between the targeting moiety and the transmembrane domain, wherein the hinge region comprises a hinge region derived from one or more proteins selected from the group consisting of: CD28, IgG1, IgG4, IgD, 4-1BB, CD4, CD27, CD7, CD8A, PD-1, ICOS, OX40, NKG2D, NKG2C, FcεRIγ, BTLA, GITR, DAP10, TIM1, SLAM, CD30, and LIGHT.

In certain embodiments, the hinge region comprises a hinge region derived from CD8A.

In certain embodiments, the hinge region comprises an amino acid sequence having at least about 90%, about 91%, about 92%, about 93%, about 94%, about 95%, about 96%, about 97%, about 98%, about 99%, or about 99.5% identity to the amino acid sequence set forth in any one of SEQ ID NO: 135 to SEQ ID NO: 156.

In certain embodiments, a non-targeting moiety of the chimeric antigen receptor comprises a hinge region, a transmembrane domain, an intracellular co-stimulatory signaling domain, and an intracellular signaling domain.

In certain embodiments, a non-targeting moiety of the chimeric antigen receptor comprises a transmembrane domain of CD8A molecule, a hinge region of CD8A, an intracellular co-stimulatory signaling domain of 4-1BB, and an intracellular signaling domain of CD3ζ.

For example, the chimeric antigen receptor uses an anti-B7H3 single-domain antibody as an extracellular antigen-binding domain linked to an intracellular signaling domain via a hinge region and a transmembrane domain of the CD8A molecule, the intracellular signaling domain consisting of a 4-1BB intracellular co-stimulatory signaling domain and a CD3ζ intracellular signaling domain.

In certain embodiments, the non-targeting moiety of the chimeric antigen receptor comprises an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 30.

In certain embodiments, the chimeric antigen receptor further comprises a signal peptide fragment, wherein the C-terminus of the signal peptide fragment is linked to the N-terminus of the targeting moiety. For example, the chimeric antigen receptor may include a CAR comprising a signal peptide, an anti-B7H3 VHH, a CD8A hinge domain, a CD8A transmembrane domain, a 4-1BB co-stimulatory domain, and a CD3ζ primary signaling domain.

In certain embodiments, the signal peptide fragment includes a CD8A signal peptide fragment.

In certain embodiments, the signal peptide fragment of the chimeric antigen receptor comprises an amino acid sequence having at least about 90%, about 91%, about 92%, about 93%, about 94%, about 95%, about 96%, about 97%, about 98%, about 99%, or about 99.5% identity to the amino acid sequence set forth in SEQ ID NO: 31.

### Nucleic Acid Molecule, Vector, and Cell

In another aspect, the present application provides one or more isolated nucleic acid molecules encoding the aforementioned antigen-binding polypeptide or the aforementioned chimeric antigen receptor.

In certain embodiments, the isolated nucleic acid molecule comprises an amino acid sequence having at least about 90%, about 91%, about 92%, about 93%, about 94%, about 95%, about 96%, about 97%, about 98%, about 99%, or about 99.5% identity to the nucleotide sequence set forth in any one of SEQ ID NO: 36, SEQ ID NO: 37, SEQ ID NO: 38, and SEQ ID NO: 39.

In another aspect, the present application provides a vector comprising the aforementioned isolated nucleic acid molecule.

In certain embodiments, the vector is an expression vector.

In certain embodiments, the vector is selected from a DNA vector, an RNA vector, a plasmid, a lentiviral vector, an adenoviral vector, an adeno-associated viral vector, and a retroviral vector. For example, the vector may be a lentiviral vector.

In another aspect, the present application provides a cell i) comprising the aforementioned isolated nucleic acid molecule or the aforementioned vector; and/or ii) expressing the aforementioned antigen-binding polypeptide or chimeric antigen receptor.

### Immune Effector Cell

In another aspect, the present application provides an immune effector cell comprising the aforementioned nucleic acid molecule or the aforementioned vector, and/or expressing the aforementioned CAR.

In certain embodiments, the immune effector cell includes a human cell.

In certain embodiments, the immune effector cell includes a T cell, a B cell, a natural killer cell (NK cell), a macrophage, an NKT cell, a monocyte, a dendritic cell, a granulocyte, a lymphocyte, a leukocyte, and/or a peripheral blood mononuclear cell. For example, the immune effector cell may be a T cell. As another example, the immune effector cell may be a human T cell.

In certain embodiments, the immune effector cell includes an autologous or non-autologous immune effector cell.

In certain embodiments, the immune effector cell includes a modified immune effector cell.

In certain embodiments, the modified immune effector cell includes a cell that reduces immune rejection caused by allogeneic cell therapy.

In certain embodiments, the functions of a T cell antigen receptor (TCR) and major histocompatibility complexes (MHCI, MHCII) in the modified immune effector cell are inhibited in a T cell.

In certain embodiments, the modification comprises down-regulation of the expression and/or activity of one or more of immune rejection-related genes.

In certain embodiments, the immune rejection-related gene is selected from one or more of the following groups: TRAC, TRBC, HLA-A, HLA-B, B2M, and CIITA.

In certain embodiments, the immune rejection-related gene is selected from one or more of the following groups: TRAC, TRBC, HLA-A, and HLA-B.

In certain embodiments, the immune rejection-related gene is selected from one or more of the following groups: TRAC, TRBC, and HLA-A.

In certain embodiments, the immune rejection-related gene is selected from one or more of the following groups: TRAC and HLA-A.

In certain embodiments, the expression and/or activity of the TRAC gene and the HLA-A gene in the modified immune effector cell is down-regulated as compared to a corresponding unmodified cell.

In certain embodiments, the expression and/or activity of the CIITA gene in the modified immune effector cell is not down-regulated as compared to the corresponding unmodified cell.

In certain embodiments, the expression and/or activity of the B2M gene in the modified immune effector cell is not down-regulated as compared to the corresponding unmodified cell.

In certain embodiments, the expression and/or activity of the TRAC gene and the HLA-A gene in the modified immune effector cell is down-regulated as compared to a corresponding wild-type cell.

In certain embodiments, the expression and/or activity of the B2M gene in the modified immune effector cell is not down-regulated as compared to the corresponding wild-type cell.

In certain embodiments, the expression and/or activity of the CIITA gene in the modified immune effector cell is not down-regulated as compared to the corresponding wild-type cell.

In certain embodiments, the down-regulation of the expression level and/or activity of the gene includes down-regulating the expression and/or activity of a nucleic acid molecule encoding the gene; and/or down-regulating the expression and/or activity of a protein product encoded by the gene.

In certain embodiments, the modification comprises: gene knockout, gene mutation, and/or gene silencing.

In certain embodiments, the modification comprises knocking out either of two TRAC alleles and knocking out either of two HLA-A alleles in the immune effector cell.

In certain embodiments, the modification comprises knocking out the two TRAC alleles and knocking out either of the two HLA-A alleles in the immune cell.

In certain embodiments, the modification comprises knocking out an exon of the TRAC gene and knocking out an exon of the HLA-A gene in the immune cell.

In certain embodiments, the modification comprises administering to the immune effector cell one or more substances selected from the group consisting of: antisense RNA, siRNA, shRNA, and a CRISPR/Cas9 system.

In certain embodiments, the modification comprises administering to the immune effector cell the CRISPR/Cas9 system.

In certain embodiments, the modification further comprises administering to the immune effector cell sgRNA targeting an exon portion of the TRAC gene.

In certain embodiments, the sgRNA targeting the exon portion of the TRAC gene comprises an amino acid sequence having at least about 90%, about 91%, about 92%, about 93%, about 94%, about 95%, about 96%, about 97%, about 98%, about 99%, or about 99.5% identity to the nucleotide sequence set forth in any one of SEQ ID NO: 157 to SEQ ID NO: 171.

In certain embodiments, the modification comprises administering to the immune effector cell sgRNA targeting an exon portion of the HLA-A gene.

In certain embodiments, the sgRNA targeting the exon portion of the HLA-A gene comprises an amino acid sequence having at least about 90%, about 91%, about 92%, about 93%, about 94%, about 95%, about 96%, about 97%, about 98%, about 99%, or about 99.5% identity to the nucleotide sequence set forth in any one of SEQ ID NO: 172 to SEQ ID NO: 212.

In certain embodiments, the modification further comprises administering to the cell a Cas enzyme.

In certain embodiments, the Cas enzyme includes a Cas9 protein.

In certain embodiments, the antisense RNA comprises an amino acid sequence having at least about 90%, about 91%, about 92%, about 93%, about 94%, about 95%, about 96%, about 97%, about 98%, about 99%, or about 99.5% identity to the nucleotide sequence set forth in any one of SEQ ID NO: 213 to SEQ ID NO: 216.

In certain embodiments, the immune effector cell is an HLA-B homozygous cell. In certain embodiments, the HLA-B homozygote includes HLA-B*40 homozygote, HLA-B*15 homozygote, HLA-B*46 homozygote, HLA-B*13 homozygote, HLA-B*51 homozygote, HLA-B*58 homozygote, HLA-B*07 homozygote, HLA-B*35 homozygote, HLA-B*44 homozygote, HLA-B*52 homozygote, HLA-B*57 homozygote, HLA-B*54 homozygote, and HLA-B*55 homozygote.

In certain embodiments, the immune effector cell is an HLA-A homozygous or heterozygous cell.

In certain embodiments, the HLA-A homozygote or heterozygote includes HLA-A*02 homozygote, HLA-A*11 homozygote, HLA-A*02/A*11 heterozygote, or HLA-A*24 homozygote.

In another aspect, the present application provides a method for preparing an immune effector cell, which comprises introducing the aforementioned nucleic acid molecule or the aforementioned vector into the immune effector cell.

In certain embodiments, the method further comprises: modifying the immune effector cell before/after introducing the aforementioned nucleic acid molecule or the aforementioned vector into the immune effector cell, wherein the modification comprises down-regulation of the expression and/or activity of one or more of immune rejection-related genes.

In certain embodiments, the method comprises: modifying the immune effector cell after introducing the aforementioned nucleic acid molecule or the aforementioned vector into the immune effector cell, wherein the modification comprises down-regulation of the expression and/or activity of one or more of immune rejection-related genes.

For example, the method for preparing an immune effector cell may comprise:
(1) introducing the aforementioned nucleic acid molecule or the aforementioned vector into an immune effector cell; and
(2) modifying the immune effector cell, wherein the modification comprises down-regulation of the expression and/or activity of one or more of immune rejection-related genes.

In certain embodiments, the immune rejection-related gene is selected from one or more of the following groups: TRAC, TRBC, HLA-A, HLA-B, B2M, and CIITA.

In certain embodiments, the expression and/or activity of the TRAC gene and the HLA-A gene in the immune effector cell is down-regulated as compared to the expression and/or activity of a corresponding gene in a corresponding unmodified cell.

In certain embodiments, the expression and/or activity of the CIITA gene is not down-regulated as compared to the expression and/or activity of the corresponding gene in the corresponding unmodified cell.

In certain embodiments, the expression and/or activity of the B2M gene is not down-regulated as compared to the expression and/or activity of the corresponding gene in the corresponding unmodified cell.

In certain embodiments, the expression and/or activity of the TRAC gene and the HLA-A gene in the immune effector cell is down-regulated as compared to a corresponding wild-type cell.

In certain embodiments, the expression and/or activity of the CIITA gene is not down-regulated as compared to the corresponding wild-type cell.

In certain embodiments, the expression and/or activity of the B2M gene is not down-regulated as compared to the corresponding wild-type cell.

In certain embodiments, the down-regulation of the expression level and/or activity of the gene includes down-regulating the expression and/or activity of a nucleic acid molecule encoding the gene; and/or down-regulating the expression and/or activity of a protein product encoded by the gene.

In certain embodiments, the modification comprises: gene knockout, gene mutation, and/or gene silencing.

In certain embodiments, the modification comprises knocking out either of two TRAC alleles and knocking out either of two HLA-A alleles in the immune effector cell.

In certain embodiments, the modification comprises knocking out the two TRAC alleles and knocking out either of the two HLA-A alleles in the immune cell.

In certain embodiments, the modification comprises knocking out an exon of the TRAC gene and knocking out an exon of the HLA-A gene in the immune cell.

In certain embodiments, the modification comprises administering to the immune effector cell one or more substances selected from the group consisting of: antisense RNA, siRNA, shRNA, and a CRISPR/Cas9 system.

In certain embodiments, the modification comprises administering to the immune effector cell the CRISPR/Cas9 system.

In certain embodiments, the modification comprises administering to the immune effector cell sgRNA targeting an exon portion of the TRAC gene.

In certain embodiments, the sgRNA targeting the exon portion of the TRAC gene comprises a nucleotide sequence set forth in any one of SEQ ID NO: 157 to SEQ ID NO: 171.

In certain embodiments, the modification comprises administering to the immune effector cell sgRNA targeting an exon portion of the HLA-A gene.

In certain embodiments, the sgRNA targeting the exon portion of the HLA-A gene comprises an amino acid sequence having at least about 90%, about 91%, about 92%, about 93%, about 94%, about 95%, about 96%, about 97%, about 98%, about 99%, or about 99.5% identity to the nucleotide sequence set forth in any one of SEQ ID NO: 172 to SEQ ID NO: 212.

In certain embodiments, the modification further comprises administering to the cell a Cas enzyme.

In certain embodiments, the Cas enzyme includes a Cas9 protein.

In certain embodiments, the antisense RNA comprises an amino acid sequence having at least about 90%, about 91%, about 92%, about 93%, about 94%, about 95%, about 96%, about 97%, about 98%, about 99%, or about 99.5% identity to the nucleotide sequence set forth in any one of SEQ ID NO: 213 to SEQ ID NO: 216.

In certain embodiments, the immune effector cell includes a human cell.

In certain embodiments, the immune effector cell includes a T cell, a B cell, a natural killer cell (NK cell), a macrophage, an NKT cell, a monocyte, a dendritic cell, a granulocyte, a lymphocyte, a leukocyte, and/or a peripheral blood mononuclear cell. For example, the immune effector cell may be a T cell.

In certain embodiments, the immune effector cell includes an autologous or non-autologous immune effector cell.

In certain embodiments, the cell is an HLA-B homozygous cell.

In certain embodiments, the HLA-B homozygote includes HLA-B*40 homozygote, HLA-B*15 homozygote, HLA-B*46 homozygote, HLA-B*13 homozygote, HLA-B*51 homozygote, HLA-B*58 homozygote, HLA-B*07 homozygote, HLA-B*35 homozygote, HLA-B*44 homozygote, HLA-B*52 homozygote, HLA-B*57 homozygote, HLA-B*54 homozygote, and HLA-B*55 homozygote.

In certain embodiments, the cell is an HLA-A homozygous or heterozygous cell.

In certain embodiments, the HLA-A homozygote or heterozygote includes HLA-A*02 homozygote, HLA-A*11 homozygote, HLA-A*02/A*11 heterozygote, or HLA-A*24 homozygote.

For example, the method for preparing an immune effector cell may comprise:
(1) collecting peripheral blood of healthy people, performing HLA typing assay, selecting typing meeting our requirements, separating PBMCs, adding CD3 magnetic beads according to a proportion for incubation, and sorting CD3⁺ T cells; uniformly mixing CD3/CD28 antibody-coupled magnetic beads, measuring an appropriate amount of magnetic bead suspension according to the calculated amount, adding the magnetic bead suspension into a T cell culture system, activating T cells, and performing overnight culture;
(2) infecting the T cells according to the titer of B7H3 CAR virus;
(3) simultaneously knocking out TRAC and HLA-A genes; and
(4) sorting CD3-negative T cells: adding CD3 magnetic beads according to a proportion, and collecting CD3-T cells (cells not bound to the magnetic beads).

### Use, Pharmaceutical Composition, and Treatment Method

In another aspect, the present application provides use of the aforementioned chimeric antigen receptor, the aforementioned isolated nucleic acid molecule, the aforementioned vector, the aforementioned cell, or the aforementioned immune effector cell in the preparation of a CAR-T cell.

In another aspect, the present application provides a pharmaceutical composition comprising the aforementioned antigen-binding polypeptide, the aforementioned chimeric antigen receptor, the aforementioned isolated nucleic acid molecule, the aforementioned vector, the aforementioned cell, and/or the aforementioned immune effector cell, and optionally a pharmaceutically acceptable carrier.

For example, the pharmaceutical composition may include: buffers, such as neutral buffered saline, phosphate buffered saline, and the like; sugars, such as glucose, mannose, sucrose, dextran, or mannitol; proteins; polypeptides or amino acids such as glycine; antioxidants; chelating agents, such as EDTA or glutathione; adjuvants, such as aluminum hydroxide; and preservatives.

For example, the pharmaceutical composition comprises the aforementioned immune effector cell and optionally a pharmaceutically acceptable carrier.

In another aspect, the present application provides use of the aforementioned antigen-binding polypeptide, the aforementioned antigen chimeric receptor, the aforementioned isolated nucleic acid molecule, the aforementioned vector, the aforementioned cell, the aforementioned immune effector cell, and/or the aforementioned pharmaceutical composition in the treatment of a disease or disorder associated with the expression of B7H3.

In certain embodiments, the disease or disorder associated with the expression of B7H3 includes a disease or disorder associated with up-regulation of the expression of B7H3.

In certain embodiments, the disease or disorder associated with the expression of B7H3 includes cancer.

In certain embodiments, the cancer includes adrenocortical carcinoma, bladder cancer, breast cancer, cholangiocarcinoma, colorectal cancer, lymphoma, esophageal cancer, brain glioma, head and neck squamous cell carcinoma, kidney cancer, liver cancer, lung cancer, ovarian cancer, pancreatic cancer, prostate cancer, sarcoma, melanoma, gastric cancer, thymus cancer, or endometrial cancer.

In another aspect, the present application provides use of the aforementioned antigen-binding polypeptide, the aforementioned antigen chimeric receptor, the aforementioned isolated nucleic acid molecule, the aforementioned vector, the aforementioned cell, the aforementioned immune effector cell, and/or the aforementioned pharmaceutical composition in the preparation of a medicament for treating a disease or disorder associated with the expression of B7H3.

In certain embodiments, the disease or disorder associated with the expression of B7H3 includes a disease or disorder associated with up-regulation of the expression of B7H3.

In certain embodiments, the disease or disorder associated with the expression of B7H3 includes cancer.

In certain embodiments, the cancer includes adrenocortical carcinoma, bladder cancer, breast cancer, cholangiocarcinoma, colorectal cancer, lymphoma, esophageal cancer, brain glioma, head and neck squamous cell carcinoma, kidney cancer, liver cancer, lung cancer, ovarian cancer, pancreatic cancer, prostate cancer, sarcoma, melanoma, gastric cancer, thymus cancer, or endometrial cancer.

In another aspect, the present application provides a method for preventing or treating a disease or disorder associated with the expression of B7H3, comprising administering to a subject in need thereof an effective amount of the aforementioned antigen-binding polypeptide, the aforementioned antigen chimeric receptor, the aforementioned isolated nucleic acid molecule, the aforementioned vector, the aforementioned cell, the aforementioned immune effector cell, and/or the aforementioned pharmaceutical composition.

In certain embodiments, the disease or disorder associated with the expression of B7H3 includes a disease or disorder associated with up-regulation of the expression of B7H3.

In certain embodiments, the disease or disorder associated with the expression of B7H3 includes cancer.

In certain embodiments, the cancer includes adrenocortical carcinoma, bladder cancer, breast cancer, cholangiocarcinoma, colorectal cancer, lymphoma, esophageal cancer, brain glioma, head and neck squamous cell carcinoma, kidney cancer, liver cancer, lung cancer, ovarian cancer, pancreatic cancer, prostate cancer, sarcoma, melanoma, gastric cancer, thymus cancer, or endometrial cancer.

### Modified Immune Effector Cell

In another aspect, the present application provides a modified immune effector cell, wherein the expression and/or activity of TRAC gene and HLA-A gene is down-regulated, the expression and/or activity of B2M gene is not down-regulated, and the expression and/or activity of CIITA gene is not down-regulated as compared to the expression and/or activity of corresponding genes in a corresponding unmodified cell; and the HLA-B typing of the modified immune effector cell is matched with the HLA-B typing of a subject.

In certain embodiments, the modified immune effector cell is HLA-B heterozygote and is consistent with two alleles of HLA-B of the subject, or the modified immune effector cell is HLA-B homozygote and is consistent with one of the alleles of HLA-B of the subject.

In certain embodiments, the HLA-B homozygote includes HLA-B*40 homozygote, HLA-B*15 homozygote, HLA-B*46 homozygote, HLA-B*13 homozygote, HLA-B*51 homozygote, HLA-B*58 homozygote, HLA-B*07 homozygote, HLA-B*35 homozygote, HLA-B*44 homozygote, HLA-B*52 homozygote, HLA-B*57 homozygote, HLA-B*54 homozygote, and HLA-B*55 homozygote.

In certain embodiments, the modification enables the expression and/or activity of two genes to be down-regulated, wherein the two genes consist of TRAC gene and HLA-A gene.

In certain embodiments, the expression and/or activity of the TRAC gene and the HLA-A gene is down-regulated, the expression and/or activity of the B2M gene is not down-regulated, and the expression and/or activity of the CIITA gene is not down-regulated as compared to a corresponding wild-type cell.

In certain embodiments, the expression and/or activity of two genes is down-regulated as compared to the corresponding wild-type cell, wherein the two genes consist of TRAC gene and HLA-A gene.

In certain embodiments, the down-regulation of the expression level and/or activity of the gene includes down-regulating the expression and/or activity of a nucleic acid molecule encoding the gene; and/or down-regulating the expression and/or activity of a protein product encoded by the gene.

In certain embodiments, the modification comprises: gene mutation and/or gene silencing.

In certain embodiments, the modification comprises administering to the immune effector cell one or more substances selected from the group consisting of: antisense RNA, siRNA, shRNA, and a CRISPR/Cas9 system.

In certain embodiments, the modification comprises administering to the immune effector cell the CRISPR/Cas9 system.

In certain embodiments, the modification comprises administering to the immune effector cell sgRNA targeting an exon portion of the HLA-A gene.

In certain embodiments, the sgRNA targeting the exon portion of the HLA-A gene comprises a nucleotide sequence set forth in any one of SEQ ID NO: 172 to SEQ ID NO: 212.

In certain embodiments, the modification further comprises administering to the immune effector cell sgRNA targeting an exon portion of the TRAC gene.

In certain embodiments, the sgRNA targeting the exon portion of the TRAC gene comprises a nucleotide sequence set forth in any one of SEQ ID NO: 157 to SEQ ID NO: 171.

In certain embodiments, the modification further comprises administering to the cell a Cas enzyme.

In certain embodiments, the Cas enzyme includes a Cas9 protein.

In certain embodiments, the antisense RNA comprises a nucleotide sequence set forth in any one of SEQ ID NO: 213 to SEQ ID NO: 216.

In certain embodiments, the modified immune effector cell expresses a CAR.

In certain embodiments, the CAR comprises an antigen-binding domain, a hinge region, a transmembrane domain, an intracellular co-stimulatory signaling domain, and an intracellular signaling domain.

In certain embodiments, the antigen-binding domain specifically binds to a tumor antigen.

In certain embodiments, the tumor antigen is selected from the group consisting of: CD19, CD20, CD22, CD33, BCMA, IL13Ra2, EGFR, Her2, GD2, and B7H3.

In certain embodiments, the antigen-binding domain is selected from the group consisting of: a monoclonal antibody, a polyclonal antibody, a dimer, a polymer, a multispecific antibody, an intact antibody, an antibody fragment, a human antibody, a humanized antibody, a chimeric antibody, an Fv fragment, F(ab′)2, a single-chain Fv(scFv), and a single-domain antibody (VHH).

In certain embodiments, the transmembrane domain comprises a transmembrane domain derived from one or more proteins selected from the group consisting of: CD8A, CD8B, CD28, CD38 (CD3e), 4-1BB, CD4, CD27, CD7, PD-1, TRAC, TRBC, CD3ζ, CTLA-4, LAG-3, CD5, ICOS, OX40, NKG2D, 2B4 (CD244), FcεRIγ, BTLA, CD30, GITR, HVEM, DAP10, CD2, NKG2C, LIGHT, DAP12, CD40L (CD154), TIM1, CD226, DR3, CD45, CD80, CD86, CD9, CD16, CD22, CD33, CD37, CD64, and SLAM.

In certain embodiments, the intracellular co-stimulatory signaling domain comprises an intracellular co-stimulatory signaling domain derived from one or more proteins selected from the group consisting of: CD28, CD137, CD27, CD2, CD7, CD8A, CD8B, OX40, CD226, DR3, SLAM, CDS, ICAM-1, NKG2D, NKG2C, B7H3, 2B4, FcεRIγ, BTLA, GITR, HVEM, DAP10, DAP12, CD30, CD40, CD40L, TIM1, PD-1, LFA-1, LIGHT, JAML, CD244, CD100, ICOS, CD40, and MyD88.

In certain embodiments, the intracellular signaling domain comprises an intracellular signaling domain derived from one or more proteins selected from the group consisting of: CD3ζ, CD3δ, CD3γ, CD3ε, CD79a, CD79b, FceRIγ, FceRIβ, FcγRIIa, bovine leukemia virus gp30, Epstein-Barr virus (EBV) LMP2A, simian immunodeficiency virus PBj14 Nef, DAP10, DAP-12, and a domain comprising at least one ITAM.

In certain embodiments, the hinge region comprises a hinge region derived from one or more proteins selected from the group consisting of: CD28, IgG1, IgG4, IgD, 4-1BB, CD4, CD27, CD7, CD8A, PD-1, ICOS, OX40, NKG2D, NKG2C, FcεRIγ, BTLA, GITR, DAP10, TIM1, SLAM, CD30, and LIGHT.

In certain embodiments, the CAR further comprises a signal peptide fragment, wherein the C-terminus of the signal peptide fragment is linked to the N-terminus of the targeting moiety.

In certain embodiments, the signal peptide fragment includes a CD8A signal peptide fragment.

In certain embodiments, the immune effector cell includes a human cell.

In certain embodiments, the immune effector cell includes a T cell, a B cell, a natural killer cell (NK cell), a macrophage, an NKT cell, a monocyte, a dendritic cell, a granulocyte, a lymphocyte, a leukocyte, and/or a peripheral blood mononuclear cell.

In certain embodiments, the immune effector cell includes a non-autologous immune effector cell.

In another aspect, the present application provides a method for preparing the aforementioned modified immune effector cell, comprising the following steps:
1) selecting an immune effector cell that matches HLA-B typing of a subject; and
2) down-regulating the expression and/or activity of TRAC gene and HLA-A gene in the immune effector cell, not down-regulating the expression and/or activity of B2M gene, and not down-regulating the expression and/or activity of CIITA gene as compared to the expression and/or activity of corresponding genes in a corresponding unmodified cell.

In certain embodiments, the modified immune effector cell is HLA-B heterozygote and is consistent with two alleles of HLA-B of the subject, or the modified immune effector cell is HLA-B homozygote and is consistent with one of the alleles of HLA-B of the subject.

In certain embodiments, the HLA-B homozygote includes HLA-B*40 homozygote, HLA-B*15 homozygote, HLA-B*46 homozygote, HLA-B*13 homozygote, HLA-B*51 homozygote, HLA-B*58 homozygote, HLA-B*07 homozygote, HLA-B*35 homozygote, HLA-B*44 homozygote, HLA-B*52 homozygote, HLA-B*57 homozygote, HLA-B*54 homozygote, and HLA-B*55 homozygote.

In certain embodiments, the modification enables the expression and/or activity of two genes to be down-regulated, wherein the two genes consist of TRAC gene and HLA-A gene.

In certain embodiments, the expression and/or activity of the TRAC gene and the HLA-A gene is down-regulated, the expression and/or activity of the B2M gene is not down-regulated, and the expression and/or activity of the CIITA gene is not down-regulated as compared to a corresponding wild-type cell.

In certain embodiments, the expression and/or activity of two genes is down-regulated as compared to the corresponding wild-type cell, wherein the two genes consist of TRAC gene and HLA-A gene.

In certain embodiments, the down-regulation of the expression level and/or activity of the gene includes down-regulating the expression and/or activity of a nucleic acid molecule encoding the gene; and/or down-regulating the expression and/or activity of a protein product encoded by the gene.

In certain embodiments, the modification comprises: gene mutation and/or gene silencing.

In certain embodiments, the modification comprises administering to the immune effector cell one or more substances selected from the group consisting of: antisense RNA, siRNA, shRNA, and a CRISPR/Cas9 system.

In certain embodiments, the modification comprises administering to the immune effector cell the CRISPR/Cas9 system.

In certain embodiments, the modification comprises administering to the immune effector cell sgRNA targeting an exon portion of the HLA-A gene.

In certain embodiments, the sgRNA targeting the exon portion of the HLA-A gene comprises a nucleotide sequence set forth in any one of SEQ ID NO: 172 to SEQ ID NO: 212.

In certain embodiments, the modification comprises administering to the immune effector cell sgRNA targeting an exon portion of the TRAC gene.

In certain embodiments, the sgRNA targeting the exon portion of the TRAC gene comprises a nucleotide sequence set forth in any one of SEQ ID NO: 157 to SEQ ID NO: 171.

In certain embodiments, the modification further comprises administering to the cell a Cas enzyme.

In certain embodiments, the Cas enzyme includes a Cas9 protein.

In certain embodiments, the antisense RNA comprises a nucleotide sequence set forth in any one of SEQ ID NO: 213 to SEQ ID NO: 216.

In certain embodiments, the immune effector cell includes a human cell.

In certain embodiments, the immune effector cell includes a T cell, a B cell, a natural killer cell (NK cell), a macrophage, an NKT cell, a monocyte, a dendritic cell, a granulocyte, a lymphocyte, a leukocyte, and/or a peripheral blood mononuclear cell.

In certain embodiments, the immune effector cell includes a non-autologous immune effector cell.

In another aspect, the present application provides a composition comprising the aforementioned modified immune effector cell and a pharmaceutically acceptable carrier.

In another aspect, the present application provides use of the aforementioned modified immune effector cell in the preparation of a CAR-T cell.

In another aspect, the present application provides use of the aforementioned modified immune effector cell in the preparation of a medicament for allogeneic therapy.

In another aspect, the present application provides use of the aforementioned modified immune effector cell in the preparation of a medicament for treating a tumor.

In certain embodiments, the tumor includes a solid tumor and a non-solid tumor.

In certain embodiments, the tumor is selected from the group consisting of: liver cancer, gastric cancer, lung cancer, breast cancer, non-small cell lung cancer, B-lymphomas, Hodgkin's lymphoma, gliomas, chronic myelogenous leukemia, and acute myeloid leukemia.

The present application further discloses the following embodiments:

128. A modified immune effector cell, wherein the expression and/or activity of TRAC gene and HLA-A gene is down-regulated, the expression and/or activity of B2M gene is not down-regulated, and the expression and/or activity of CIITA gene is not down-regulated as compared to the expression and/or activity of corresponding genes in a corresponding unmodified cell; and the HLA-B typing of the modified immune effector cell is matched with the HLA-B typing of a subject.
129. The modified immune effector cell according to claim 128, wherein the modified immune effector cell is HLA-B heterozygote and is consistent with two alleles of HLA-B of the subject, or the modified immune effector cell is HLA-B homozygote and is consistent with one of the alleles of HLA-B of the subject.
130. The modified immune effector cell according to claim 129, wherein the HLA-B homozygote comprises HLA-B*40 homozygote, HLA-B*15 homozygote, HLA-B*46 homozygote, HLA-B*13 homozygote, HLA-B*51 homozygote, HLA-B*58 homozygote, HLA-B*07 homozygote, HLA-B*35 homozygote, HLA-B*44 homozygote, HLA-B*52 homozygote, HLA-B*57 homozygote, HLA-B*54 homozygote, and HLA-B*55 homozygote.
131. The modified immune effector cell according to any one of claims 128-130, wherein the modification enables the expression and/or activity of two genes to be down-regulated, wherein the two genes consist of TRAC gene and HLA-A gene.
132. The modified immune effector cell according to any one of claims 128-131, wherein the expression and/or activity of the TRAC gene and the HLA-A gene is down-regulated, the expression and/or activity of the B2M gene is not down-regulated, and the expression and/or activity of the CIITA gene is not down-regulated as compared to a corresponding wild-type cell.
133. The modified immune effector cell according to any one of claims 128-132, wherein the expression and/or activity of two genes is down-regulated as compared to the corresponding wild-type cell, wherein the two genes consist of TRAC gene and HLA-A gene.
134. The modified immune effector cell according to any one of claims 128-133, wherein the down-regulation of the expression level and/or activity of the gene comprises down-regulating the expression and/or activity of a nucleic acid molecule encoding the gene; and/or down-regulating the expression and/or activity of a protein product encoded by the gene.
135. The modified immune effector cell according to any one of claims 128-134, wherein the modification comprises: gene mutation and/or gene silencing.
136. The modified immune effector cell according to any one of claims 128-135, wherein the modification comprises administering to the immune effector cell one or more substances selected from the group consisting of: antisense RNA, siRNA, shRNA, and a CRISPR/Cas9 system.
137. The modified immune effector cell according to any one of claims 128-136, wherein the modification comprises administering to the immune effector cell the CRISPR/Cas9 system.
138. The modified immune effector cell according to claim 137, wherein the modification comprises administering to the immune effector cell sgRNA targeting an exon portion of the HLA-A gene.
139. The modified immune effector cell according to claim 138, wherein the sgRNA targeting the exon portion of the HLA-A gene comprises a nucleotide sequence set forth in any one of SEQ ID NO: 172 to SEQ ID NO: 212.
140. The modified immune effector cell according to any one of claims 137-139, wherein the modification further comprises administering to the immune effector cell sgRNA targeting an exon portion of the TRAC gene.
141. The modified immune effector cell according to claim 140, wherein the sgRNA targeting the exon portion of the TRAC gene comprises a nucleotide sequence set forth in any one of SEQ ID NO: 157 to SEQ ID NO: 171.
142. The modified immune effector cell according to any one of claims 137-141, wherein the modification further comprises administering to the cell a Cas enzyme.
143. The modified immune effector cell according to claim 142, wherein the Cas enzyme comprises a Cas9 protein.
144. The modified immune effector cell according to claim 136, wherein the antisense RNA comprises a nucleotide sequence set forth in any one of SEQ ID NO: 213 to SEQ ID NO: 216.
145. The modified immune effector cell according to any one of claims 128-144, wherein the immune effector cell expresses a CAR.
146. The modified immune effector cell according to claim 145, wherein the CAR comprises an antigen-binding domain, a hinge region, a transmembrane domain, an intracellular co-stimulatory signaling domain, and an intracellular signaling domain.
147. The modified immune effector cell according to claim 146, wherein the antigen-binding domain specifically binds to a tumor antigen.
148. The modified immune effector cell according to claim 147, wherein the tumor antigen is selected from the group consisting of: CD19, CD20, CD22, CD33, BCMA, IL13Ra2, EGFR, Her2, GD2, and B7H3.
149. The modified immune effector cell according to any one of claims 146-148, wherein the antigen-binding domain is selected from the group consisting of: a monoclonal antibody, a polyclonal antibody, a dimer, a polymer, a multispecific antibody, an intact antibody, an antibody fragment, a human antibody, a humanized antibody, a chimeric antibody, an Fv fragment, F(ab′)2, a single-chain Fv(scFv), and a single-domain antibody (VHH).
150. The modified immune effector cell according to any one of claims 146-149, wherein the transmembrane domain comprises a transmembrane domain derived from one or more proteins selected from the group consisting of: CD8A, CD8B, CD28, CD3ε (CD3e), 4-1BB, CD4, CD27, CD7, PD-1, TRAC, TRBC, CD3ζ, CTLA-4, LAG-3, CD5, ICOS, OX40, NKG2D, 2B4 (CD244), FcεRIγ, BTLA, CD30, GITR, HVEM, DAP10, CD2, NKG2C, LIGHT, DAP12, CD40L (CD154), TIM1, CD226, DR3, CD45, CD80, CD86, CD9, CD16, CD22, CD33, CD37, CD64, and SLAM.
151. The modified immune effector cell according to any one of claims 146-150, wherein the intracellular co-stimulatory signaling domain comprises an intracellular co-stimulatory signaling domain derived from one or more proteins selected from the group consisting of: CD28, CD137, CD27, CD2, CD7, CD8A, CD8B, OX40, CD226, DR3, SLAM, CDS, ICAM-1, NKG2D, NKG2C, B7H3, 2B4, FcεRIγ, BTLA, GITR, HVEM, DAP10, DAP12, CD30, CD40, CD40L, TIM1, PD-1, LFA-1, LIGHT, JAML, CD244, CD100, ICOS, CD40, and MyD88.
152. The modified immune effector cell according to any one of claims 146-151, wherein the intracellular signaling domain comprises an intracellular signaling domain derived from one or more proteins selected from the group consisting of: CD3ζ, CD3δ, CD3γ, CD3ε, CD79a, CD79b, FceRIγ, FceRIβ, FcγRIIa, bovine leukemia virus gp30, Epstein-Barr virus (EBV) LMP2A, simian immunodeficiency virus PBj14 Nef, DAP10, DAP-12, and a domain comprising at least one ITAM.
153. The modified immune effector cell according to any one of claims 146-152, wherein the hinge region comprises a hinge region derived from one or more proteins selected from the group consisting of: CD28, IgG1, IgG4, IgD, 4-1BB, CD4, CD27, CD7, CD8A, PD-1, ICOS, OX40, NKG2D, NKG2C, FcεRIγ, BTLA, GITR, DAP10, TIM1, SLAM, CD30, and LIGHT.
154. The modified immune effector cell according to any one of claims 146-153, wherein the CAR further comprises a signal peptide fragment, and the C-terminus of the signal peptide fragment is linked to the N-terminus of a targeting moiety.
155. The modified immune effector cell according to any one of claims 146-154, wherein the signal peptide fragment comprises a CD8A signal peptide fragment.
156. The modified immune effector cell according to any one of claims 128-155, wherein the immune effector cell comprises a human cell.
157. The modified immune effector cell according to any one of claims 128-156, wherein the immune effector cell comprises a T cell, a B cell, a natural killer cell (NK cell), a macrophage, an NKT cell, a monocyte, a dendritic cell, a granulocyte, a lymphocyte, a leukocyte, and/or a peripheral blood mononuclear cell.
158. The method according to any one of claims 128-157, wherein the immune effector cell comprises a non-autologous immune effector cell.
159. A method for preparing the modified immune effector cell according to any one of claims 128-158, comprising the following steps:
1) selecting an immune effector cell that matches HLA-B typing of a subject; and
2) down-regulating the expression and/or activity of TRAC gene and HLA-A gene in the immune effector cell, not down-regulating the expression and/or activity of B2M gene, and not down-regulating the expression and/or activity of CIITA gene as compared to the expression and/or activity of corresponding genes in a corresponding unmodified cell.
   160. The method according to claim 159, wherein the modified immune effector cell is HLA-B heterozygote and is consistent with two alleles of HLA-B of the subject, or the modified immune effector cell is HLA-B homozygote and is consistent with one of the alleles of HLA-B of the subject.
   161. The method according to claim 160, wherein the HLA-B homozygote comprises HLA-B*40 homozygote, HLA-B*15 homozygote, HLA-B*46 homozygote, HLA-B*13 homozygote, HLA-B*51 homozygote, HLA-B*58 homozygote, HLA-B*07 homozygote, HLA-B*35 homozygote, HLA-B*44 homozygote, HLA-B*52 homozygote, HLA-B*57 homozygote, HLA-B*54 homozygote, and HLA-B*55 homozygote.
   162. The method according to any one of claims 159-161, wherein the modification enables the expression and/or activity of two genes to be down-regulated, wherein the two genes consist of TRAC gene and HLA-A gene.
   163. The method according to any one of claims 159-162, wherein the expression and/or activity of the TRAC gene and the HLA-A gene is down-regulated, the expression and/or activity of the B2M gene is not down-regulated, and the expression and/or activity of the CIITA gene is not down-regulated as compared to a corresponding wild-type cell.
   164. The method according to any one of claims 159-163, wherein the expression and/or activity of two genes is down-regulated as compared to the corresponding wild-type cell, wherein the two genes consist of TRAC gene and HLA-A gene.
   165. The method according to any one of claims 159-164, wherein the down-regulation of the expression level and/or activity of the gene comprises down-regulating the expression and/or activity of a nucleic acid molecule encoding the gene; and/or down-regulating the expression and/or activity of a protein product encoded by the gene.
   166. The method according to any one of claims 159-165, wherein the modification comprises: gene mutation and/or gene silencing.
   167. The method according to any one of claims 159-166, wherein the modification comprises administering to the immune effector cell one or more substances selected from the group consisting of: antisense RNA, siRNA, shRNA, and a CRISPR/Cas9 system.
   168. The method according to any one of claims 159-167, wherein the modification comprises administering to the immune effector cell the CRISPR/Cas9 system.
   169. The method according to claim 168, wherein the modification comprises administering to the immune effector cell sgRNA targeting an exon portion of the HLA-A gene.
   170. The method according to claim 169, wherein the sgRNA targeting the exon portion of the HLA-A gene comprises a nucleotide sequence set forth in any one of SEQ ID NO: 172 to SEQ ID NO: 212.
   171. The method according to any one of claims 168-171, wherein the modification comprises administering to the immune effector cell sgRNA targeting an exon portion of the TRAC gene.
   172. The method according to claim 171, wherein the sgRNA targeting the exon portion of the TRAC gene comprises a nucleotide sequence set forth in any one of SEQ ID NO: 157 to SEQ ID NO: 171.
   173. The method according to any one of claims 168-172, wherein the modification further comprises administering to the cell a Cas enzyme.
   174. The method according to claim 173, wherein the Cas enzyme comprises a Cas9 protein.
   175. The method according to claim 167, wherein the antisense RNA comprises a nucleotide sequence set forth in any one of SEQ ID NO: 213 to SEQ ID NO: 216.
   176. The method according to any one of claims 159-175, wherein the immune effector cell comprises a human cell.
   177. The method according to any one of claims 159-176, wherein the immune effector cell comprises a T cell, a B cell, a natural killer cell (NK cell), a macrophage, an NKT cell, a monocyte, a dendritic cell, a granulocyte, a lymphocyte, a leukocyte, and/or a peripheral blood mononuclear cell.
   178. The method according to any one of claims 159-177, wherein the immune effector cell comprises a non-autologous immune effector cell.
   179. A composition comprising the modified immune effector cell according to any one of claims 128-158 and a pharmaceutically acceptable carrier.
   180. Use of the modified immune effector cell according to any one of claims 128-158 in the preparation of a CAR-T cell.
   181. Use of the modified immune effector cell according to any one of claims 128-158 in the preparation of a medicament for allogeneic therapy.
   182. Use of the immune effector cell according to any one of claims 128-158 in the preparation of a medicament for treating a tumor.
   183. The use according to claim 182, wherein the tumor comprises a solid tumor and a non-solid tumor.
   184. The use according to any one of claims 182-183, wherein the tumor is selected from the group consisting of: liver cancer, gastric cancer, lung cancer, breast cancer, non-small cell lung cancer, B-lymphomas, Hodgkin's lymphoma, gliomas, chronic myelogenous leukemia, and acute myeloid leukemia.

Without being bound by any theory, the following examples are intended only to illustrate the chimeric antigen receptor, immune effector cell, preparation method, use, etc., of the present application, and are not intended to limit the scope of the present application.

### EXAMPLES

### Example 1

### 1.1 Affinity Assay of Single-Domain Antibodies

The B7H3-Fc recombinant protein was immobilized to a CM5 chip with 10 mM acetate buffer, and the ability of each of the above-prepared single-domain antibodies obtained by screening to bind to the B7H3-Fc recombinant protein was measured with each of the single-domain antibodies as a mobile phase.

### (1) Reagent Preparation

Running reagent: containing 10 mM N-(2-hydroxyethyl)piperazine-N-2 sulfonic acid (HEPES), 150 mM sodium chloride (NaCl), 3 mM ethylenediaminetetraacetic acid (EDTA), and 0.005% Tween-20, pH adjusted to 7.4.

A human IgG (Fc) capture kit comprising a mouse anti-human IgG (Fc) antibody, an immobilization reagent (sodium acetate, pH 5.0), and a regeneration reagent (magnesium chloride).

An amino coupling kit comprising N-hydroxysuccinimide (NHS), 1-ethyl-(3-dimethylaminopropyl)carbodiimide hydrochloride (EDC), and ethanolamine (pH 8.5). To each tube of EDC and NHS was added 10 mL of deionized water, and the mixed solutions were aliquoted and stored at −18° C. or lower, with a two-month shelf life.

### (2) Chip Preparation

The mouse anti-human IgG (Fc) antibody was diluted with the immobilization reagent (sodium acetate, pH 5.0): 950 μL of the immobilization reagent was added to 50 μL of the mouse anti-human IgG (Fc) antibody. The dilution was used for immobilization in eight channels. First, the surface of the CM5 chip was activated for 360 s with EDC and NHS at a flow rate of 10 μL/min. Then, the mouse anti-human IgG (Fc) antibody was injected into the channels (channels 1-8, Fc1,2) at a flow rate of 10 μL/min for about 360 s, with the level of immobilization at about 7000 to 14,000 RU. Finally, the chip was blocked with ethanolamine at 10 μL/min for 420 s.

### (3) Buffer Exchange

Buffer exchange was performed for human B7H3 protein using a desalting column and the corresponding running reagent, and the concentration of the sample after the exchange was determined.

### (4) Ligand Capture

The antibody was diluted to 10 μg/mL with the running reagent, and the dilution was injected into the experimental channels (Fc2) for human IgG (Fc) capture at a flow rate of 10 μL/min at about 300 RU. The reference channels (Fc1) did not require ligand capture.

### (5) Analyte Multicycle Analysis

The human B7H3 protein was diluted 2-fold with the running reagent. The diluted human B7H3 protein was injected into the experimental channels and the reference channels in sequence at a flow rate of 30 μL/min, and corresponding periods of association and dissociation were allowed. The association and dissociation steps were all performed in the running reagent. After each concentration analysis, the chip needed to be regenerated with magnesium chloride at a flow rate of 20 μL/min for 30 s to wash away the ligand and undissociated analyte. For the next concentration analysis, the experimental channels needed to recapture the same amount of ligand.

### (6) Data Analysis

A KD value was calculated for each sample using Biacore 8K analysis software Biacore Insight Evaluation Software. The reference channels (Fc1) were used for background subtraction.

The results are shown in Table 1. The B7H3 single-domain antibodies 1A5 and 1G7 and humanized antibodies thereof of the present application all have relatively high affinity for the human B7H3 protein.

TABLE 1
The results of the binding of single-domain
antibodies to B7H3-Fc recombinant protein

| | Ka (M⁻¹s⁻¹) | Kd (s⁻¹) | KD (M) |
|---|---|---|---|
| 1A5 | 6.09 × 10⁵ | 0.004503 | 7.394 × 10⁻⁹ |
| 1G7 | 1.150 × 10⁶ | 0.006182 | 5.375 × 10⁻⁹ |

### 1.2 Affinity Curves of Antibodies

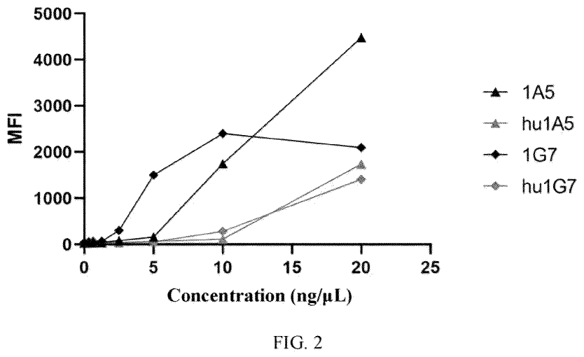

Recombinant antibodies with different concentrations were incubated with U251 cells (expressing B7H3), and the binding of the antibodies to the cell surface was assayed by the mean fluorescence intensity (MFI) of the cells. From the results shown in FIG. 2, the 1A5 and 1G7 antibodies and the humanized antibodies thereof can effectively bind to the surface of the U251 cells.

### 1.3 ADCC Function of Antibodies

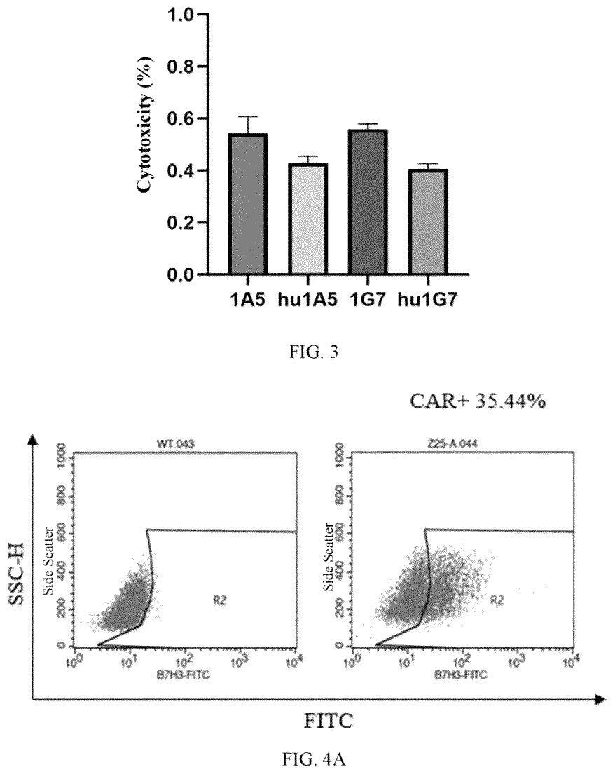

The equivalent amount of NK cells and U251-LG cells were co-cultured, 200 ng/μL of recombinant antibody was added thereto, and a cytotoxicity effect mediated by the antibody was assayed after co-culturing for 24 h. From the results shown in FIG. 3, the 1A5 and 1G7 antibodies and the humanized antibodies thereof can effectively kill the U251-LG tumor cells by ADCC.

### 1.4 Design of Anti-B7H3 Chimeric Antigen Receptor (CAR)

The anti-B7H3 CAR structure comprised: a B7H3 antigen-binding region (derived from an anti-B7H3 single-domain antibody and having an amino acid sequence set forth in SEQ ID NO: 28), a CD8A extracellular hinge region, a CD8A transmembrane region, a 4-1BB intracellular co-stimulatory domain, and a CD3ζ activation signal domain. The amino acid sequence of the non-antigen-binding domain of anti-B7H3 CAR is set forth in SEQ ID NO: 34, and the nucleotide sequence is set forth in SEQ ID NO: 38.

### 1.5 Construction of Anti-B7H3 CAR Lentiviral Vector

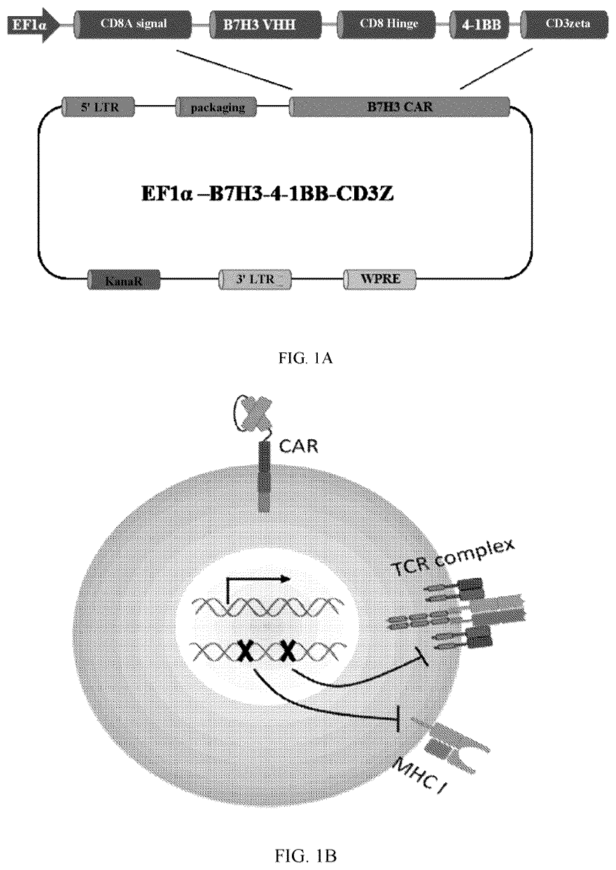

According to the sequence information on B7H3 and the structure of the CAR vector, an anti-B7H3 CAR lentiviral expression vector was constructed, with the vector schematic shown in FIG. 1. The optimization was performed: a commercial lentiviral expression vector pCDH-CMV-MCS-EF1-copGFP was selected as a backbone, and element modification was performed on the basis of the vector. First, an ampicillin resistance gene β-lactamase on the vector was replaced with aminoglycoside phosphotransferase derived from Tn5 to enable the vector to have kanamycin resistance. Secondly, we deleted the CMV promoter and its adjacent downstream multiple cloning site, which were potentially threatening in vivo applications. Finally, the copGFP gene that was started to express by the EF1 promoter in the original vector was deleted, a Sall enzyme digestion site was retained, and a SmaI enzyme digestion site was added to the 5′ end of Sall for vector construction to form a final target vector. The added Smal enzyme digestion site was a single enzyme digestion site of the final target vector, and other sequence portions of the vector did not have the enzyme digestion site. After the optimization, the chimeric antigen receptor lentiviral expression vector was constructed, and lentiviral packaging was performed after confirming that the sequence was correct by Sanger sequencing.

### Example 2

### 2.1 Design of Guide RNA

Corresponding gene sequences were searched and downloaded through a website https://www.ncbi.nlm.nih.gov/, the gene sequences were opened by using SnapGene software, and sgRNAs could be designed on different exons of target genes. The sgRNA of the CRISPR/Cas9 system used in this example was designed following a non-restrictive principle of: 5′-NNN(20)-NGG-3′, NGG being referred to as a protospacer adjacent motif (PAM), wherein N represented A, T, C, or G. Since many sgRNAs could be designed on the same exon and the sgRNA composed of 20 nucleotide sequences could repeatedly occur in a genome, the design and evaluation of sgRNAs were performed using the website http://crispr.cos.uni-heidelberg.de. An exon sequence was pasted to the website, and the sgRNAs were designed and subjected to a prediction evaluation. The higher the score in the evaluation was, the higher the editing efficiency and the lower the off-target risk could exist. The sgRNAs with higher scores were selected for assay. The sgRNAs targeting the TRAC gene were set forth in SEQ ID NO: 157 to SEQ ID NO: 171, the sgRNAs targeting the HLA-A02 gene were set forth in SEQ ID NO: 172 to SEQ ID NO: 193, the sgRNAs targeting the HLA-A11 gene were set forth in SEQ ID NO: 194 to SEQ ID NO: 204, and the sgRNAs targeting the HLA-A24 gene were set forth in SEQ ID NO: 205 to SEQ ID NO: 212, which were synthesized by GenScript Biotech Corporation.

### 2.2 Donor Selection

HLA-B homozygotes which matched with HLA-B typing of a receptor were selected based on the HLA-B typing of the receptor.

First, the donor source was based on HLA-B homozygotes in the population, and one of the alleles of HLA-B in the patient was consistent with the HLA-B homozygotes in the donor, so that cells from these donors could cover a high number of patient populations, and the rejection response caused by inconsistencies in HLA-B subtypes was reduced. HLA-B, such as B*40 homozygote, B*15 homozygote, B*46 homozygote, B*13 homozygote, B*51 homozygote, B*58 homozygote, B*07 homozygote, B*35 homozygote, B*44 homozygote, B*52 homozygote, B*57 homozygote, B*54 homozygote, and B*55 homozygote, was mainly selected, which had relatively high frequency in the population. HLA-A, such as A*02 homozygote, A*11 homozygote, and A*02/A11 heterozygote, was selected, which had relatively high frequency in the population.

### 2.3 Preparation of CD3⁺ T Cells

### (1) Isolation of PBMCs from Peripheral Blood

Peripheral blood was collected from healthy donors and diluted with PBS buffer at a rate of 1:1. A cell isolation solution (Ficoll) with a blood volume of ⅓ after dilution was first added into a new 50 mL centrifuge tube, then the blood cell dilution was added very slowly along the tube wall, and the mixture was centrifuged at room temperature for 20 min at 800 g (for the centrifuge, the acceleration speed was set as 1, and the deceleration speed was set as 0). After centrifugation, the liquid in the centrifuge tube was divided into PBS, a serum layer, a leucocyte layer, a lymphocyte isolation solution, and a red blood cell layer from top to bottom. The PBS and serum layer were removed. The leucocyte layer was transferred to a new 50 mL centrifuge tube, PBS was added to 40 mL to wash the cells, and the mixture was centrifuged for 10 min at 450 g. The supernatant was discarded after centrifugation to obtain peripheral blood mononuclear cells. The cells were resuspended and then subjected to cell counting.

### (2) Thawing of Cryopreserved Healthy Human PBMCs

Cryopreserved healthy human PBMC cells were thawed in a 37° C. water bath. After complete thawing, the cells were pipetted into a 15 mL centrifuge tube containing 10 mL of X-VIVO15 culture medium containing 10% FBS (purchased from LONZA), and centrifuged for 8 min at 400 g; the supernatant was discarded, 2 mL of X-VIVO15 culture medium (containing 10% FBS and DNase I with a final concentration of 100 μg/mL) was added, and the cells were incubated at room temperature for 15 min and shaken continuously during incubation; the solution after incubation was filtered by using a 40 μm filter; 10 mL of PBS buffer was pipetted to resuspend the cells at the bottom, and then the cells were added onto the filter; the cells were centrifuged for 8 min at 400 g after filtration, and the supernatant was discarded after centrifugation; the cells were resuspended and then subjected to cell counting.

### (3) Sorting of CD3⁺ T Cells

T cells in the peripheral blood mononuclear cells (PBMCs) were extracted using a EasySep™ human T cell sorting kit (purchased from StemCell Technologies, Catalog No. 17951). The density of PBMCs was adjusted to 5×10⁷ cells/mL, and a PBS buffer was added in a range of 0.25-2 mL; a cocktail was added firstly and mixed uniformly, and an isolation cocktail was then added at 50 μL/mL; after uniform mixing, the mixture was left at room temperature for 5 min; the RapidSpheres were vortexed by a vortex oscillator for 30 s, added into the cells at 40 μL/mL, and mixed uniformly; the mixture was supplemented with a buffer to the fold of 2.5 mL, and gently pipetted up and down for 2-3 times; the mixture was added into cryopreservation tubes with 2.5 mL in each tube, and the tubes were placed on a magnetic frame and left at room temperature for 3 min; the covers of the cryopreservation tubes were gently removed, and the magnetic frame was carefully picked up by holding two ends of the magnetic frame and inverted for 2-3 s; the cell liquids were pouring into new centrifuge tubes at one time; the cells were resuspended in 10-20 mL of a buffer (depending on the number of cells) and then centrifuged for 10 min at 300 g, and the supernatant was discarded to obtain CD3⁺ T cells.

### (4) Activation of T Cells

An activating reagent was prepared according to a volume ratio of culture medium: Transact=99:1, the culture medium was X-VIVO15 culture medium (containing 5% FBS, 200 U/mL IL2, 10 ng/mL IL7, and 5 ng/ml IL15), and Transact was purchased from Miltenyi. The T cells were thoroughly resuspended in 1 mL of activating reagent (containing 10 μL of Transact) per 1×10⁶ cells, and then incubated in an incubator with 5% CO₂ at 37° C. for 1 day.

### Example 3

### 3.1 Virus Transfer

CD3⁺ T cells were obtained according to the method in Example 2 (D0) and activated with CD3/CD28 antibody magnetic beads. After activation, lentiviral vectors (B7H3 CAR lentiviral expression vectors prepared in Example 1) were transfected on D1, the lentiviral vectors were washed off on D2, and electroporation was performed on D3.

### 3.2 Gene Knockout

RNP complexes were transferred to the activated T cells prepared in Example 3.1 (the CAR-T cells on D3 were used as starting cells) by electroporation using an electroporation kit (purchased from LONZA, Catalog No. V4XXP-3024). After sampling and counting, the cells were collected and centrifuged, and the cell pellet was resuspended in PBS. A culture medium (X-VIVO15 culture medium+10% FBS+IL2 (200 U/mL)+IL7 (10 ng/mL)+IL15 (5 ng/ml)) was pre-heated for 30 min in advance in a well plate. An electroporation buffer was prepared according to a ratio of Nucleofector Solution:Supplement=82:18; the RNP complexes were distributed according to each electroporation system using 1×10⁷ cells (Cas9:sgRNA=2:1). 10 μg of sgRNA was first added to a PCR tube (without RNase), 20 μg of Cas9 protein (purchased from Thermo, Catalog No. A36499) was then added, and the mixture was mixed gently and incubated at room temperature for 12 min. The cells described above were counted and centrifuged for 8 min at 300 g, and the supernatant was discarded. PBS was added to resuspend the cells, 1E7 cells were pipetted and centrifuged again for 8 min at 300 g, and the supernatant was discarded. The cells were resuspended in 100 μL of the prepared electroporation buffer. The incubated RNP complexes were added to the cell suspension described above. The mixture was gently mixed and gently transferred to an electroporation cuvette. The electroporation cuvette was placed on a Lonza-4D electroporation apparatus and subjected to electroporation using an EO-115 electroporation program. A pre-heated culture medium was added into the electroporation cuvette, the cells were transferred into the pre-heated culture medium in the well plate by using a matched pipette and then placed in an incubator with 5% CO₂ at 37° C. for 48 h, and then the cells were collected. The editing efficiency was assayed by Sanger sequencing, and the knockout efficiency of the collected cells was assayed by FACS.

sgRNA sequences were as follows: TRAC sgRNA: AGAGTCTCTCAGCTGGTACA (SEQ ID NO: 157), A02 sgRNA: CTGACCATGAAGCCACCCTG (SEQ ID NO: 174), and A11 sgRNA: GGCCCCTCCTGCTCTATCCA (SEQ ID NO: 204).

### 3.3 Sorting of CD3-Negative T Cells

CD3-negative T cells were sorted. The cells were counted and centrifuged, and the supernatant was discarded; the cells were resuspended in a buffer and mixed uniformly; CD3 magnetic beads were added according to 20 μL of CD3 magnetic beads per 10⁷ cells, and the mixture was mixed uniformly and incubated in a refrigerator at 4° C.; the cells were washed with the buffer and centrifuged, and then the magnetic beads were isolated; a column was first put on a magnetic pole, and a centrifuge tube was correspondingly put below the magnetic pole; the column (LD) was infiltrated in the buffer, and the cells were added onto the column without generating bubbles; the column was washed 2 times with the buffer, the washed liquid (CD3-T) was collected in a 15 mL centrifuge tube, and a part of the cells were subjected to cell counting.

### 3.4 Cell Culturing

The cell state was observed under a microscope. The cells were diluted, counted, and supplemented with a full culture medium to maintain the cell density at 3×10⁵ to 1×10⁶ cells/mL. Liquid was supplemented/changed in the middle time, and the cells were cultured at 37° C. with 5% CO₂. Cell harvesting: the cell suspension was collected in a cell centrifuge tube and centrifuged, and the supernatant was discarded. The cells were washed with normal saline again and centrifuged. A cryopreservation solution was prepared, and the centrifuged cells were resuspended in the cryopreservation solution. The cell suspension was pipetted to a cell cryopreservation bag for a final product by using a syringe, and the cell cryopreservation bag was labeled for the later cryopreservation.

### 3.5 Assay of Gene Knockout Efficiency

### (1) Sanger Sequencing Assay

The cells were counted. 3×10⁴ to 5×10⁴ cells were centrifuged for 5 min at 2000 r/min, and the supernatant was discarded as much as possible. 20 μL of DE lysis buffer was added into each tube. The lysed cells were added into a PCR tube, centrifuged instantaneously, and then placed into a PCR apparatus with the following conditions: 65° C. for 30 min, 4° C. for 30 s, 95° C. for 2 min, and 16° C. for infinite time. PCR was performed by using primer pairs TRAC-For/TRAC-Rev or HLA-A For/HLA-A Rev, and the lysed product was used as a template. The PCR product was sent to Genewiz for Sanger sequencing. After obtaining the Sanger sequencing results, the editing site and the editing efficiency were predicted with the EditR editor on the website: https://moriaritylab.shinyapps.io/editr_v10/.

### (2) Cell Counting by Flow Cytometry

10E5 to 10E8 cells were centrifuged for 5 min at 2000 rpm, and the supernatant was discarded. 100 μL of PBS buffer was added to each tube to resuspend the cells, and 5 μL of anti-human AB TCR-APC antibody (purchased from eBioscience), 5 μL of HLA-A02 monoclonal antibody (BB7.2), APC, and eBioscince™ antibody (purchased from Invitrogen) were added. The mixture was mixed uniformly and incubated at room temperature for 10 min. After being centrifuged for 5 min at 2000 rpm, the cells were washed 2 times with the PBS buffer, resuspended, and assayed by a BD FACSAria flow cytometer. The positive expression rates of TCR and HLA-A02 on the cell surface could be obtained. Knockout efficiency=(A−B)/A×100%, wherein A was the positive expression rate of the control group; B was the positive expression rate of the knockout group.

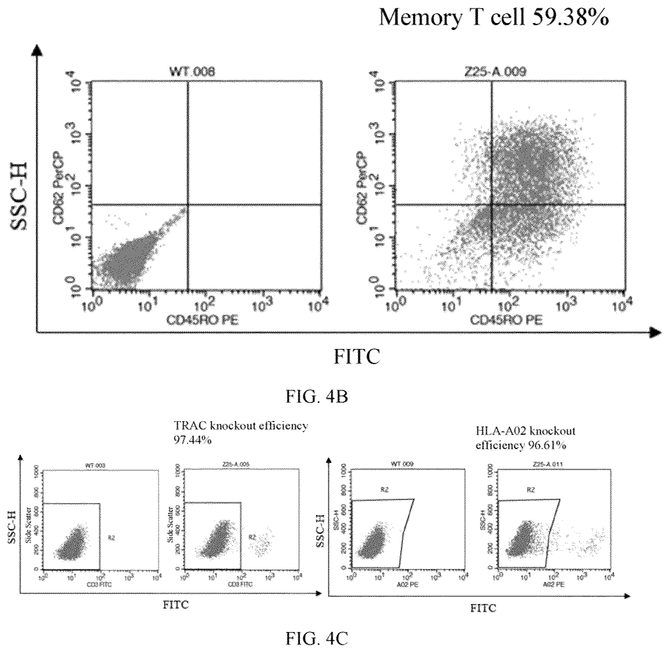

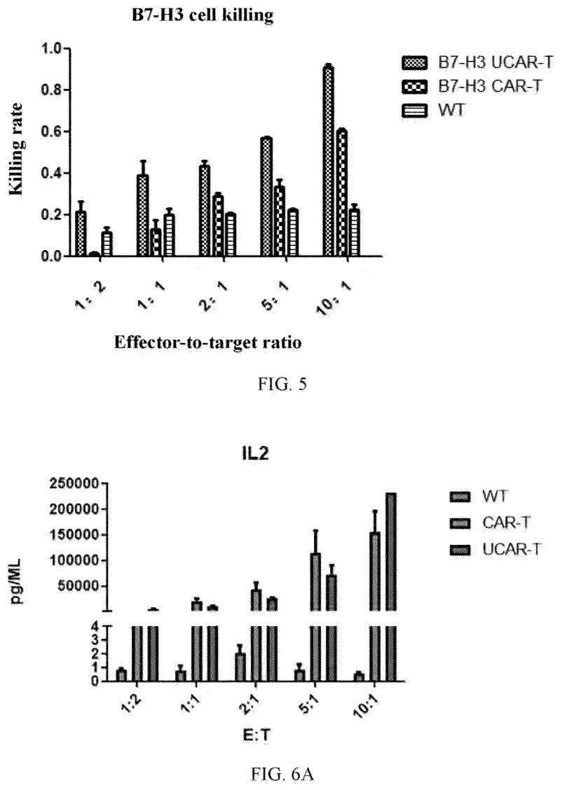

The results are shown in FIGS. 5A-5C. The CAR positive rate of anti-B7H3 UCAR-T cells can be more than 30% (FIG. 4A), the central memory ratio of the anti-B7H3 UCAR-T cells is about 50% (FIG. 4B), and the double knockout efficiency of the anti-B7H3 UCAR-T cells is up to 90% (FIG. 4C).

### Example 4. In Vitro Cytotoxicity Analysis of Anti-B7H3 UCAR-T Cells

### 4.1 Killing of Anti-B7H3 UCAR-T Cells for Target Cells

(1) B7H3 target cells: PANC-1-Luciferase; the state of the target cells was adjusted to the log phase, and the cells were continuously passaged 2 times before experiments;
(2) The anti-B7H3 UCAR-T cells and T cells in a control group anti-B7H3 CAR-T were prepared. The knockout efficiency, transfection efficiency, CD3-T sorting efficiency, and the proportion of memory T cells were assayed by flow cytometry, and the amplification fold was counted;
(3) several groups of prepared cells were collected by centrifugation, each group of 6×10⁶ cells;
(4) the target cells were resuspended in 1640+10% FBS. For each target, three 24-well plates were taken, and the target cells were seeded at 2×10⁵ cells/well (both target and effector cells were seeded at a density of 2×10⁶ cells/mL). Effector cells were then added in an E/T (effector-to-target ratio, effector cell:target cell) ratio. Each well was supplemented to a maximum volume (e.g., 600 μL). The same amount of target cells were seeded in the control group, without effector cells (600 μL). The well plates were incubated in an incubator with 5% CO₂ at 37° C. for 24 h. The cells were plated in the following E/T: 1:2, 1:1, 2:1, 5:1, and 10:1 and repeated three times; and
(5) After 24 h of culture, the well plates were taken out of the incubator, and 200 μL of supernatant was collected. The lysis capacity of the recombinant CAR-T cells on the target cells was then reflected by the assay of Luciferase activity.

The calculation formula for the lysis percentage of the target cells was as follows:

$$Lysis \% = ( 1 - \frac{Luc {Activity}_{Mixed sample}}{Luc {Activity}_{Control sample}} ) \times 100 \%$$

Analysis of results: the anti-B7H3 CAR-T cells and anti-B7H3 UCAR-T have a significant killing effect on the PANC-1-Luciferase cells. The anti-B7H3 UCAR-T cells can achieve more than 90% of killing efficiency when the effector-to-target ratio is 10:1 (see FIG. 5).

### 4.2 Cytokine Secretion Assay of Anti-B7H3 UCAR-T Cells Co-Cultured with Target Cells

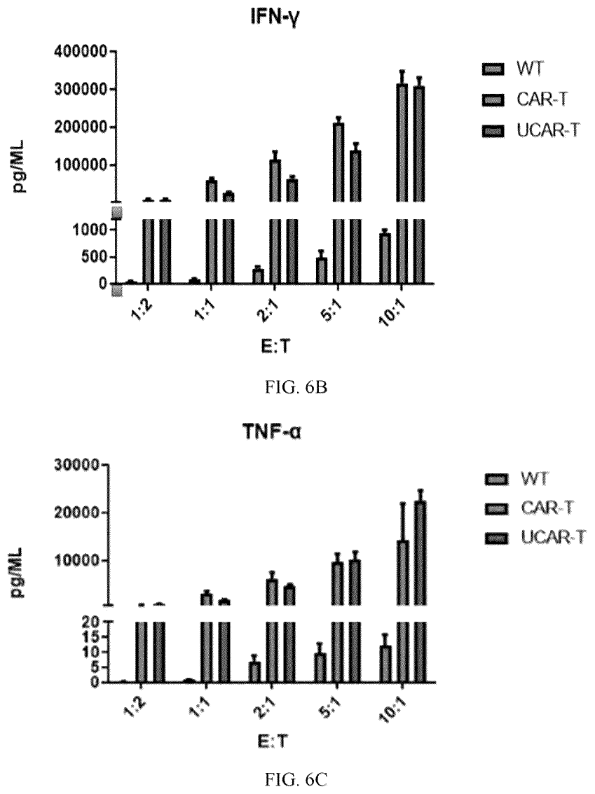

The supernatant of the co-culture system described above was collected, and the cytokine secretion level was assayed. Analysis of results (FIGS. 6A-6C): anti-B7H3 CAR-T and anti-B7H3 UCAR-T can be significantly activated and secrete IL-2, IFN-γ, and TNF-α cytokines in large quantities.

### Example 5. In Vivo Anti-Tumor Effect of Anti-B7H3 UCAR-T Cells

NSG mice aged 8-10 weeks were injected subcutaneously with tumor cells PANC-1-Luciferase-GFP (5×10⁶) and divided into three groups of 5 mice per group, and the tumor formation time was generally 2-4 weeks. 5E6 anti-B7H3 UCAR-T cells, B7H3 CAR-T cells, and T cells without gene knockout were separately injected intratumorally into each group of mice by single-point injection with an injection volume of 50 μL. Tumor regression in mice was monitored by luciferase.

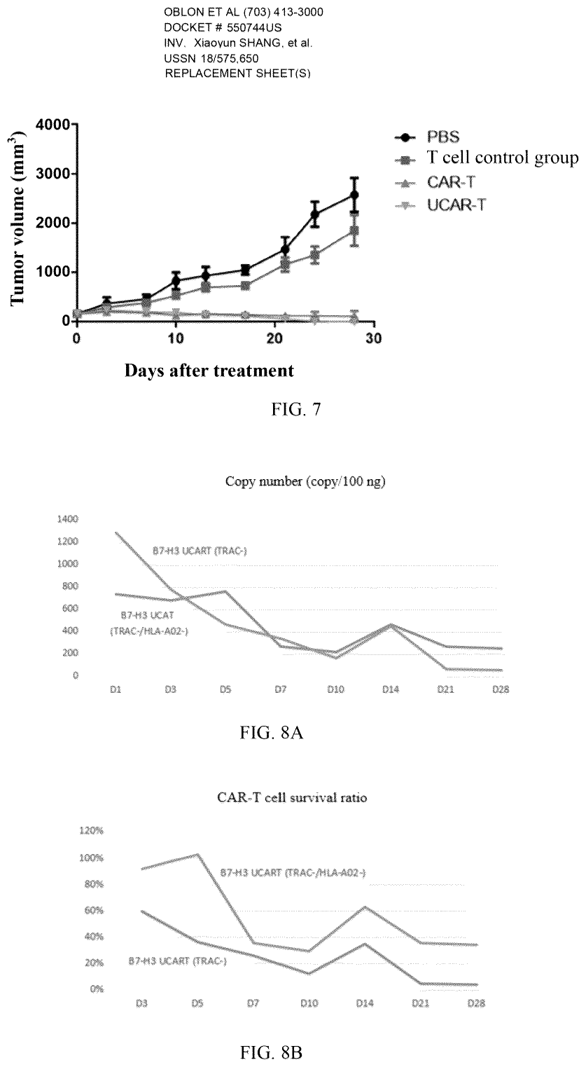

Analysis of results (FIG. 7): the tumor growth rate of the mice reinfused with the anti-B7H3 UCAR-T cells was significantly slowed down, and both anti-B7H3 CAR-T and anti-B7H3 UCAR-T show excellent anti-tumor effects.

### Example 6. In Vivo Half-Life Assay of Anti-B7H3 UCAR-T Cells

15 humanized immune system mice (hHSC-NCG) were prepared and divided into 3 groups. Cells were prepared as follows: an experimental group: anti-B7H3 UCAR-T cells (TRAC+HLA-A02 knocked out); control group 1: B7H3 CAR-T; and control group 2: anti-B7H3 UCAR-T cells (TRAC+B2M knocked out). Each mouse was injected with 1×10⁷ cells, and blood was collected at different time points: D0, 2 h, D3, D7, D14, D21, D28, D35, D42, D49, D56, and D60. Genomes in blood samples at different time points were extracted, and copy/ng genome DNA was calculated by QPCR absolute quantification method. UCAR-T cells harvested on day 14 were used as a positive control, and DEPC water was used as a negative control.

Analysis of results: the anti-B7H3 UCAR-T cells (TRAC+HLA-A02 knocked out) survive in mice for the longest time.

### Example 7. In Vitro Safety Validation of Universal T Cells

(1) GVHD response: T cells with double knockout of TRAC and HLA-A and T cells without gene knockout were prepared, allogeneic PBMCs were irradiated, 2 groups of prepared cells were stimulated separately, and IFN-γ levels were assayed.

Analysis of results: the T cell group with double knockout of TRAC and HLA-A had very low IFN-γ secretion level, indicating that the knockout of TRAC reduces the GVHD response.

(2) Allogeneic response: after the allogeneic PBMCs were stimulated and irradiated, 2 groups of cells were assayed for the IFN-γ level.

Analysis of results: the T cell group with double knockout of TRAC and HLA-A had very low IFN-γ secretion level, indicating that the knockout of HLA-A reduces the allogeneic response.

### Example 8. In Vivo Safety Validation of Universal T Cells

Experimental group: 5×10⁶ TCR-HLA-A-double negative anti-B7H3 UCAR-T cells and 5×10⁶ allogeneic T cells were co-injected into NSG mice.

Control group: 5×10⁶ TCR-B7H3 UCAR-T cells and 5×10⁶ allogeneic T cells were injected into NSG mice.

Each group contained 5 NSG mice.

(1) GVHD response: according to clinical criteria, such as survival rate, coat texture, skin integrity, and the like, graft versus host response was observed. Assay of cytokines: peripheral blood serum was collected to assay the levels of cytokines such as IL6, IL-2, TNF-α, IFN-γ, and the like. The blood collection time points were as follows: 24 h, D3, D7, D14, D28, and 2 M before reinfusion. Assay of visceral lesion: at the end of the observation period (about 2 months), spleen, liver, skin, gastrointestinal tract, lung, and kidney of the mice were collected for HE section staining analysis.

Analysis of results: of 5 mice injected with untreated T cells, 4 mice developed lethal xenograft versus host disease (GVHD) within 2 months after injection. None of the mice receiving cells with double knockout of TRAC and HLA-A developed GVHD; the T cell group with double knockout of TRAC and HLA-A had very low secretion levels of cytokines such as IL6, IL-2, TNF-α, and IFN-γ; moreover, different organs in the mice were morphologically normal, indicating that the GVHD response in the T cell group with double knockout of TRAC and HLA-A is greatly reduced.

(2) Allogeneic response: CAR-T cells with double knockout of TRAC and HLA-A were prepared, and 1×10⁷ TCR-HLA-A-double-knockout CAR-T cells and 2×10⁶ allogeneic T cells were co-injected into NSG mice. Control group: 1×10⁷ TCR⁻CAR-T cells were injected into NSG mice.

Blood was collected at different time points to determine CAR copy number. The changes in the copy number were compared for both groups of CARs. The time points were as follows: D1, D5, D7, D10, D14, D21, and D28.

Conclusion: on D21, the rejection response of the mice in the control group was significant, and the copy number was basically undetectable. However, the copy number of the experimental group was still at a relatively stable level, indicating that the rejection response is significantly weakened; the survival time of the cells in the experimental group was prolonged in the mice, indicating that the rejection response in the CAR-T cell group with double knockout of TRAC and HLA-A is greatly reduced (see FIGS. 8A-8B).

### Example 9. Safety Analysis of Gene Editing

T cells with double knockout of TRAC and HLA-A and T cells without gene knockout were prepared, and after the assay of knockout efficiency, the following analyses were performed:

(1) Off-target:

Control group: transgenic CAS9+ODN tag

Experimental group: transgenic CAS9+sgRNA (TRAC+HLA-A)+ODN tag

On-target and off-target-WGS (whole genome sequencing): on D14, 1×10⁶ of T cells without gene knockout and T cells with double knockout of TRAC and HLA-A were each collected and sent to Suzhou Genewiz Biological Technology Co., Ltd.

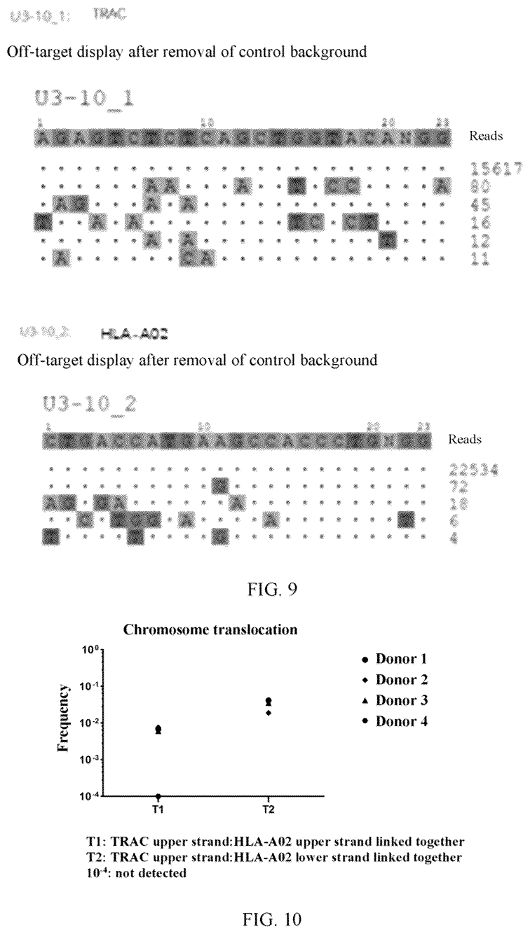

Analysis of results: the off-target rate of the experimental group was very low, and the off-target was mainly concentrated among genes and on introns, so that the effect of the off-target on gene functions is not great (see FIG. 9).

(2) Chromosomal translocation: the qPCR method was used to quantify rearrangements that may occur when editing both TRAC and HLA loci simultaneously. The two translocations were labeled as TRAC:HLA and HLA:TRAC. Positive reference samples in the synthesized template plasmid were evaluated as assay controls. Amplified fragments on both sides of the target region of the HLA genome were used as internal controls. The genome DNA was extracted to perform real-time quantitative PCR, and the gene copy number of the genome DNA was calculated according to the standard curve and Cq value.

Analysis of results: the T cells with double knockout (TRAC+HLA-A) were assayed for the occurrence of chromosomal translocation on D14 (harvest), and the assay results showed that the assay values for two types of translocation methods were closed to zero, suggesting that there is no rearrangement of the locus (see FIG. 10).

(3) Karyotyping: 1×10⁶ ofT cells without gene knockout and T cells with double knockout of TRAC and HLA-A, which had a confluence of 70%-80%, were each put into two T25 bottles. The bottles were filled with a culture medium, covered with a fully sealed lid, wrapped with a sealing film, and sent to Zhejiang Ruyao Biotech Co., Ltd. for assay.

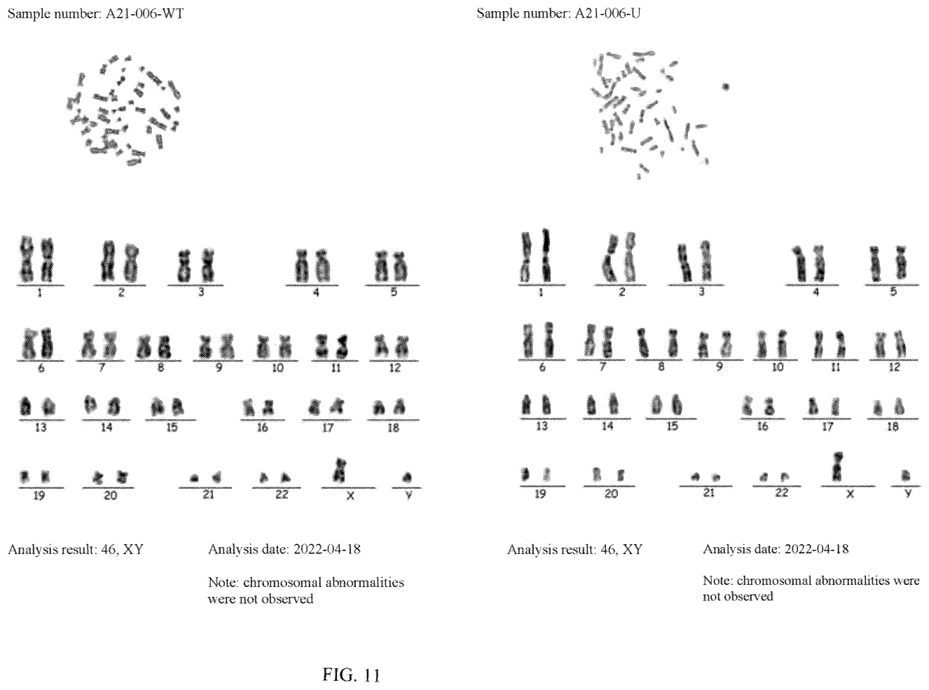

Analysis of results: compared with the control group, the experimental group was normal in the karyotype (see FIG. 11).

(4) Cas9 protein residue: when the cells were prepared, 1×10⁶ cells at three time points, before knockout, after knockout, and before harvest, were separately collected for lysis, then a protein quantification kit (NOVATEINBIO, Catalog No. NB-E1372PR) was used for quantification, and each group of samples were adjusted to be 2 μg of the same sample loading amount and were assayed by a CRISPR/Cas9 protein ELISA kit according to the instruction. The Cas9 protein in the sample was firmly and stably attached to a test paper hole. The bound Cas9 protein was then recognized using an assay antibody and then developed with a developing agent. The Cas9 ratio was directly proportional to the absorbance, and absolute amounts of Cas9 protein were quantified by comparison to Cas9 control samples.

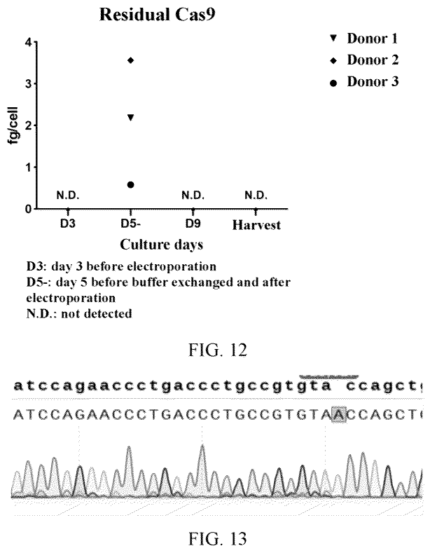

Analysis of results: the T cells with double knockout (TRAC+HLA-A) were assayed for the residue of spCas9 at four time points, before electroporation (D3), before buffer exchange and after electroporation (D5), D9, and D14 (harvest). The residues were not detected at all three time points except for the assay of trace residue before buffer exchange and after electroporation (D5) (see FIG. 12).

### Example 10. Preparation of T Cells with Single-Gene Knockout

RNP complexes were transferred to the activated T cells prepared in Example 2 by electroporation using an electroporation kit (purchased from LONZA, Catalog No. V4XXP-3024). A culture medium (X-VIVO15 culture medium+10% FBS+IL2 (200 U/mL)+IL7 (10 ng/mL)+IL15 (5 ng/mL)) was pre-heated for 30 min in advance in a well plate. An electroporation buffer was prepared according to a ratio of Nucleofector Solution:Supplement=82:18. Preparation of RNP complexes: the sgRNA sequence of TRAC was Sg9 (set forth in SEQ ID NO: 157), and the sgRNA sequence of HLA-A was HLA-A02 Sg2 (set forth in SEQ ID NO: 173), HLA-A02 Sg5 (set forth in SEQ ID NO: 174), HLA-A11 Sg21 (set forth in SEQ ID NO: 204), or HLA-A11 Rsg2 (set forth in SEQ ID NO: 203). 20 μg of sgRNA was first added into a PCR tube (without RNase), 10 μg of Cas9 protein (purchased from Thermo, Catalog No. A36499) was then added, and after being mixed gently, the mixture was incubated at room temperature for 12 min. The activated T cells cultured in Example 2 were counted and centrifuged for 8 min at 300 g, and the supernatant was discarded. PBS was added to resuspend the cells, 1E7 cells were pipetted and centrifuged again for 8 min at 300 g, and the supernatant was discarded. The cells were resuspended in 100 μL of the prepared electroporation buffer. The incubated RNP complexes were added to the cell suspension described above. The mixture was gently mixed and gently transferred to an electroporation cuvette. The electroporation cuvette was placed on a Lonza-4D electroporation apparatus and subjected to electroporation using an EO-115 electroporation program. A pre-heated culture medium was added into the electroporation cuvette, and the cells were transferred into the pre-heated culture medium in the well plate by using a matched pipette and then placed in an incubator with 5% CO₂ at 37° C.

### Example 11. Comparison of Gene Knockout Efficiency Assay Methods

### (1) Sanger Sequencing Assay

The cells were counted. 3×10⁴ to 5×10⁴ cells were centrifuged for 5 min at 2000 r/min, and the supernatant was discarded as much as possible. 20 μL of DE lysis buffer was added into each tube. The lysed cells were added into a PCR tube, centrifuged instantaneously, and then placed into a PCR apparatus with the following conditions: 65° C. for 30 min, 4° C. for 30 s, 95° C. for 2 min, and 16° C. for infinite time. PCR was performed by using primer pairs TRAC-For/TRAC-Rev or HLA-A For/HLA-A Rev, and the lysed product was used as a template. The PCR product was sent to Genewiz for Sanger sequencing. After obtaining the Sanger sequencing results, the editing site and the editing efficiency were predicted with the EditR editor on the website: https://moriaritylab.shinyapps.io/editr_v10/.

### (2) TA Cloning Sequencing Assay

The PCR product was purified using AxyPrep™ PCR product cleaning kit (purchased from AXYGEN), and then a sticky end was added to the purified PCR product using a kit (DNA A-Tailing Kit, purchased from TaKaRa). The product was ligated to T vector (pMDTM19-T Vector Cloning Kit, purchased from TaKaRa) by a DNA Ligation Kit Ver2.1 (purchased from TaKaRa), and the ligated product was transformed into competent cells (DH5 alpha). The cells were coated on an LB plate containing ampicillin resistance, and the plate was incubated in an incubator at 37° C. for about 12 h. Subsequently, a single colony was picked, and the single colony was sent to Genewiz for sequencing. Knockout efficiency=number of mutated clones/total clones.

### (3) Cell Counting by Flow Cytometry

10E5 to 10E8 cells were centrifuged for 5 min at 2000 rpm, and the supernatant was discarded. 100 μL of PBS buffer was added to each tube to resuspend the cells, and 5 μL of anti-human AB TCR-APC antibody (purchased from eBioscience), 5 μL of HLA-A02 monoclonal antibody (BB7.2), APC, and eBioscince™ antibody (purchased from Invitrogen) were added. The mixture was mixed uniformly and incubated at room temperature for 10 min. After being centrifuged for 5 min at 2000 rpm, the cells were washed 2 times with the PBS buffer, resuspended, and assayed by a BD FACSAria flow cytometer. The positive expression rates of TCR and HLA-A02 on the cell surface could be obtained. Knockout efficiency=(A−B)/A×100%, wherein A was the positive expression rate of the control group; B was the positive expression rate of the knockout group.

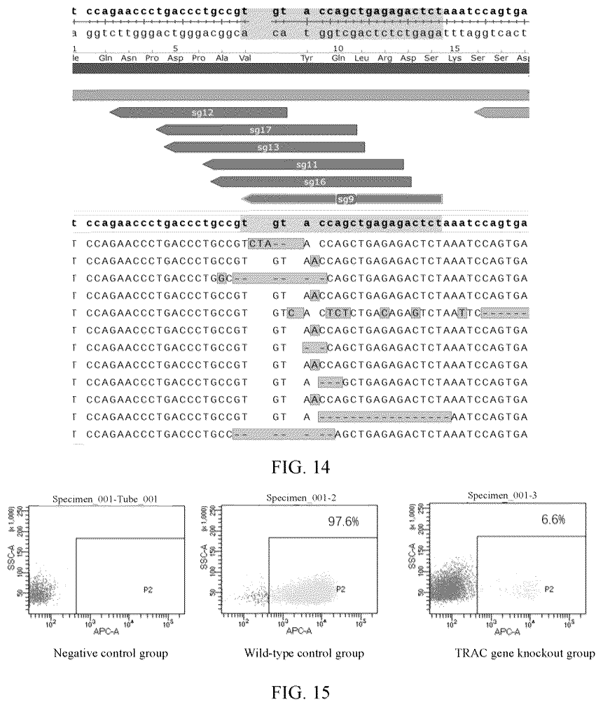

Three assay results of TRAC single-gene knockout are shown in FIGS. 13 to 15, and calculation results of knockout efficiency are shown in Table 2. The three assay methods were basically the same, and the editing efficiency was assayed only by Sanger sequencing in subsequent experiments.

TABLE 2
Assay method results of gene knockout efficiency

| Target gene | sgRNA | Assay method | Knockout efficiency % |
|---|---|---|---|
| TRAC | Sg9 | Sanger sequencing | 90 |
| | | TA cloning sequencing | 95 |
| | | Flow cytometry | 93 |

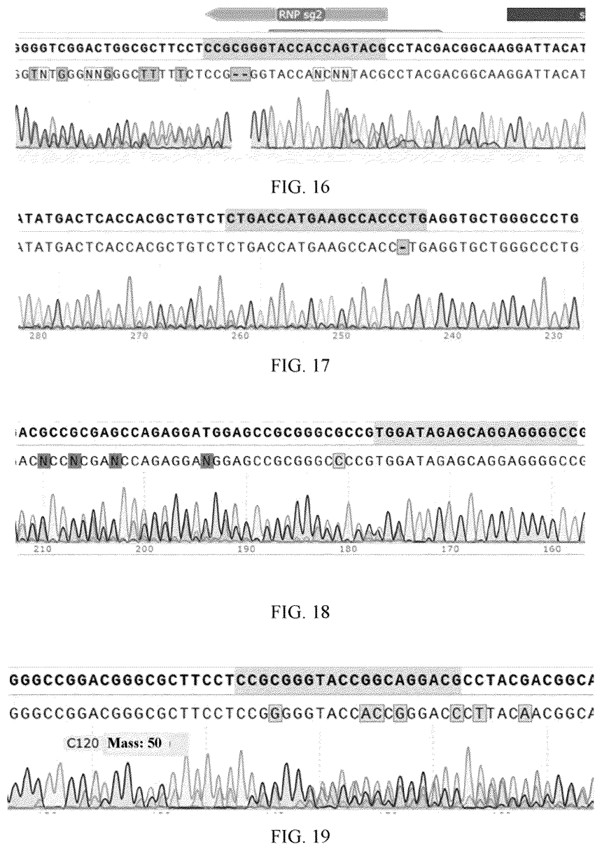

The results of the Sanger sequencing method for HLA-A02 gene editing are shown in FIGS. 16-17, and the editing efficiencies are both 90%; the results of the Sanger sequencing method for HLA-All gene editing are shown in FIGS. 18-19.

### Example 12. Preparation of T Cells with Double-Gene Knockout of TRAC Gene and HLA-A Gene

RNP complexes were transferred to the activated T cells prepared in Example 2 by electroporation using an electroporation kit (purchased from LONZA, Catalog No. V4XXP-3024). A culture medium (X-VIVO15 culture medium+10% FBS+IL2 (200 U/mL)+IL7 (10 ng/mL)+IL15 (5 ng/mL)) was pre-heated for 30 min in advance in a well plate. An electroporation buffer was prepared according to a ratio of Nucleofector Solution: Supplement=82:18. Preparation of RNP complexes: 20 μg of TRAC sgRNA (TRAC Sg9) and 20 μg of HLA-A sgRNA (HLA-A02 Sg2, HLA-A02 Sg5, HLA-A11 sg21, or sgRNAs targeting HLA-A*24:02:01, HLA-A*30:01:01:01, HLA-A*33:01:01:01, HLA-A*03:01:01:01, HLA-A*01:01:01:01, or HLA-A*26:01:01:01) were separately added to PCR tubes (without RNA), 10 μg of Cas9 protein (purchased from Thermo, Catalog No. A36499) were added to each tube, and the tubes were mixed gently and incubated at room temperature for 12 min. The activated T cells cultured in Example 2 were counted and centrifuged for 8 min at 300 g, and the supernatant was discarded. PBS was added to resuspend the cells, 1E7 cells were pipetted and centrifuged again for 8 min at 300 g, and the supernatant was discarded. The cells were resuspended in 100 μL of the prepared electroporation buffer. The incubated RNP complexes of TRAC and HLA-A were added to the cell suspension described above. The mixture was gently mixed and gently transferred to an electroporation cuvette. The electroporation cuvette was placed on a Lonza-4D electroporation apparatus and subjected to electroporation using an EO-115 electroporation program. A pre-heated culture medium was added into the electroporation cuvette, and the cells were transferred into the pre-heated culture medium in the well plate by using a matched pipette and then placed in an incubator with 5% CO₂ at 37° C.

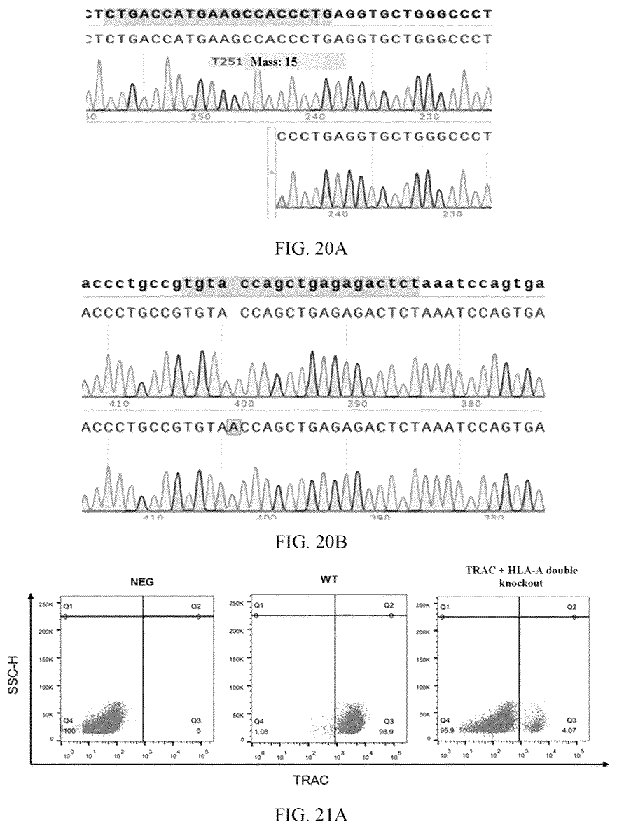

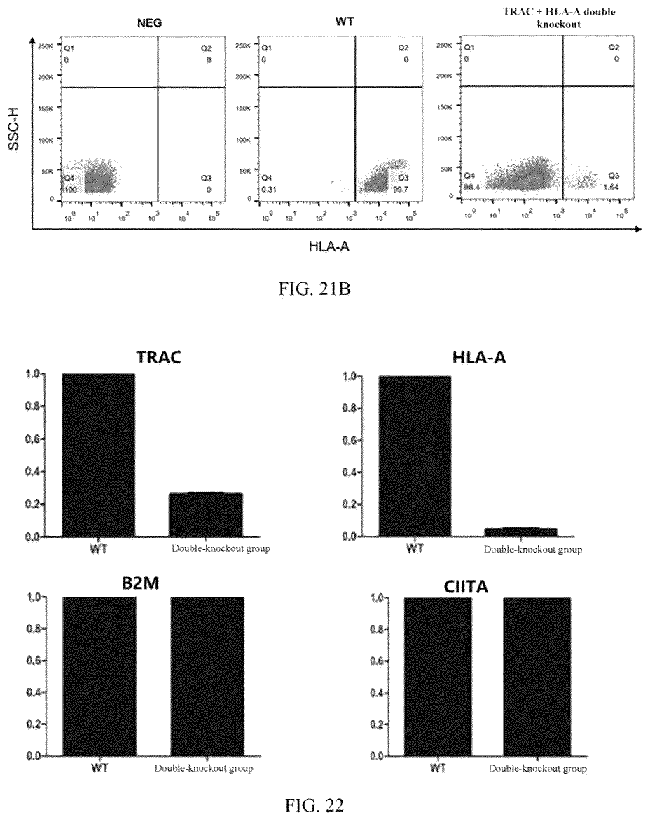

Double-gene knockout efficiency was assayed by sequencing, and TRAC-negative and HLA-A-negative T cells with double-gene knockout efficiency of not less than 80% could be obtained. The results are shown in FIGS. 20-21. FIG. 20A shows the results of HLA-A02 knockout using HLA-A02 Sg5, wherein the upper row shows the results of the control group (i.e., HLA-A02 Sg5 was not used for knockout); the next row shows the results of simultaneous knockout of HLA-A02 and TRAC. FIG. 20B shows the results of TRAC knockout using TRAC Sg9, wherein the upper row shows the results of the control group (i.e., TRAC Sg9 was not used for knockout); the next row shows the results of simultaneous knockout of HLA-A02 and TRAC. FIGS. 21A-21B show the knockout at protein levels after the knockout of HLA-A02 and TRAC, wherein NEG refers to a negative control, WT refers to the absence of any knockouts, and TRAC+HLA-A double knockout refers to the results of simultaneous knockout of HLA-A02 and TRAC.

### Example 13. Differences in Expression of TRAC, HLA-A, B2M, and CIITA Genes in T Cells with Double-Gene Knockout and Corresponding Genes in Corresponding Cells

(1) The activated T cells prepared in Example 2 were used and divided into two groups. One was used as a control, and the other was prepared into T cells with double-gene knockout of TRAC gene and HLA-A gene according to the method in Example 5. Sanger sequencing was performed according to the method in step (1) of Example 4. The cells with double-gene knockout of TRAC and HLA-A were obtained according to the sequencing result. The prepared T cells with double-gene knockout were incubated with corresponding TRAC and HLA-A antibodies, and a cell strain with double-gene knockout was obtained by flow cytometry sorting or magnetic bead sorting.

(2) A change in mRNA expression level in the T cells with double-gene knockout was assayed compared to the control group. RNA was extracted using an RNA extraction kit (purchased from QIAGEN, Catalog No. 74004), and reverse transcription was performed on RNA using a reverse transcription kit (purchased from Applied Biosystems, Catalog No. 4368814) to obtain cDNA. Quantitative PCR assay was performed using the cDNA as a template.

(3) A change in protein expression level in the T cells with double-gene knockout was assayed compared to the control group. Proteins were extracted using a whole protein extraction reagent (purchased from Thermo Scientific, Catalog No. 87787), and the protein expression level was assayed by Western Blot method or flow cytometry using TRAC antibody (purchased from eBioscience, Catalog No. 17-9986-42), HLA-A antibody (purchased from Merck, Catalog No. 17-9876-41), B2M antibody (purchased from Invitrogen, Catalog No. A15770), and CIITA antibody (purchased from OriGene, Catalog No. CF812200).

The Sanger sequencing assay found that the nucleotide sequence of TRAC and/or HLA-A genes in the T cells with double-gene knockout is changed relative to the control group; the quantitative PCR showed that the mRNA expression level of TRAC and/or HLA-A genes is down-regulated in the T cells with double-gene knockout, but the mRNA expression level of B2M and/or CIITA genes is not down-regulated. FACS and Western Blot results showed that the protein expression amount in the T cells with double-gene knockout is down-regulated, and the protein expression amount of B2M and/or CIITA is not down-regulated.

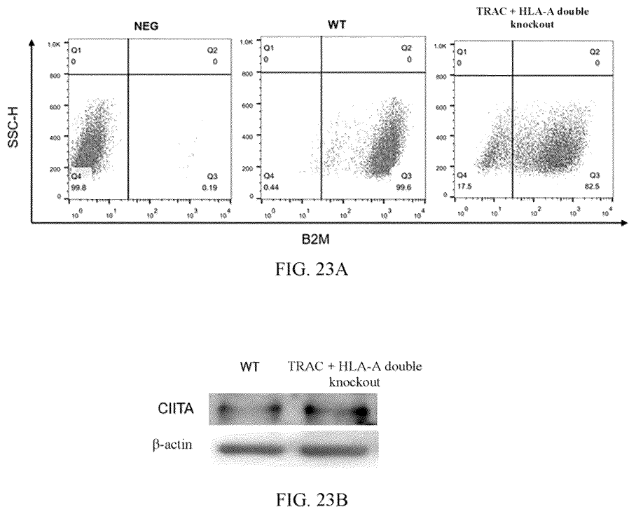

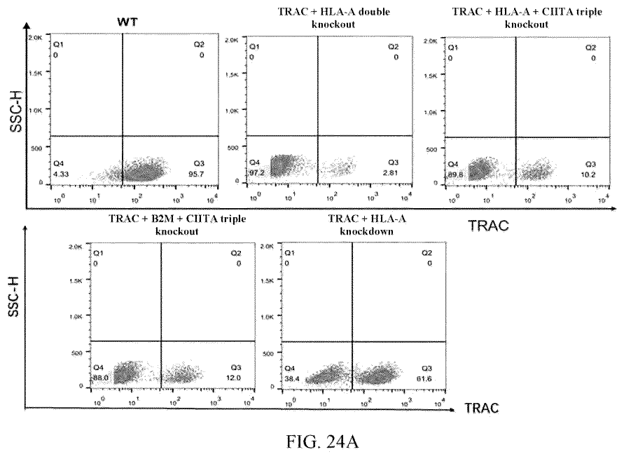

The results are shown in FIGS. 22-23. FIG. 22 shows mRNA level determination of gene expression, and FIG. 22 shows mRNA levels of TRAC, HLA-A, B2M, and CIITA, wherein WT refers to a case without any knockout treatment, and the double-knockout group refers to a result of T cells with double-gene knockout of the TRAC gene and the HLA-A gene. FIG. 23 shows protein level determination of gene expression, wherein FIGS. 23A-23B show protein expression levels of B2M and CIITA, respectively; NEG refers to a negative control, WT refers to a case without any knockout treatment, and TRAC+HLA-A double knockout refers to the result of T cells with double-gene knockout of the TRAC gene and the HLA-A gene.

### Example 14. Preparation of T Cells with Triple-Gene Knockout of TRAC Gene, HLA-A/B2M Gene, and CIITA Gene and Verification of Changes in Expression of Respective Three Genes

(1) A control group, cells with triple-gene knockout of TRAC gene, HLA-A gene, and CIITA gene, and cells with triple-gene knockout of TRAC gene, B2M gene, and CIITA gene were prepared according to the method in step (1) of Example 13.

(2) Changes in protein expression levels were assayed by FACS and Western Blot methods according to the method in step (3) of Example 13.

The protein expression levels of TRAC, HLA-A, and CIITA genes in the T cells with triple-gene knockout of TRAC, HLA-A, and CIITA were down-regulated relative to the cells in the control group; the protein expression levels of TRAC, HLA-A, and CIITA genes in the T cells with triple-gene knockout of TRAC, B2M, and CIITA were down-regulated relative to the cells in the control group.

(3) The knockout efficiencies of the cells with double-gene knockout in Example 13 and the two cells with triple-gene knockout in this example were assayed by flow cytometry using TRAC antibody (purchased from eBioscience, Catalog No. 17-9986-42), HLA-A antibody (purchased from Merck, Catalog No. 17-9876-41), and B2M antibody (purchased from Invitrogen, Catalog No. A15770), and the results showed that the efficiency of multiple gene knockout was achieved simultaneously at the single cell level, and that the efficiency of the double-gene knockout was significantly higher than that of the triple-gene knockout.

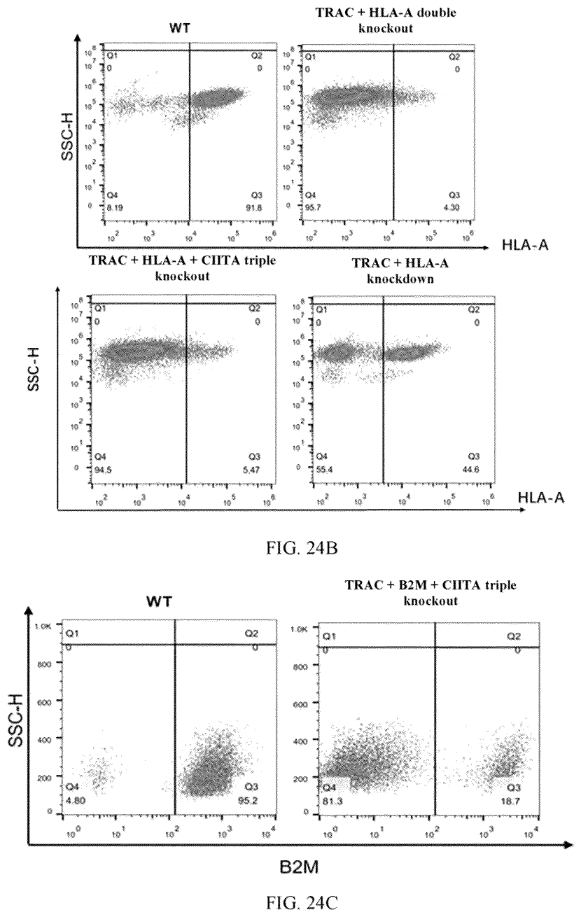

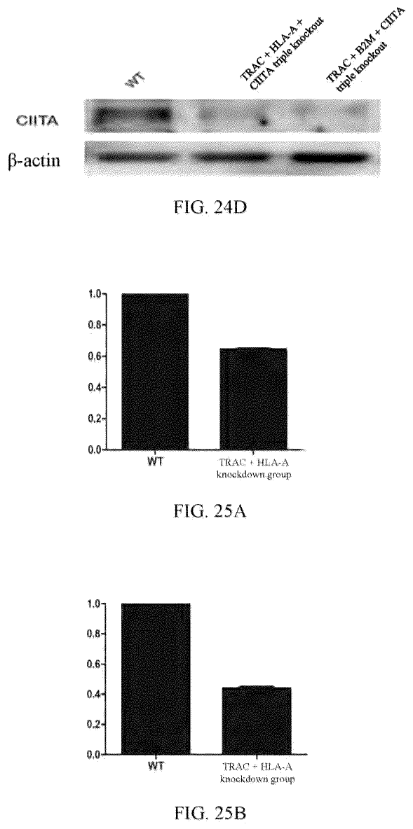

The results are shown in FIGS. 24A-24D. FIGS. 24A-24C show the knockout of TRAC, HLA-A and B2M at protein levels in sequence, wherein WT refers to a case without any knockout treatment, TRAC+HLA-A double knockout refers to the result of T cells with double-gene knockout of TRAC and HLA-A genes; TRAC+HLA-A+CIITA triple knockout refers to the result of T cells with triple-gene knockout of TRAC, HLA-A, and CIITA; TRAC+B2M+CIITA triple knockout refers to the result of T cells with triple-gene knockout of B2M, CIITA, and TRAC; and TRAC+HLA-A knockdown refers to the result of T cells with knockdown of TRAC and HLA-A genes prepared in Example 16. FIG. 24D shows the knockout of CIITA at the protein level.

The results in FIG. 24 showed that the protein levels of TRAC, HLA-A, CIITA, and B2M are down-regulated compared to the WT control group. Meanwhile, compared to TRAC+HLA-A+CIITA triple knockout or TRAC+B2M+CIITA triple knockout, the knockout efficiency of TRAC+HLA-A double knockout is higher.

### Example 15. Design of Antisense RNA Sequence

The transcription RNA sequences of the corresponding genes (TRAC gene and HLA-A gene) were obtained by the database https://www.ncbi.nlm.nih.gov/ or www.ensembl.org/, and siRNA was designed with reference to the following principles:

Sequences of 50-100 nucleotides downstream of the start codon and 100 nucleotides upstream of the stop codon were avoided as possible; sequences with less than 30 nucleotides in length were selected; 4 or more consecutive identical bases were avoided; intron regions were avoided; repetitive sequences were avoided; single nucleotide polymorphism (SNP) sites were avoided; sequences had a GC content ranging 30% to 60%; sequence patterns AA (N<sub>19), NA (N<sub>21), or NAR (N<sub>17) YNN were preferably selected, wherein A was adenosine, T was thymidine, R was adenosine or guanosine (purines), Y was thymidine or cytidine (pyrimidines), N was adenosine, thymidine, guanosine, or cytidine; homology comparison and analysis were performed on the selected sequences to avoid significant homology of the antisense RNA to other genes or sequences and the resulting off-target effects. The homology analysis was performed using NCBI Blast tool: Nucleotide-nucleotide BLAST (blastn), UCSC Blat tool, or Ensembl Blast.

The antisense RNA sequences obtained by design included HLA-A-homo-551, HLA-A-homo-NEG, TRAC-homo-375, and TRAC-homo-NEG.

### Example 16. Preparation of T Cells with Knockdown of TRAC Gene and HLA-A Gene

Double-gene knockdown was performed using the antisense RNA designed in Example 15. A lentivirus comprising the antisense RNA sequences of the TRAC gene and HLA-A gene was prepared by a company (Genepharma). CD3⁺ T cells were prepared according to the method in Example 2 (D0) and activated with CD3/CD28 antibody magnetic beads. The lentivirus carrying the antisense RNA sequences of the TRAC gene and HLA-A gene was transfected into the activated T cells (D1). On D2, the lentiviral vector was washed off, and the cells were continued to be cultured until D5. The T cells cultured until D5 were collected, and gene knockdown efficiency was assayed by quantitative PCR or Western Blot, etc. The obtained T cells were labeled with corresponding TRAC and HLA-A antibodies, and the T cells with knockdown of the TRAC gene and HLA-A gene could be obtained by flow cytometry sorting or magnetic bead sorting methods. The results showed that both the mRNA and protein expression levels of TRAC and HLA-A were down-regulated in the TRAC and HLA-A gene-knockdown group. FIGS. 25A-25B show the knockout of TRAC and HLA-A at mRNA levels in sequence, wherein WT refers to a case without any knockout treatment, and TRAC+HLA-A double knockout refers to the result of T cells with double-gene knockout of the TRAC gene and the HLA-A gene. Among these, the knockout level of TRAC and HLA-A at protein levels can be found in the results shown in FIG. 24.

### Example 17. Difference in Activities of Different T Cells

The T cells without gene knockout, with double-gene knockout, with triple-gene knockout, and with double-gene knockdown in Examples 2, 12, 14, and 16 were prepared, and several T cell activities were compared. Each group of cells was counted and seeded in 24-well plates with 1×10⁶ cells, and PHA (0.3 μg/mL) (ionomycin+) or 5 ng/mL PMA and 50 ng/mL ionomycin were added to the cells per well. The cells were cultured for another 5 h, and then the activation state of the cells were assayed using CD69 (early activated) (purchased from BD Biosciences, Catalog No. FN50) and CD137 (later stage) (purchased from BD Biosciences, Catalog No. 4B4-1) antibodies by flow cytometry. The results showed that the activities of the T cells with double-gene knockout and double-gene knockdown were superior to that of the T cells with triple-gene knockout.

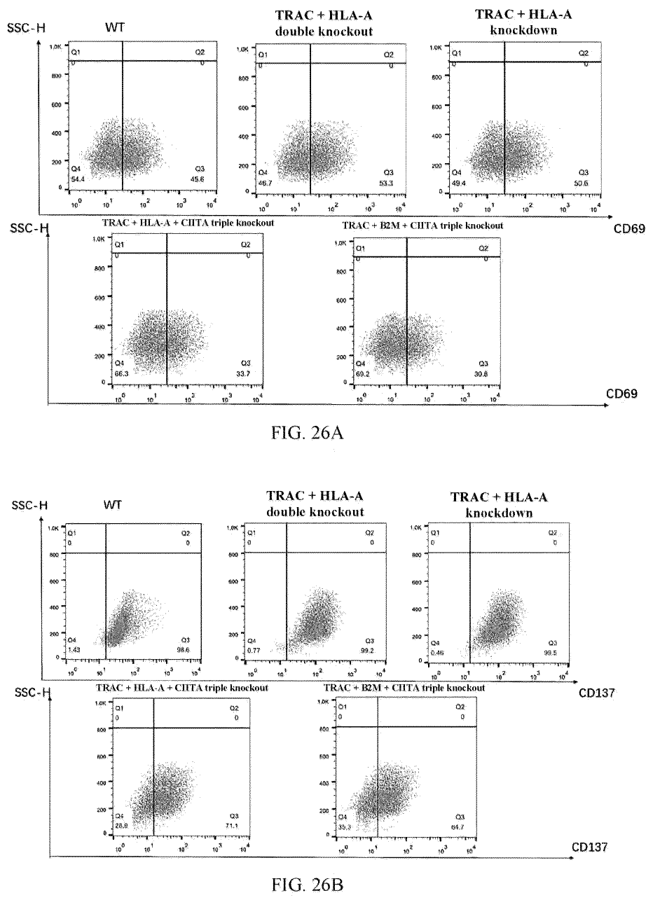

The expression of CD69 and CD137 at protein levels is shown in FIGS. 26A-26B, respectively, wherein WT refers to a case without any knockout treatment, TRAC+HLA-A double knockout refers to the result of T cells with double-gene knockout of TRAC and HLA-A genes; TRAC+HLA-A+CIITA triple knockout refers to the result of T cells with triple-gene knockout of TRAC, HLA-A, and CIITA; TRAC+B2M+CIITA triple knockout refers to the result of T cells with triple-gene knockout of B2M, CIITA, and TRAC; and TRAC+HLA-A knockdown refers to the result of T cells with knockdown of TRAC and HLA-A genes prepared in Example 16.

### Example 18. Difference in Reactivity of Different T Cells to Allogeneic NK Cells

CFSE (Invitrogen, C34554) labeling was performed on the T cells without gene knockout, with double-gene knockout, with triple-gene knockout, and with double-gene knockdown in Examples 2, 12, 14, and 16. The cells were counted, and 1×10⁶ cells were collected and co-cultured with NK cells (NK92MI) at a ratio of 1:1. After 24 h, the co-cultured cells were collected from each group, and the ratio of CFSE-positive cells in the mixed cells was determined by flow cytometry.

The results showed that the killing toxicity of the NK cells to the T cells with double-gene knockout and double-gene knockdown was lower than that of the T cells with triple-gene knockout. The results are shown in FIG. 27, wherein NK+T refers to a case where the NK cells were co-cultured with the T cells without any knockout treatment; NK+TRAC+HLA-A knockdown refers to a case where the NK cells were co-cultured with the resulting T cells with knockdown of the TRAC gene and HLA-A gene prepared in Example 16; NK+TRAC+HLA-A double knockout refers to a case where the NK cells were co-cultured with the T cells with double-gene knockout of the TRAC gene and HLA-A gene; NK+TRAC+HLA-A+CIITA triple knockout refers to a case where the NK cells were co-cultured with the T cells with triple-gene knockout of TRAC, HLA-A, and CIITA; NK+TRAC+B2M+CIITA triple knockout refers to a case where the NK cells were co-cultured with the T cells with triple-gene knockout of B2M, CIITA, and TRAC.

### Example 19. Difference in Allogeneic Immune Rejection of Different T Cells

Peripheral blood originated from donor I was used to prepare T cells without gene knockout, with double-gene knockout, with triple-gene knockout, and with double-gene knockdown in Examples 2, 12, 14, and 16. Peripheral blood originated from donor 2 was used to prepare CD3⁺ T cells. Each group of cells prepared from the peripheral blood of donor 1 was mixed with the CD3⁺ T cells prepared from the peripheral blood of donor 2 according to Example 2 in an equal proportion. After 24 h, the expression level of IFN-γ in the cell mixture system was assayed. The results showed that the expression level of IFN-γ in the T-cell group with double-gene knockout was lower than that in the T-cell group with triple-gene knockout.

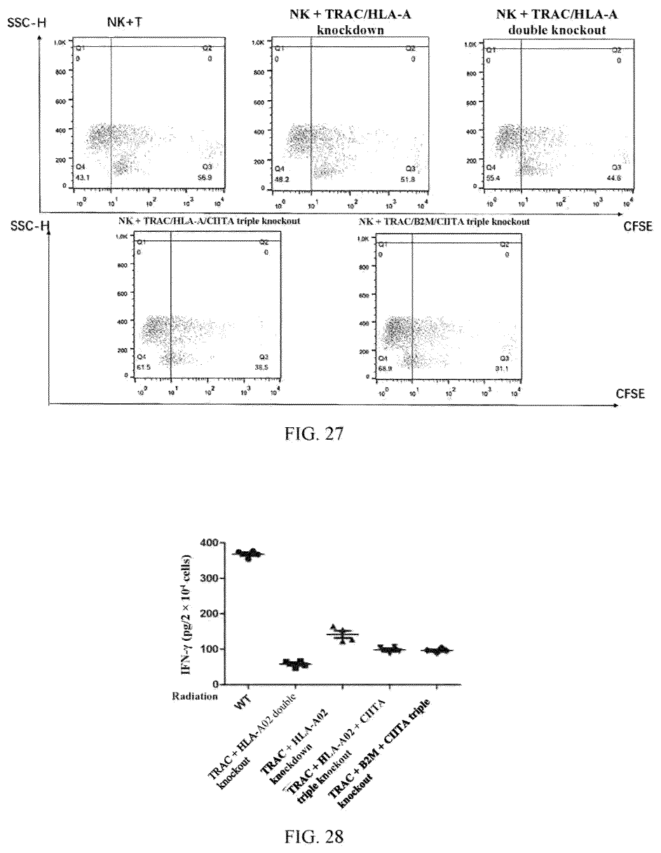

The results are shown in FIG. 28. WT refers to a case without any knockout treatment, TRAC+HLA-A double knockout refers to the result of T cells with double-gene knockout of TRAC and HLA-A genes; TRAC+HLA-A+CIITA triple knockout refers to the result of T cells with triple-gene knockout of TRAC, HLA-A, and CIITA; TRAC+B2M+CIITA triple knockout refers to the result of T cells with triple-gene knockout of B2M, CIITA, and TRAC; and TRAC+HLA-A knockdown refers to the result of T cells with knockdown of TRAC and HLA-A genes prepared in Example 16.

### Example 20. Preparation of CAR-T Cells with Double-Gene Knockout of TRAC Gene and HLA-A Gene, CAR-T Cells with Triple-Gene Knockout of TRAC Gene, HLA-A Gene, and CIITA Gene, and CAR-T Cells with Knockout of TRAC Gene, B2M Gene, and CIITA Gene

(1) CD3⁺ T cells were obtained according to the method in Example 2 (D0) and activated with CD3/CD28 antibody magnetic beads. After activation, lentiviral vectors (lentiviruses comprising CD19-CAR, CD20-CAR, or BCMA-CAR) were transfected on D1, the lentiviral vectors were washed off on D2, CAR-positive T cells were sorted on D3, and the cells were continued to be cultured until D5.

(2) The CAR-T cells obtained on D5 were used as starting cells, and cells with double-gene knockout of the TRAC gene and HLA-A gene, CAR-T cells with triple-gene knockout of the TRAC gene, HLA-A gene, and CIITA gene, and CAR-T cells with triple-gene knockout of the TRAC gene, B2M gene, and CIITA gene were prepared according to the methods in Examples 12 and 14, respectively.

(3) The CAR-T cells with double-gene knockout and triple-gene knockout described above could be obtained by flow cytometry assay, wherein the yield of the CAR-T cells with double-gene knockout was higher than that of the CAR-T cells with triple-gene knockout.

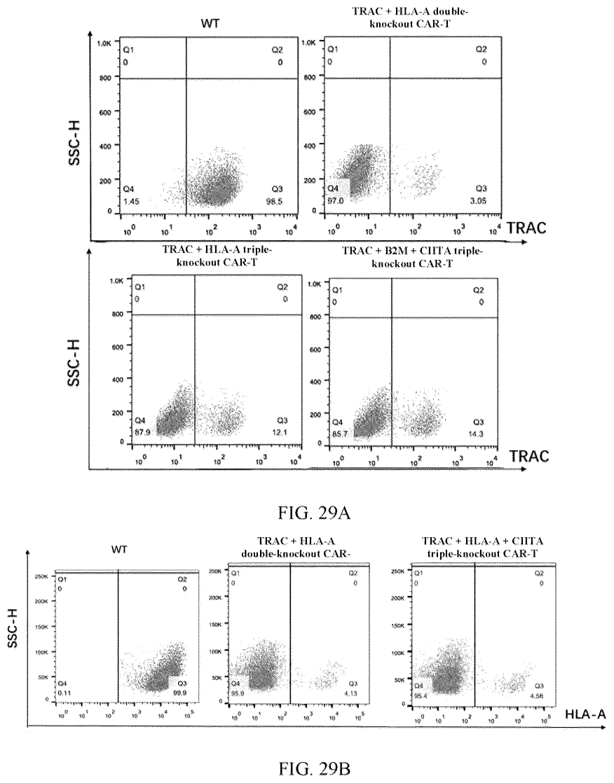

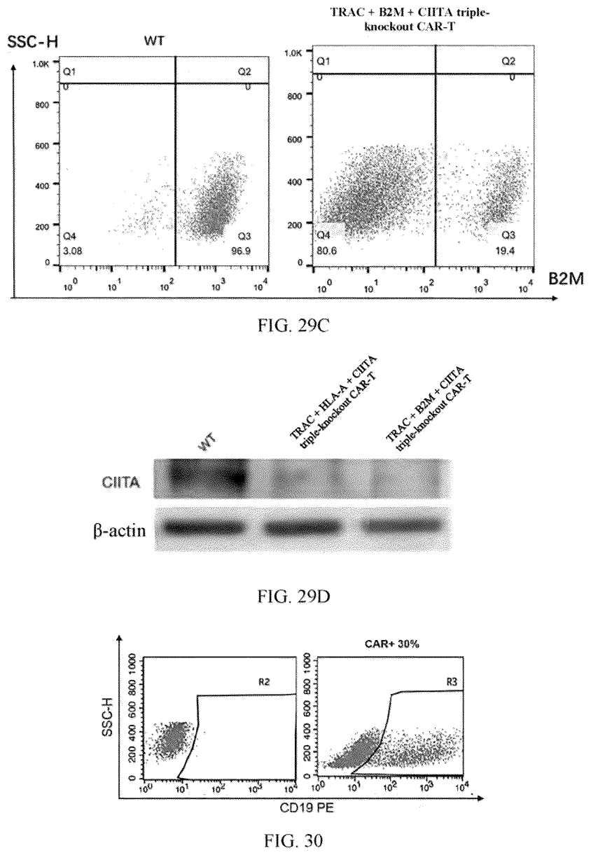

The results are shown in FIGS. 29A-29D. FIGS. 29A-29C show the knockout of TRAC, HLA-A and B2M at protein levels in sequence. FIG. 29D shows the knockout of CIITA at the protein level, wherein WT refers to a case without any knockout treatment, TRAC+HLA-A double knockout refers to the result of CAR-T cells with double-gene knockout of TRAC and HLA-A genes; TRAC+HLA-A+CIITA triple knockout refers to the result of CAR-T cells with triple-gene knockout of TRAC, HLA-A, and CIITA; TRAC+B2M+CIITA triple knockout refers to the result of CAR-T cells with triple-gene knockout of B2M, CIITA, and TRAC.

Among these, the transfection efficiency of CD19CAR is shown in FIGS. 30A-30B, wherein CAR30%+ represented the transfection efficiency of CD19 CAR.

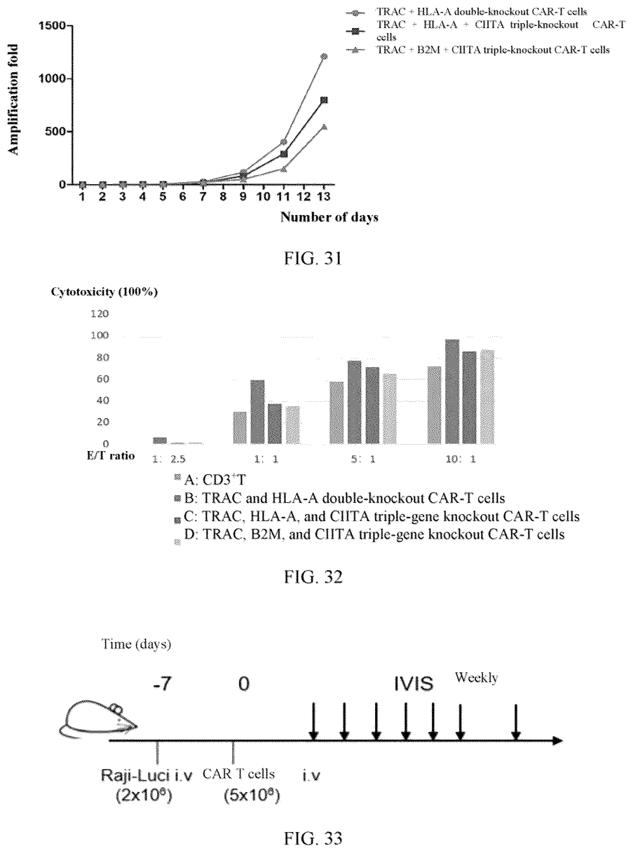

FIG. 31 shows the amplification fold of different cells, wherein the CAR-T cells with double-gene knockout of the TRAC gene and HLA-A gene had the highest amplification fold.

### Example 21. Anti-Tumor Effect of CAR-T Cells with Double-Gene Knockout of TRAC Gene and HLA-A Gene

The CAR-T cells with double knockout of the TRAC gene and HLA-A gene (targeting CD19, CD20, or BCMA) were prepared in Example 21. Target cells expressing the luciferase gene (target gene-positive leukemia or lymphoma cell lines, such as Raji, Jurkat, MMIS, and the like) were seeded to a well plate. The CAR-T cells with double-gene knockout, CAR-T cells with triple-gene knockout, or T cells without gene knockout were added at different effector-to-target ratios (1:2.5, 1:1, 5:1, and 10:1), respectively. After 24 h of co-culture, the cells were transferred to an assay well plate, luciferase substrate was added, and fluorescence value was detected by a microplate reader. Killing efficiency=1−fluorescence value of T cells co-cultured with target cells/fluorescence value of target cells cultured alone.

The results showed that the CAR-T cells with double knockout of the TRAC gene and HLA-A gene had a significant killing effect on tumor cells.

FIG. 32 shows the killing effect on CD19 target cell Raji-Luciferase, wherein the CAR-T cells with double knockout of the TRAC gene and HLA-A gene exhibit the most significant killing effect. At each E/T ratio, the results corresponding to notes A-D were shown from left to right.

### Example 22. Anti-Tumor Effect of CAR-T Cells with Double-Gene Knockout of TRAC Gene and HLA-A Gene

NSG mice were injected with tumor cells intravenously. After the tumor was successfully established, the CAR-T cells with double-gene knockout of the TRAC gene and HLA-A gene, CAR-T cells with triple-gene knockout, and T cells without gene knockout were reinfused to the mice. The tumor volume of the mice was monitored.

The mice to which the CAR-T cells with double-gene knockout were reinfused exhibited a significantly slower growth rate of the tumor.

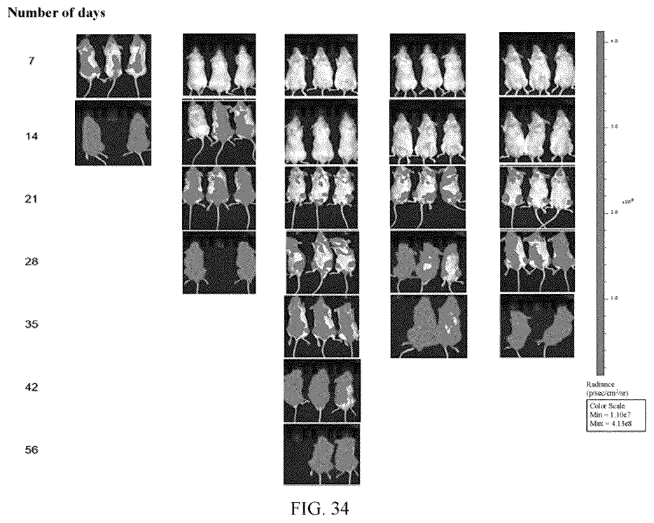

The results are shown in FIGS. 33-34, wherein FIG. 33 showed the administration mode in mice, i.v. represented intravenous injection, CAR-T cells represented CAR-T cells with double-gene knockout and CAR-T cells with triple-gene knockout expressing CD19 CAR. FIG. 34 showed the tumor volume in the mice after the CAR-T cells were administered, wherein FIG. 34 showed, from left column to right column, the tumor volume in the mice after normal saline, unmodified T cells, CD19 CAR-T cells with double-gene knockout of the TRAC gene and HLA-A gene, CD19 CAR-T cells with triple-gene knockout of TRAC, HLA-A, and CIITA, and CD19 CAR-T cells with triple-gene knockout of B2M, CIITA, and TRAC were separately administered in sequence. The results showed that the mice to which the CAR-T cells with double-gene knockout of the TRAC gene and HLA-A gene were reinfused exhibited a significantly slower growth rate of the tumor.

In summary:
1. The present application prepares a chimeric antigen receptor targeting B7H3, an antigen-binding domain of the recombinant receptor is derived from a nanoantibody, and the recombinant receptor has the characteristics of small molecular weight and stable structure.
2. The present application provides a lentiviral expression vector. pCDH-CMV-MCS-EF1-copGFP is used as a backbone, and an ampicillin resistance gene β-lactamase on the vector is replaced with aminoglycoside phosphotransferase derived from Tn5 to enable the vector to have kanamycin resistance; the CMV promoter and its adjacent downstream multiple cloning site, which are potentially threatening in vivo applications, are deleted; the copGFP gene that is started to express by the EFI promoter in the original vector is deleted, a Sall enzyme digestion site is retained, and a Smal enzyme digestion site is added to the 5′ end of Sall for vector construction to form a final target vector.
3. The present application optimizes the protein-RNA complex electrotransfection technology. More than 90% of double-gene knockout efficiency in primary T cells is obtained.
4. In the present application, the donor source is based on HLA-B homozygotes that occur frequently in the population, and one of the alleles of HLA-B in the patient is consistent with the homozygotes in the donor, so that cells from these donors can cover a high number of patient populations, and the rejection response caused by HLA-B can be reduced.
5. According to the present application, HLA-A molecules highly related to rejection are screened out for knockout, and other HLA-I molecules are retained, so that the rejection of allogeneic cells is reduced, the complete knockout of HLA molecules and elimination of HLA molecules by NK cells are avoided, thereby greatly prolonging the half-life of allogeneic CAR-T cells in vivo.
6. The present application firstly constructs anti-B7H3-UCAR-T cells with highly efficient double knockout of TCR and HLA-A, achieves a safe shelf-ready-to-use therapeutic agent, improves the anti-tumor effect, and is used for treating diseases such as adrenocortical carcinoma, bladder cancer, breast cancer, cholangiocarcinoma, colorectal cancer, lymphoma, esophageal cancer, brain glioma, head and neck squamous cell carcinoma, kidney cancer, liver cancer, lung cancer, ovarian cancer, pancreatic cancer, prostate cancer, sarcoma, melanoma, gastric cancer, thymus cancer, and endometrial cancer.

1. An antigen-binding polypeptide, binding B7H3, comprising an antibody heavy chain variable region (VH), wherein the VH comprises:
i) an HCDR1 comprising an amino acid sequence set forth in SEQ ID NO: 2, an HCDR2 comprising an amino acid sequence set forth in SEQ ID NO: 5, and an HCDR3 comprising an amino acid sequence set forth in SEQ ID NO: 8; or
ii) an HCDR1 comprising an amino acid sequence set forth in SEQ ID NO: 3, an HCDR2 comprising an amino acid sequence set forth in SEQ ID NO: 6, and an HCDR3 comprising an amino acid sequence set forth in SEQ ID NO: 9.

2. The antigen-binding polypeptide according to claim 1, wherein the VH comprises an amino acid sequence set forth in SEQ ID NO: 26, SEQ ID NO: 27, SEQ ID NO: 28, or SEQ ID NO: 29.

3-21. (canceled)

22. The antigen-binding polypeptide according to claim 1, wherein the antigen-binding polypeptide further comprises an antibody or an antigen-binding fragment thereof.

23. (canceled)

24. The antigen-binding polypeptide according to claim 22, wherein the antibody is a single-domain antibody (VHH).

25. A chimeric antigen receptor (CAR) comprising a B7H3-targeting moiety, wherein the B7H3-targeting moiety comprises the antigen-binding polypeptide according to claim 1.

26. (canceled)

27. The chimeric antigen receptor according to claim 25, further comprising a transmembrane domain,
wherein the transmembrane domain comprises:
a transmembrane domain derived from a protein selected from the group consisting of CD8A, CD8B, CD28, CD3ε (CD3e), 4-1BB, CD4, CD27, CD7, PD-1, TRAC, TRBC, CD3ζ, CTLA-4, LAG-3, CD5, ICOS, OX40, NKG2D, 2B4 (CD244), FcεRIγ, BTLA, CD30, GITR, HVEM, DAP10, CD2, NKG2C, LIGHT, DAP12, CD40L (CD154), TIM1, CD226, DR3, CD45, CD80, CD86, CD9, CD16, CD22, CD33, CD37, CD64, and SLAM.

28-29. (canceled)

30. The chimeric antigen receptor according to claim 25, further comprising an intracellular co-stimulatory signaling domain,
wherein the intracellular co-stimulatory signaling domain comprises an intracellular co-stimulatory signaling domain derived from a protein selected from the group consisting of CD28, CD137, CD27, CD2, CD7, CD8A, CD8B, OX40, CD226, DR3, SLAM, CDS, ICAM-1, NKG2D, NKG2C, B7H3, 2B4, FcεRIγ, BTLA, GITR, HVEM, DAP10, DAP12, CD30, CD40, CD40L, TIM1, PD-1, LFA-1, LIGHT, JAML, CD244, CD100, ICOS, CD40, and MyD88.

31-32. (canceled)

33. The chimeric antigen receptor according to claim 25, further comprising an intracellular signaling domain,
wherein the intracellular signaling domain comprises:
an intracellular signaling domain derived from a protein selected from the group consisting of CD3ζ, CD3δ, CD3γ, CD3ε, CD79a, CD79b, FceRIγ, FceRIβ, FcγRIIa, bovine leukemia virus gp30, Epstein-Barr virus (EBV) LMP2A, simian immunodeficiency virus PBj14 Nef, DAP10, DAP-12; and
a domain comprising an ITAM.

34-35. (canceled)

36. The chimeric antigen receptor according to claim 27, further comprising a hinge region between the targeting moiety and the transmembrane domain,
wherein the hinge region comprises a hinge region derived from a protein selected from the group consisting of CD28, IgG1, IgG4, IgD, 4-1BB, CD4, CD27, CD7, CD8A, PD-1, ICOS, OX40, NKG2D, NKG2C, FcεRIγ, BTLA, GITR, DAP10, TIM1, SLAM, CD30, and LIGHT.

37-38. (canceled)

39. The chimeric antigen receptor according to claim 25, wherein a non-targeting moiety of the chimeric antigen receptor comprises:
a transmembrane domain of CD8A molecule;
a hinge region of CD8A;
an intracellular co-stimulatory signaling domain of 4-1BB; and
an intracellular signaling domain of CD3ζ.

40-50. (canceled)

51. An immune effector cell expressing the CAR according to claim 25.

52-53. (canceled)

54. The immune effector cell according to claim 51, wherein the immune effector cell further comprises an autologous or a non-autologous immune effector cell.

55. The immune effector cell according to claim 51, wherein the immune effector cell further comprises a modified immune effector cell, and
wherein the modified immune effector cell comprises a cell that reduces immune rejection caused by allogeneic cell therapy.

56. (canceled)

57. The immune effector cell according to claim 55, wherein a function of a T cell antigen receptor (TCR) and a major histocompatibility complex (MHCI, MHCII) in the modified immune effector cell are inhibited in a T cell.

58. The immune effector cell according to claim 55, wherein a modification comprises down-regulation of an expression and/or an activity of an immune rejection-related gene, and
wherein the immune rejection-related gene is selected from the group consisting of a TRAC gene, a TRBC gene, an HLA-A gene, an HLA-B gene, a B2M gene, and a CIITA gene.

59. (canceled)

60. The immune effector cell according to claim 58, wherein the expression and/or the activity of the TRAC gene and the HLA-A gene in the modified immune effector cell is down-regulated as compared to a corresponding unmodified cell.

61-70. (canceled)

71. The immune effector cell according to claim 55, wherein a modification comprises administering to the immune effector cell a substance selected from the group consisting of an antisense RNA, an siRNA, an shRNA, and a CRISPR/Cas9 system.

72. (canceled)

73. The immune effector cell according to claim 71, wherein the modification further comprises administering to the immune effector cell an sgRNA targeting an exon portion of a TRAC gene.

74. The immune effector cell according to claim 73, wherein the sgRNA targeting the exon portion of the TRAC gene comprises a nucleotide sequence set forth in any one of SEQ ID NO: 157 to SEQ ID NO: 171.

75. The immune effector cell according to claim 71, wherein the modification further comprises administering to the immune effector cell an sgRNA targeting an exon portion of a HLA-A gene.

76. The immune effector cell according to claim 75, wherein the sgRNA targeting the exon portion of the HLA-A gene comprises a nucleotide sequence set forth in any one of SEQ ID NO: 172 to SEQ ID NO: 212.

77-79. (canceled)

80. The immune effector cell according to claim 51, wherein the immune effector cell is an HLA-B homozygous cell.

81. (canceled)

82. The immune effector cell according to claim 51, wherein the immune effector cell is an HLA-A homozygous or heterozygous cell.

83-114. (canceled)

115. A pharmaceutical composition comprising:
the antigen-binding polypeptide;
the chimeric antigen receptor; and/or
the immune effector cell according to claim 51; and/or
a pharmaceutically acceptable carrier.

116-126. (canceled)


SEQUENCE LISTING (216 sequences)

SEQ ID NO: 1 - protein, 8 aa, Artificial Sequence
  1A5 HCDR1, hu1A5 HCDR1; 1G7 HCDR1, hu1G7 HCDR1
  misc_feature (8)..(8): Xaa = Ala or Ser
GRIFSNYX

SEQ ID NO: 2 - protein, 8 aa, Artificial Sequence
  1G7 HCDR1, hu1G7 HCDR1
GRIFSNYA

SEQ ID NO: 3 - protein, 8 aa, Artificial Sequence
  1A5 HCDR1, hu1A5 HCDR1
GRIFSNYS

SEQ ID NO: 4 - protein, 8 aa, Artificial Sequence
  1G7 HCDR2, hu1G7 HCDR2; 1A5 HCDR2, hu1A5 HCDR2
  misc_feature (3)..(3): Xaa = Gly or Trp
  misc_feature (8)..(8): Xaa = Thr or Val
IIXSTGTX

SEQ ID NO: 5 - protein, 8 aa, Artificial Sequence
  1G7 HCDR2, hu1G7 HCDR2
IIGSTGTV

SEQ ID NO: 6 - protein, 8 aa, Artificial Sequence
  1A5 HCDR2, hu1A5 HCDR2
IIWSTGTT

SEQ ID NO: 7 - protein, 18 aa, Artificial Sequence
  1G7 HCDR3, hu1G7 HCDR3; 1A5 HCDR3, hu1A5 HCDR3
  misc_feature (5)..(5): Xaa = Lys or Arg
  misc_feature (17)..(17): Xaa = Gly or Val
AASFXYSGIYGREADFXS

SEQ ID NO: 8 - protein, 18 aa, Artificial Sequence
  1G7 HCDR3, hu1G7 HCDR3
AASFRYSGIYGREADFVS

SEQ ID NO: 9 - protein, 18 aa, Artificial Sequence
  1A5 HCDR3, hu1A5 HCDR3
AASFKYSGIYGREADFGS

SEQ ID NO: 10 - protein, 25 aa, Artificial Sequence
  1G7 HFR1; hu1G7 HFR1; 1A5 HFR1; hu1A5 HFR1
  misc_feature (1)..(1): Xaa = Ala or Gln
  misc_feature (2)..(2): Xaa = Ala or Val
  misc_feature (14)..(14): Xaa = Pro or Thr
  misc_feature (24)..(24): Xaa = Ala or Asp
XXQLVESGGGLVQXGGSLRLSCAXS

SEQ ID NO: 11 - protein, 25 aa, Artificial Sequence
  1G7 HFR1
AVQLVESGGGLVQTGGSLRLSCADS

SEQ ID NO: 12 - protein, 25 aa, Artificial Sequence
  hu1G7 HFR1
AVQLVESGGGLVQPGGSLRLSCAAS

SEQ ID NO: 13 - protein, 25 aa, Artificial Sequence
  1A5 HFR1
QAQLVESGGGLVQTGGSLRLSCAAS

SEQ ID NO: 14 - protein, 25 aa, Artificial Sequence
  hu1A5 HFR1
QVQLVESGGGLVQPGGSLRLSCAAS

SEQ ID NO: 15 - protein, 17 aa, Artificial Sequence
  1G7 HFR2, hu1G7 HFR2; 1A5  HFR2, hu1A5 HFR2
  misc_feature (1)..(1): Xaa = Met or Val
XGWFRQAPGKEREFVAA

SEQ ID NO: 16 - protein, 17 aa, Artificial Sequence
  1G7 HFR2, hu1G7 HFR2
MGWFRQAPGKEREFVAA

SEQ ID NO: 17 - protein, 17 aa, Artificial Sequence
  1A5 HFR2, hu1A5 HFR2
VGWFRQAPGKEREFVAA

SEQ ID NO: 18 - protein, 38 aa, Artificial Sequence
  1G7 HFR3; hu1G7 HFR3; hu1A5 HFR3; 1A5 HFR3
  misc_feature (1)..(1): Xaa = Asn or Tyr
  misc_feature (2)..(2): Xaa = Leu or Tyr
  misc_feature (5)..(5): Xaa = Pro or Ser
  misc_feature (6)..(6): Xaa = Met or Val
  misc_feature (17)..(17): Xaa = Ala or Ser
  misc_feature (21)..(21): Xaa = Leu or Val
  misc_feature (22)..(22): Xaa = Val or Tyr
  misc_feature (29)..(29): Xaa = Lys or Arg
  misc_feature (30)..(30): Xaa = Ala or Pro
  misc_feature (35)..(35): Xaa = Ile or Val
XXADXXKGRFTISRDNXKNTXXLQMNSLXXEDTAXYYC

SEQ ID NO: 19 - protein, 38 aa, Artificial Sequence
  1G7 HFR3
NYADSMKGRFTISRDNAKNTVVLQMNSLKPEDTAIYYC

SEQ ID NO: 20 - protein, 38 aa, Artificial Sequence
  hu1G7 HFR3; hu1A5 HFR3
YYADSVKGRFTISRDNSKNTLYLQMNSLRAEDTAVYYC

SEQ ID NO: 21 - protein, 38 aa, Artificial Sequence
  1A5 HFR3
NLADPMKGRFTISRDNAKNTVVLQMNSLKPEDTAIYYC

SEQ ID NO: 22 - protein, 11 aa, Artificial Sequence
  1G7 HFR4; 1A5 HFR4; hu1G7 HFR4; hu1A5 HFR4
  misc_feature (6)..(6): Xaa = Leu or Gln
WGQGTXVTVSS

SEQ ID NO: 23 - protein, 11 aa, Artificial Sequence
  1G7 HFR4; 1A5 HFR4
WGQGTQVTVSS

SEQ ID NO: 24 - protein, 11 aa, Artificial Sequence
  hu1G7 HFR4; hu1A5 HFR4
WGQGTLVTVSS

SEQ ID NO: 25 - protein, 125 aa, Artificial Sequence
  1G7(antibody variable region); hu1G7(antibody variable region); 1A5(antibody variable region); hu1A5(antibody variable region)
  misc_feature (1)..(1): Xaa = Ala or Gln
  misc_feature (2)..(2): Xaa = Ala or Val
  misc_feature (14)..(14): Xaa = Pro or Thr
  misc_feature (24)..(24): Xaa = Ala or Asp
  misc_feature (33)..(33): Xaa = Ala or Ser
  misc_feature (34)..(34): Xaa = Met or Val
  misc_feature (53)..(53): Xaa = Gly or Trp
  misc_feature (58)..(58): Xaa = Thr or Val
  misc_feature (59)..(59): Xaa = Asn or Tyr
  misc_feature (60)..(60): Xaa = Leu or Tyr
  misc_feature (63)..(63): Xaa = Pro or Ser
  misc_feature (64)..(64): Xaa = Met or Val
  misc_feature (75)..(75): Xaa = Ala or Ser
  misc_feature (79)..(79): Xaa = Leu or Val
  misc_feature (80)..(80): Xaa = Val or Tyr
  misc_feature (87)..(87): Xaa = Lys or Arg
  misc_feature (88)..(88): Xaa = Ala or Pro
  misc_feature (93)..(93): Xaa = Ile or Val
  misc_feature (101)..(101): Xaa = Lys or Arg
  misc_feature (113)..(113): Xaa = Gly or Val
  misc_feature (120)..(120): Xaa = Leu or Gln
XXQLVESGGGLVQXGGSLRLSCAXSGRIFSNYXXGWFRQAPGKEREFVAAIIXSTGTXXXADXXKGRFTISRDNXKNTXXLQMNSLXXEDTAXYYCAASFXYSGIYGREADFXSWGQGTXVTVSS

SEQ ID NO: 26 - protein, 125 aa, Artificial Sequence
  1G7(antibody variable region)
AVQLVESGGGLVQTGGSLRLSCADSGRIFSNYAMGWFRQAPGKEREFVAAIIGSTGTVNYADSMKGRFTISRDNAKNTVVLQMNSLKPEDTAIYYCAASFRYSGIYGREADFVSWGQGTQVTVSS

SEQ ID NO: 27 - protein, 125 aa, Artificial Sequence
  hu1G7(antibody variable region)
AVQLVESGGGLVQPGGSLRLSCAASGRIFSNYAMGWFRQAPGKEREFVAAIIGSTGTVYYADSVKGRFTISRDNSKNTLYLQMNSLRAEDTAVYYCAASFRYSGIYGREADFVSWGQGTLVTVSS

SEQ ID NO: 28 - protein, 125 aa, Artificial Sequence
  1A5(antibody variable region)
QAQLVESGGGLVQTGGSLRLSCAASGRIFSNYSVGWFRQAPGKEREFVAAIIWSTGTTNLADPMKGRFTISRDNAKNTVVLQMNSLKPEDTAIYYCAASFKYSGIYGREADFGSWGQGTQVTVSS

SEQ ID NO: 29 - protein, 125 aa, Artificial Sequence
  hu1A5(antibody variable region)
QVQLVESGGGLVQPGGSLRLSCAASGRIFSNYSVGWFRQAPGKEREFVAAIIWSTGTTYYADSVKGRFTISRDNSKNTLYLQMNSLRAEDTAVYYCAASFKYSGIYGREADFGSWGQGTLVTVSS

SEQ ID NO: 30 - protein, 237 aa, Artificial Sequence
  CAR Amino acid sequence of the non-antigen binding domain
FVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 31 - protein, 21 aa, Artificial Sequence
  CD8A signal peptide amino acid sequence
MALPVTALLLPLALLLHAARP

SEQ ID NO: 32 - protein, 383 aa, Artificial Sequence
  1G7 CAR VHH
MALPVTALLLPLALLLHAARPAVQLVESGGGLVQTGGSLRLSCADSGRIFSNYAMGWFRQAPGKEREFVAAIIGSTGTVNYADSMKGRFTISRDNAKNTVVLQMNSLKPEDTAIYYCAASFRYSGIYGREADFVSWGQGTQVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 33 - protein, 383 aa, Artificial Sequence
  Hu1G7 CAR VHH
MALPVTALLLPLALLLHAARPAVQLVESGGGLVQPGGSLRLSCAASGRIFSNYAMGWFRQAPGKEREFVAAIIGSTGTVYYADSVKGRFTISRDNSKNTLYLQMNSLRAEDTAVYYCAASFRYSGIYGREADFVSWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 383 aa, Artificial Sequence
  1A5 CAR VHH
MALPVTALLLPLALLLHAARPQAQLVESGGGLVQTGGSLRLSCAASGRIFSNYSVGWFRQAPGKEREFVAAIIWSTGTTNLADPMKGRFTISRDNAKNTVVLQMNSLKPEDTAIYYCAASFKYSGIYGREADFGSWGQGTQVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35 - protein, 383 aa, Artificial Sequence
  Hu1A5 CAR VHH
MALPVTALLLPLALLLHAARPQVQLVESGGGLVQPGGSLRLSCAASGRIFSNYSVGWFRQAPGKEREFVAAIIWSTGTTYYADSVKGRFTISRDNSKNTLYLQMNSLRAEDTAVYYCAASFKYSGIYGREADFGSWGQGTLVTVSSFVPVFLPAKPTTTPAPRPPTPAPTIASQPLSLRPEACRPAAGGAVHTRGLDFACDIYIWAPLAGTCGVLLLSLVITLYCNHRNKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 36 - DNA, 375 bp, Artificial Sequence
  1G7(antibody variable region) nucleotide sequence
gctgtccagcttgtggagtctggggggggcctcgtccagacaggcggttctctccgcctctcctgtgctgattctggcagaatcttttcaaactacgcaatgggctggttcaggcaggcaccaggcaaggagcgggagttcgtggctgctatcattggatctactggcaccgttaactacgcagacagcatgaaaggcagatttaccatctcaagagataacgccaaaaatactgtagtcctccagatgaactctctgaagcctgaggataccgctatatattattgtgcggcgtcattcaggtacagcggcatctatggccgcgaagcggattttgttagttggggacaagggacccaagtgacagtgtcttca

SEQ ID NO: 37 - DNA, 375 bp, Artificial Sequence
  hu1G7(antibody variable region) nucleotide sequence
gctgttcagctggtggagtctggtggcgggctcgttcagccgggcggcagcctcagactgagctgtgcagcctccggaaggatcttttcaaattacgcaatggggtggtttaggcaggcaccaggaaaggagcgagagtttgtggcagcaataatcggttctaccggcacagtgtattacgctgatagcgtgaaaggcaggttcaccatttcaagggataacagtaaaaacacactatacttacagatgaactccttacgagccgaagacacggcggtgtactactgtgccgcatcctttagatactctggaatttacggacgcgaggccgactttgtcagctggggtcaggggactcttgttaccgtatcctct

SEQ ID NO: 38 - DNA, 375 bp, Artificial Sequence
  1A5(antibody variable region) nucleotide sequence
caggctcaactggtggagtcgggaggtggcttggtgcagacaggagggagcctgcgactctcctgtgcggcttccggaaggattttctctaattattctgtcggatggttccggcaggccccaggcaaagagcgtgaattcgttgcggcgataatctggtcgaccggaacaacgaatctggccgatcccatgaaggggagattcaccattagtagagacaacgcaaaaaacacagtagtcctgcagatgaacagcctcaaacccgaggatacagctatctattactgcgcagcttcctttaaatattctggcatatatggcagagaagcagattttgggagttgggggcagggcacccaggtgacagtcagttct

SEQ ID NO: 39 - DNA, 375 bp, Artificial Sequence
  hu1A5(antibody variable region) nucleotide sequence
caggtccaattagtggaaagtggtgggggattagtgcaaccgggcggctccctccgcctttcctgcgccgcttctggcagaatcttctccaactactccgttggatggtttcgtcaggccccaggaaaagagagagaattcgtggctgctatcatatggagcacggggaccacctactatgccgatagcgtcaaaggcaggttcactatcagcagggataatagcaagaatacactatacttgcaaatgaactcgctgcgagccgaagacacagccgtctactactgtgcggcgtcatttaaatacagcggcatttacggccgagaggcagatttcggaagttgggggcagggaaccttggtgacagtctcctcg

SEQ ID NO: 40 - DNA, 711 bp, Artificial Sequence
  CAR non-antigen-binding DNA sequence
ttcgtccccgtgttcctgcctgccaagccaacaactacccctgctccacgaccacctactccagcacctaccatcgcaagtcagcccctgtcactgcgacctgaggcttgccggccagcagctggaggagcagtgcacacccgaggcctggacttcgcatgcgatatctacatttgggcaccactggctggaacctgtggggtcctgctgctgagcctggtcatcaccctgtattgtaaccacagaaataaaagggggcgcaagaaactgctgtacatcttcaagcagccttttatgcgcccagtgcagacaactcaggaggaagacggatgctcttgtcggttcccagaggaggaggaaggaggctgcgagctgagagtgaagttcagccggagcgccgatgcaccagcatatcagcagggacagaatcagctgtacaacgagctgaatctgggcaggcgcgaggaatatgacgtgctggataagcgacgaggacgggaccccgaaatgggaggaaaacccagaaggaagaaccctcaggaggggctgtataatgaactgcagaaagacaagatggctgaggcatacagcgaaattggaatgaaaggagagcgccgacgggggaagggacacgatgggctgtaccagggactgtcaaccgccactaaagatacctacgacgcactgcacatgcaggctctgcccccaaga

SEQ ID NO: 41 - DNA, 63 bp, Artificial Sequence
  CD8A signal peptide nucleic acid sequence
atggccttaccagtgaccgccttgctcctgccgctggccttgctgctccacgccgccaggccg

SEQ ID NO: 42 - protein, 21 aa, Artificial Sequence
  CD8A transmembrane domain
IYIWAPLAGTCGVLLLSLVIT

SEQ ID NO: 43 - protein, 21 aa, Artificial Sequence
  CD8B transmembrane domain
ITLGLLVAGVLVLLVSLGVAI

SEQ ID NO: 44 - protein, 27 aa, Artificial Sequence
  CD28 transmembrane domain
FWVLVVVGGVLACYSLLVTVAFIIFWV

SEQ ID NO: 45 - protein, 27 aa, Artificial Sequence
  4-1BB transmembrane domain
IISFFLALTSTALLFLLFFLTLRFSVV

SEQ ID NO: 46 - protein, 22 aa, Artificial Sequence
  CD4 transmembrane domain
MALIVLGGVAGLLLFIGLGIFF

SEQ ID NO: 47 - protein, 21 aa, Artificial Sequence
  CD27 transmembrane domain
ILVIFSGMFLVFTLAGALFLH

SEQ ID NO: 48 - protein, 21 aa, Artificial Sequence
  CD7 transmembrane domain
AALAVISFLLGLGLGVACVLA

SEQ ID NO: 49 - protein, 21 aa, Artificial Sequence
  PD-1 transmembrane domain
VGVVGGLLGSLVLLVWVLAVI

SEQ ID NO: 50 - protein, 23 aa, Artificial Sequence
  TRAC transmembrane domain
VIGFRILLLKVAGFNLLMTLRLW

SEQ ID NO: 51 - protein, 21 aa, Artificial Sequence
  TRBC transmembrane domain
ILLGKATLYAVLVSALVLMAM

SEQ ID NO: 52 - protein, 26 aa, Artificial Sequence
  CD3epsilon transmembrane domain
VMSVATIVIVDICITGGLLLLVYYWS

SEQ ID NO: 53 - protein, 21 aa, Artificial Sequence
  CD3zeta transmembrane domain
LCYLLDGILFIYGVILTALFL

SEQ ID NO: 54 - protein, 21 aa, Artificial Sequence
  CTLA-4 transmembrane domain
FLLWILAAVSSGLFFYSFLLT

SEQ ID NO: 55 - protein, 21 aa, Artificial Sequence
  LAG-3 transmembrane domain
LLFLILGVLSLLLLVTGAFGF

SEQ ID NO: 56 - protein, 30 aa, Artificial Sequence
  CD5 transmembrane domain
AGLAAGTVASIILALVLLVVLLVVCGPLAY

SEQ ID NO: 57 - protein, 21 aa, Artificial Sequence
  ICOS transmembrane domain
FWLPIGCAAFVVVCILGCILI

SEQ ID NO: 58 - protein, 21 aa, Artificial Sequence
  OX40 transmembrane domain
VAAILGLGLVLGLLGPLAILL

SEQ ID NO: 59 - protein, 21 aa, Artificial Sequence
  NKG2D transmembrane domain
PFFFCCFIAVAMGIRFIIMVA

SEQ ID NO: 60 - protein, 21 aa, Artificial Sequence
  2B4 transmembrane domain
FLVIIVILSALFLGTLACFCV

SEQ ID NO: 61 - protein, 21 aa, Artificial Sequence
  FcepsilonRIgamma transmembrane domain
LCYILDAILFLYGIVLTLLYC

SEQ ID NO: 62 - protein, 21 aa, Artificial Sequence
  BTLA transmembrane domain
LLPLGGLPLLITTCFCLFCCL

SEQ ID NO: 63 - protein, 21 aa, Artificial Sequence
  CD30 transmembrane domain
PVLFWVILVLVVVVGSSAFLL

SEQ ID NO: 64 - protein, 21 aa, Artificial Sequence
  GITR transmembrane domain
LGWLTVVLLAVAACVLLLTSA

SEQ ID NO: 65 - protein, 21 aa, Artificial Sequence
  HVEM transmembrane domain
WWFLSGSLVIVIVCSTVGLII

SEQ ID NO: 66 - protein, 21 aa, Artificial Sequence
  DAP10 transmembrane domain
LLAGLVAADAVASLLIVGAVF

SEQ ID NO: 67 - protein, 26 aa, Artificial Sequence
  CD2 transmembrane domain
IYLIIGICGGGSLLMVFVALLVFYIT

SEQ ID NO: 68 - protein, 23 aa, Artificial Sequence
  NKG2C transmembrane domain
LTAEVLGIICIVLMATVLKTIVL

SEQ ID NO: 69 - protein, 21 aa, Artificial Sequence
  LIGHT transmembrane domain
VGLGLLLLLMGAGLAVQGWFL

SEQ ID NO: 70 - protein, 21 aa, Artificial Sequence
  DAP12 transmembrane domain
GVLAGIVMGDLVLTVLIALAV

SEQ ID NO: 71 - protein, 24 aa, Artificial Sequence
  CD40L transmembrane domain
IFMYLLTVFLITQMIGSALFAVYL

SEQ ID NO: 72 - protein, 21 aa, Artificial Sequence
  TIM1 transmembrane domain
IYAGVCISVLVLLALLGVIIA

SEQ ID NO: 73 - protein, 21 aa, Artificial Sequence
  CD226 transmembrane domain
GGTVLLLLFVISITTIIVIFL

SEQ ID NO: 74 - protein, 21 aa, Artificial Sequence
  DR3 transmembrane domain
MFWVQVLLAGLVVPLLLGATL

SEQ ID NO: 75 - protein, 21 aa, Artificial Sequence
  CD45 transmembrane domain
ALIAFLAFLIIVTSIALLVVL

SEQ ID NO: 76 - protein, 21 aa, Artificial Sequence
  CD80 transmembrane domain
LLPSWAITLISVNGIFVICCL

SEQ ID NO: 77 - protein, 21 aa, Artificial Sequence
  CD86 transmembrane domain
WITAVLPTVIICVMVFCLILW

SEQ ID NO: 78 - protein, 21 aa, Artificial Sequence
  CD9 transmembrane domain1
LLFGFNFIFWLAGIAVLAIGL

SEQ ID NO: 79 - protein, 21 aa, Artificial Sequence
  CD9 transmembrane domain2
FYTGVYILIGAGALMMLVGFL

SEQ ID NO: 80 - protein, 24 aa, Artificial Sequence
  CD9 transmembrane domain3
MLGLFFGFLLVIFAIEIAAAIWGY

SEQ ID NO: 81 - protein, 26 aa, Artificial Sequence
  CD9 transmembrane domain4
IGAVGIGIAVVMIFGMIFSMILCCAI

SEQ ID NO: 82 - protein, 21 aa, Artificial Sequence
  CD16 transmembrane domain
VSFCLVMVLLFAVDTGLYFSV

SEQ ID NO: 83 - protein, 19 aa, Artificial Sequence
  CD22 transmembrane domain
VAVGLGSCLAILILAICGL

SEQ ID NO: 84 - protein, 23 aa, Artificial Sequence
  CD33 transmembrane domain
GAIGGAGVTALLALCLCLIFFIV

SEQ ID NO: 85 - protein, 21 aa, Artificial Sequence
  CD37 transmembrane domain1
FNLFFFVLGSLIFCFGIWILI

SEQ ID NO: 86 - protein, 15 aa, Artificial Sequence
  CD37 transmembrane domain2
VLAISGIFTMGIALL

SEQ ID NO: 87 - protein, 26 aa, Artificial Sequence
  CD37 transmembrane domain3
LLGLYFGMLLLLFATQITLGILISTQ

SEQ ID NO: 88 - protein, 25 aa, Artificial Sequence
  CD37 transmembrane domain4
LISIVGICLGVGLLELGFMTLSIFL

SEQ ID NO: 89 - protein, 21 aa, Artificial Sequence
  CD64 transmembrane domain
VLFYLAVGIMFLVNTVLWVTI

SEQ ID NO: 90 - protein, 21 aa, Artificial Sequence
  SLAM transmembrane domain
WAVYAGLLGGVIMILIMVVIL

SEQ ID NO: 91 - protein, 41 aa, Artificial Sequence
  CD28 intracellular co-stimulatory signaling domain
RSKRSRLLHSDYMNMTPRRPGPTRKHYQPYAPPRDFAAYRS

SEQ ID NO: 92 - protein, 42 aa, Artificial Sequence
  CD137 intracellular co-stimulatory signaling domain
KRGRKKLLYIFKQPFMRPVQTTQEEDGCSCRFPEEEEGGCEL

SEQ ID NO: 93 - protein, 48 aa, Artificial Sequence
  CD27 intracellular co-stimulatory signaling domain
QRRKYRSNKGESPVEPAEPCHYSCPREEEGSTIPIQEDYRKPEPACSP

SEQ ID NO: 94 - protein, 116 aa, Artificial Sequence
  CD2 intracellular co-stimulatory signaling domain
KRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 95 - protein, 39 aa, Artificial Sequence
  CD7 intracellular co-stimulatory signaling domain
RTQIKKLCSWRDKNSAACVVYEDMSHSRCNTLSSPNQYQ

SEQ ID NO: 96 - protein, 32 aa, Artificial Sequence
  CD8A intracellular co-stimulatory signaling domain
LYCNHRNRRRVCKCPRPVVKSGDKPSLSARYV

SEQ ID NO: 97 - protein, 19 aa, Artificial Sequence
  CD8B intracellular co-stimulatory signaling domain
HLCCRRRRARLRFMKQFYK

SEQ ID NO: 98 - protein, 42 aa, Artificial Sequence
  OX40 intracellular co-stimulatory signaling domain
ALYLLRRDQRLPPDAHKPPGGGSFRTPIQEEQADAHSTLAKI

SEQ ID NO: 99 - protein, 61 aa, Artificial Sequence
  CD226 intracellular co-stimulatory signaling domain
NRRRRRERRDLFTESWDTQKAPNNYRSPISTSQPTNQSMDDTREDIYVNYPTFSRRPKTRV

SEQ ID NO: 100 - protein, 197 aa, Artificial Sequence
  DR3 intracellular co-stimulatory signaling domain
TYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 101 - protein, 77 aa, Artificial Sequence
  SLAM intracellular co-stimulatory signaling domain
QLRRRGKTNHYQTTVEKKSLTIYAQVQKPGPLQKKLDSFPAQDPCTTIYVAATEPVPESVQETNSITVYASVTLPES

SEQ ID NO: 102 - protein, 29 aa, Artificial Sequence
  ICAM-1 intracellular co-stimulatory signaling domain
NRQRKIKKYRLQQAQKGTPMKPNTQATPP

SEQ ID NO: 103 - protein, 51 aa, Artificial Sequence
  NKG2D intracellular co-stimulatory signaling domain
MGWIRGRRSRHSWEMSEFHNYNLDLKKSDFSTRWQKQRCPVVKSKCRENAS

SEQ ID NO: 104 - protein, 70 aa, Artificial Sequence
  NKG2C intracellular co-stimulatory signaling domain
MSKQRGTFSEVSLAQDPKRQQRKPKGNKSSISGTEQEIFQVELNLQNPSLNHQGIDKIYDCQGLLPPPEK

SEQ ID NO: 105 - protein, 47 aa, Artificial Sequence
  B7-H3 intracellular co-stimulatory signaling domain
CWRKIKQSCEEENAGAEDQDGEGEGSKTALQPLKHSDSKEDDGQEIA

SEQ ID NO: 106 - protein, 120 aa, Artificial Sequence
  2B4 intracellular co-stimulatory signaling domain
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 107 - protein, 42 aa, Artificial Sequence
  FcepsilonRIgamma intracellular co-stimulatory signaling domain; FcepsilonRIgamma intracellular signaling domain
RLKIQVRKAAITSYEKSDGVYTGLSTRNQETYETLKHEKPPQ

SEQ ID NO: 108 - protein, 111 aa, Artificial Sequence
  BTLA intracellular co-stimulatory signaling domain
RRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 109 - protein, 58 aa, Artificial Sequence
  GITR intracellular co-stimulatory signaling domain
QLGLHIWQLRSQCMWPRETQLLLEVPPSTEDARSCQFPEEERGERSAEEKGRLGDLWV

SEQ ID NO: 110 - protein, 60 aa, Artificial Sequence
  HVEM intracellular co-stimulatory signaling domain
CVKRRKPRGDVVKVIVSVQRKRQEAEGEATVIEALQAPPDVTTVAVEETIPSFTGRSPNH

SEQ ID NO: 111 - protein, 24 aa, Artificial Sequence
  DAP10 intracellular co-stimulatory signaling domain; DAP10 intracellular signaling domain
LCARPRRSPAQEDGKVYINMPGRG

SEQ ID NO: 112 - protein, 52 aa, Artificial Sequence
  DAP12 intracellular co-stimulatory signaling domain; DAP-12 intracellular signaling domain
YFLGRLVPRGRGAAEAATRKQRITETESPYQELQGQRSDVYSDLNTQRPYYK

SEQ ID NO: 113 - protein, 189 aa, Artificial Sequence
  CD30 intracellular co-stimulatory signaling domain
CHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 114 - protein, 62 aa, Artificial Sequence
  CD40 intracellular co-stimulatory signaling domain
KKVAKKPTNKAPHPKQEPQEINFPDDLPGSNTAAPVQETLHGCQPVTQEDGKESRISVQERQ

SEQ ID NO: 115 - protein, 22 aa, Artificial Sequence
  CD40L intracellular co-stimulatory signaling domain
MIETYNQTSPRSAATGLPISMK

SEQ ID NO: 116 - protein, 48 aa, Artificial Sequence
  TIM1 intracellular co-stimulatory signaling domain
KKYFFKKEVQQLSVSFSSLQIKALQNAVEKEVQAEDNIYIENSLYATD

SEQ ID NO: 117 - protein, 97 aa, Artificial Sequence
  PD-1 intracellular co-stimulatory signaling domain
CSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 118 - protein, 59 aa, Artificial Sequence
  LFA-1 intracellular co-stimulatory signaling domain
YKVGFFKRNLKEKMEAGRGVPNGIPAEDSEQLASGQEAGDPGCLKPLHEKDSESGGGKD

SEQ ID NO: 119 - protein, 182 aa, Artificial Sequence
  LIGHT intracellular co-stimulatory signaling domain
LQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 120 - protein, 98 aa, Artificial Sequence
  JAML intracellular co-stimulatory signaling domain
KKTCGNKSSVNSTVLVKNTKKTNPEIKEKPCHFERCEGEKHIYSPIIVREVIEEEEPSEKSEATYMTMHPVWPSLRSDRNNSLEKKSGGGMPKTQQAF

SEQ ID NO: 121 - protein, 107 aa, Artificial Sequence
  CD100 intracellular co-stimulatory signaling domain
YKGYLPRQCLKFRSALLIGKKKPKSDFCDREQSLKETLVEPGSFSQQNGEHPKPALDTGYETEQDTITSKVPTDREDSQRIDDLSARDKPFDVKCELKFADSDADGD

SEQ ID NO: 122 - protein, 38 aa, Artificial Sequence
  ICOS intracellular co-stimulatory signaling domain
CWLTKKKYSSSVHDPNGEYMFMRAVNTAKKSRLTDVTL

SEQ ID NO: 123 - protein, 296 aa, Artificial Sequence
  MyD88 intracellular co-stimulatory signaling domain
MAAGGPGAGSAAPVSSTSSLPLAALNMRVRRRLSLFLNVRTQVAADWTALAEEMDFEYLEIRQLETQADPTGRLLDAWQGRPGASVGRLLELLTKLGRDDVLLELGPSIEEDCQKYILKQQQEEAEKPLQVAAVDSSVPRTAELAGITTLDDPLGHMPERFDAFICYCPSDIQFVQEMIRQLEQTNYRLKLCVSDRDVLPGTCVWSIASELIEKRCRRMVVVVSDDYLQSKECDFQTKFALSLSPGAHQKRLIPIKYKAMKKEFPSILRFITVCDYTNPCTKSWFWTRLAKALSLP

SEQ ID NO: 124 - protein, 113 aa, Artificial Sequence
  CD3zeta intracellular signaling domain
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 125 - protein, 45 aa, Artificial Sequence
  CD3delta intracellular signaling domain
GHETGRLSGAADTQALLRNDQVYQPLRDRDDAQYSHLGGNWARNK

SEQ ID NO: 126 - protein, 45 aa, Artificial Sequence
  CD3gamma intracellular signaling domain
GQDGVRQSRASDKQTLLPNDQLYQPLKDREDDQYSHLQGNQLRRN

SEQ ID NO: 127 - protein, 55 aa, Artificial Sequence
  CD3epsilon intracellular signaling domain
KNRKAKAKPVTRGAGAGGRQRGQNKERPPPVPNPDYEPIRKGQRDLYSGLNQRRI

SEQ ID NO: 128 - protein, 61 aa, Artificial Sequence
  CD79a intracellular signaling domain
RKRWQNEKLGLDAGDEYEDENLYEGLNLDDCSMYEDISRGLQGTYQDVGSLNIGDVQLEKP

SEQ ID NO: 129 - protein, 49 aa, Artificial Sequence
  CD79b intracellular signaling domain
LDKDDSKAGMEEDHTYEGLDIDQTATYEDIVTLRTGEVKWSVGEHPGQE

SEQ ID NO: 130 - protein, 44 aa, Artificial Sequence
  FcepsilonRIbeta intracellular signaling domain
ICGAGEELKGNKVPEDRVYEELNIYSATYSELEDPGEMSPPIDL

SEQ ID NO: 131 - protein, 77 aa, Artificial Sequence
  FcgammaRIIa intracellular signaling domain
CRKKRISANSTDPVKAAQFEPPGRQMIAIRKRQLEETNNDYETADGGYMTLNPRAPTDDDKNIYLTLPPNDHVNSNN

SEQ ID NO: 132 - protein, 58 aa, Artificial Sequence
  bovine leukemia virus gp30 intracellular signaling domain
KCLTSRLLKLLRQAPHFPEISFPPKPDSDYQALLPSAPEIYSHLSPTKPDYINLRPCP

SEQ ID NO: 133 - protein, 123 aa, Artificial Sequence
  Epstein-Barr Virus (EBV) LMP2A intracellular signaling domain
MGSLEMVPMGAGPPSPGGDPDGYDGGNNSQYPSASGSSGNTPTPPNDEERESNEEPPPPYEDPYWGNGDRHSDYQPLGTQDQSLYLGLQHDGNDGLPPPPYSPRDDSSQHIYEEAGRGSMNPV

SEQ ID NO: 134 - protein, 260 aa, Artificial Sequence
  Simian immunodeficiency virus PBj14 Nef intracellular signaling domain
GGVTSKKQRRRGGNLYERLLQARGETYGRLWEGLEGEYSQSQDASGKGLSSLSCEPQKYCEGQFMNTPWRNPATERAKLDYRQQNMDDVDSADLVGCPVSPRVPVRIMTYKLAIDMSHFIKEKGGLEGIYYSDRRHKILDLYLEKEEGIIPDWQNYTAGPGIRYPMFFGWLWKLVPVNVSDEAQEDETHYLMHPAQTSQWDDPWGEVLAWKFDPKLAYNYKAFVEHPEEFGSQSGLSKEEVQRRLTARGLLKMADKKKTS

SEQ ID NO: 135 - protein, 134 aa, Artificial Sequence
  CD28  hinge region
NKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKP

SEQ ID NO: 136 - protein, 220 aa, Artificial Sequence
  IgG1 hinge region
PCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 217 aa, Artificial Sequence
  IgG4 hinge region
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 138 - protein, 223 aa, Artificial Sequence
  IgD hinge region
CPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAQAPVKLSLNLLASSDPPEAASWLLCEVSGFSPPNILLMWLEDQREVNTSGFAPARPPPQPRSTTFWAWSVLRVPAPPSPQPATYTCVVSHEDSRTLLNASRSLEVSYVTDHGPMK

SEQ ID NO: 139 - protein, 163 aa, Artificial Sequence
  4-1BB hinge region
LQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQ

SEQ ID NO: 140 - protein, 371 aa, Artificial Sequence
  CD4 hinge region
KKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQP

SEQ ID NO: 141 - protein, 172 aa, Artificial Sequence
  CD27 hinge region
ATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIR

SEQ ID NO: 142 - protein, 155 aa, Artificial Sequence
  CD7 hinge region
AQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALP

SEQ ID NO: 143 - protein, 161 aa, Artificial Sequence
  CD8A hinge region
SQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACD

SEQ ID NO: 144 - protein, 147 aa, Artificial Sequence
  PD-1 hinge region
FLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLV

SEQ ID NO: 145 - protein, 120 aa, Artificial Sequence
  ICOS hinge region
EINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLK

SEQ ID NO: 146 - protein, 133 aa, Artificial Sequence
  OX40 hinge region
QVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 147 - protein, 144 aa, Artificial Sequence
  NKG2D hinge region
IWSAVFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 148 - protein, 138 aa, Artificial Sequence
  NKG2C hinge region
IPFLEQNNSSPNTRTQKARHCGHCPEEWITYSNSCYYIGKERRTWEESLLACTSKNSSLLSIDNEEEMKFLASILPSSWIGVFRNSSHHPWVTINGLAFKHKIKDSDNAELNCAVLQVNRLKSAQCGSSMIYHCKHKL

SEQ ID NO: 149 - protein, 5 aa, Artificial Sequence
  FcepsilonRIgamma hinge region
LGEPQ

SEQ ID NO: 150 - protein, 127 aa, Artificial Sequence
  BTLA hinge region
KESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYR

SEQ ID NO: 151 - protein, 137 aa, Artificial Sequence
  GITR hinge region
QRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEP

SEQ ID NO: 152 - protein, 30 aa, Artificial Sequence
  DAP10 hinge region
QTTPGERSSLPAFYPGTSGSCSGCGSLSLP

SEQ ID NO: 153 - protein, 275 aa, Artificial Sequence
  TIM1 hinge region
SVKVGGEAGPSVTLPCHYSGAVTSMCWNRGSCSLFTCQNGIVWTNGTHVTYRKDTRYKLLGDLSRRDVSLTIENTAVSDSGVYCCRVEHRGWFNDMKITVSLEIVPPKVTTTPIVTTVPTVTTVRTSTTVPTTTTVPMTTVPTTTVPTTMSIPTTTTVLTTMTVSTTTSVPTTTSIPTTTSVPVTTTVSTFVPPMPLPRQNHEPVATSPSSPQPAETHPTTLQGAIRREPTSSPLYSYTTDGNDTVTESSDGLWNNNQTQLFLEHSLLTANTTKG

SEQ ID NO: 154 - protein, 217 aa, Artificial Sequence
  SLAM hinge region
ASYGTGGRMMNCPKILRQLGSKVLLPLTYERINKSMNKSIHIVVTMAKSLENSVENKIVSLDPSEAGPPRYLGDRYKFYLENLTLGIRESRKEDEGWYLMTLEKNVSVQRFCLQLRLYEQVSTPEIKVLNKTQENGTCTLILGCTVEKGDHVAYSWSEKAGTHPLNPANSSHLLSLTLGPQHADNIYICTVSNPISNNSQTFSPWPGCRTDPSETKP

SEQ ID NO: 155 - protein, 367 aa, Artificial Sequence
  CD30 hinge region
FPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAG

SEQ ID NO: 156 - protein, 37 aa, Artificial Sequence
  LIGHT hinge region
MEESVVRPSVFVVDGQTDIPFTRLGRSHRRQSCSVAR

SEQ ID NO: 157 - RNA, 20 nt, Artificial Sequence
  TRAC sg9
agagucucucagcugguaca

SEQ ID NO: 158 - RNA, 20 nt, Artificial Sequence
  TRAC sg10
ugugcuagacaugaggucua

SEQ ID NO: 159 - RNA, 20 nt, Artificial Sequence
  TRAC sg11
cucucagcugguacacggca

SEQ ID NO: 160 - RNA, 20 nt, Artificial Sequence
  TRAC sg12
acacggcagggucaggguuc

SEQ ID NO: 161 - RNA, 20 nt, Artificial Sequence
  TRAC sg13
agcugguacacggcaggguc

SEQ ID NO: 162 - RNA, 20 nt, Artificial Sequence
  TRAC sg14
gagaaucaaaaucggugaau

SEQ ID NO: 163 - RNA, 20 nt, Artificial Sequence
  TRAC sg15
gacaccuucuuccccagccc

SEQ ID NO: 164 - RNA, 20 nt, Artificial Sequence
  TRAC sg16
ucucucagcugguacacggc

SEQ ID NO: 165 - RNA, 20 nt, Artificial Sequence
  TRAC sg17
gcugguacacggcaggguca

SEQ ID NO: 166 - RNA, 20 nt, Artificial Sequence
  TRAC sg18
aaagucagauuuguugcucc

SEQ ID NO: 167 - RNA, 20 nt, Artificial Sequence
  TRAC sg19
cuggggaagaaggugucuuc

SEQ ID NO: 168 - RNA, 20 nt, Artificial Sequence
  TRAC sg20
uggauuuagagucucucagc

SEQ ID NO: 169 - RNA, 20 nt, Artificial Sequence
  TRAC sg21
agagcaacagugcuguggcc

SEQ ID NO: 170 - RNA, 20 nt, Artificial Sequence
  TRAC sg22
cuucaagagcaacagugcug

SEQ ID NO: 171 - RNA, 20 nt, Artificial Sequence
  TRAC sg23
auuuguuugagaaucaaaau

SEQ ID NO: 172 - RNA, 20 nt, Artificial Sequence
  HLA-A02 sg10
cccucguccugcuacucucg

SEQ ID NO: 173 - RNA, 20 nt, Artificial Sequence
  HLA-A02 sg2
cguacuggugguacccgcgg

SEQ ID NO: 174 - RNA, 20 nt, Artificial Sequence
  HLA-A02 sg5
cugaccaugaagccacccug

SEQ ID NO: 175 - RNA, 20 nt, Artificial Sequence
  HLA-A02 sg14
agacucaccgaguggaccug

SEQ ID NO: 176 - RNA, 20 nt, Artificial Sequence
  HLA-A02 sg21
ggacccuccugcucuaucca

SEQ ID NO: 177 - RNA, 20 nt, Artificial Sequence
  HLA-A02 sg23
gauguaauccuugccgucgu

SEQ ID NO: 178 - RNA, 20 nt, Artificial Sequence
  HLA-A02 sg26
ccugcgcucuuggaccgcgg

SEQ ID NO: 179 - RNA, 20 nt, Artificial Sequence
  HLA-A02 sg29
ccucguccugcuacucucgg

SEQ ID NO: 180 - RNA, 20 nt, Artificial Sequence
  HLA-A02 sg30
aacccucguccugcuacucu

SEQ ID NO: 181 - RNA, 20 nt, Artificial Sequence
  HLA-A02 sg31
gaggguucggggcgccauga

SEQ ID NO: 182 - RNA, 20 nt, Artificial Sequence
  HLA-A02 sg32
ccugcuacucucgggggcuc

SEQ ID NO: 183 - RNA, 20 nt, Artificial Sequence
  HLA-A02 sg33
cugguuguaguagccgcgca

SEQ ID NO: 184 - RNA, 20 nt, Artificial Sequence
  HLA-A02 sg34
uagcccacugcgaugaagcg

SEQ ID NO: 185 - RNA, 20 nt, Artificial Sequence
  HLA-A02 sg35
guggaccuggggacccugcg

SEQ ID NO: 186 - RNA, 20 nt, Artificial Sequence
  HLA-A02 sg36
uggacgacacgcaguucgug

SEQ ID NO: 187 - RNA, 20 nt, Artificial Sequence
  HLA-A02 sg37
acagacucaccgaguggacc

SEQ ID NO: 188 - RNA, 20 nt, Artificial Sequence
  HLA-A02 sg38
gcaggaggguccggaguauu

SEQ ID NO: 189 - RNA, 20 nt, Artificial Sequence
  HLA-A02 sg39
aguauugggacggggagaca

SEQ ID NO: 190 - RNA, 20 nt, Artificial Sequence
  HLA-A02 sg40
cucagaccaccaagcacaag

SEQ ID NO: 191 - RNA, 20 nt, Artificial Sequence
  HLA-A02 sg41
ccgccgcgguccaagagcgc

SEQ ID NO: 192 - RNA, 20 nt, Artificial Sequence
  HLA-A02 sg42
cucuuggaccgcggcggaca

SEQ ID NO: 193 - RNA, 20 nt, Artificial Sequence
  HLA-A02 sg43
ggauuacaucgcccugaaag

SEQ ID NO: 194 - RNA, 20 nt, Artificial Sequence
  HLA-A11 sg8-3
cccccgagaguagcaggagg

SEQ ID NO: 195 - RNA, 20 nt, Artificial Sequence
  HLA-A11 sg9-3
aguagcaggaggaggguucg

SEQ ID NO: 196 - RNA, 20 nt, Artificial Sequence
  HLA-A11 sg10-3
cccuccuccugcuacucucg

SEQ ID NO: 197 - RNA, 20 nt, Artificial Sequence
  HLA-A11 sg11-3
ccccgagaguagcaggagga

SEQ ID NO: 198 - RNA, 20 nt, Artificial Sequence
  HLA-A11 sg14-3
agacugaccgaguggaccug

SEQ ID NO: 199 - RNA, 20 nt, Artificial Sequence
  HLA-A11 sg16-3
ggggccggaguauugggacc

SEQ ID NO: 200 - RNA, 20 nt, Artificial Sequence
  HLA-A11 sg20-3
ccacucggucagucugugac

SEQ ID NO: 201 - RNA, 20 nt, Artificial Sequence
  HLA-A11 sg21-3
uggauagagcaggaggggcc

SEQ ID NO: 202 - RNA, 20 nt, Artificial Sequence
  HLA-A11 sg28-3
ugccgucguaggcguccugc

SEQ ID NO: 203 - RNA, 20 nt, Artificial Sequence
  HLA-A11 Rsg2
cguccugccgguacccgcgg

SEQ ID NO: 204 - RNA, 20 nt, Artificial Sequence
  HLA-A11 sg21
ggccccuccugcucuaucca

SEQ ID NO: 205 - RNA, 20 nt, Artificial Sequence
  HLA-A24 sg12-2
ccuggcccugacccagaccu

SEQ ID NO: 206 - RNA, 20 nt, Artificial Sequence
  HLA-A24 sg13-2
cccuggcccugacccagacc

SEQ ID NO: 207 - RNA, 20 nt, Artificial Sequence
  HLA-A24 sg14-2
agacugaccgagagaaccug

SEQ ID NO: 208 - RNA, 20 nt, Artificial Sequence
  HLA-A24 sg16-2
ggggccggaguauugggacg

SEQ ID NO: 209 - RNA, 20 nt, Artificial Sequence
  HLA-A24 sg20-2
ucucucggucagucugugag

SEQ ID NO: 210 - RNA, 20 nt, Artificial Sequence
  HLA-A24 sg21-2
ggccccuccugcucuaucca

SEQ ID NO: 211 - RNA, 20 nt, Artificial Sequence
  HLA-A24 sg22-2
aggcguacuggugguacccg

SEQ ID NO: 212 - RNA, 20 nt, Artificial Sequence
  HLA-A24 sg25-2
cugagccgccauguccgccg

SEQ ID NO: 213 - RNA, 21 nt, Artificial Sequence
  HLA-A-homo-551
gcggagcaguugagagccuac

SEQ ID NO: 214 - RNA, 21 nt, Artificial Sequence
  HLA-A-homo-NEG
gcucagaucaccaagcgcaag

SEQ ID NO: 215 - RNA, 21 nt, Artificial Sequence
  TRAC-homo-375
gaaaguggccggguuuaaucu

SEQ ID NO: 216 - RNA, 21 nt, Artificial Sequence
  TRAC-homo-NEG
gaaacagauacgaaccuaaac